(19) United States
(12) Patent Application Publication
(10) Pub. No.: US 2026/0077033 A1
(43) Pub. Date: Mar. 19, 2026

(54) METHODS FOR TREATING OR PREVENTING RESPIRATORY DISEASES IN CATTLE USING POLYMERS AND POLYPLEXES FOR mRNA DELIVERY AND mRNA THERAPY

(71) Applicants: MISSISSIPPI STATE UNIVERSITY OFFICE OF TECHNOLOGY MANAGEMENT, Starkville, MS (US); GEORGIA TECH RESEARCH CORPORATION, Atlanta, GA (US)

(72) Inventors: Amelia Woolums, Starkville, MS (US); Philip J. Santangelo, Smyrna, GA (US); Daryll Vanover, Atlanta, GA (US)

(73) Assignees: MISSISSIPPI STATE UNIVERSITY OFFICE OF TECHNOLOGY MANAGEMENT, Starkville, MS (US); GEORGIA TECH RESEARCH CORPORATION, Atlanta, GA (US)

(21) Appl. No.: 18/886,686

(22) Filed: Sep. 16, 2024

Related U.S. Application Data
(60) Provisional application No. 63/583,529, filed on Sep. 18, 2023.

(51) Int. Cl. A61K 39/215; A61K 39/00; A61P 37/04

(52) U.S. Cl. CPC A61K 39/215; A61P 37/04; A61K 2039/53; A61K 2039/543; A61K 2039/545; A61K 2039/6093

(57) ABSTRACT

A method of preventing or treating or controlling a respiratory disease in a bovine by delivering to a lung or trachea of the bovine a composition including a polyplex having one or more mRNA encoded with a molecule selected from lycosylphosphatidylinositol (GPI) complex, antibody immunoglobulin G (IgG), antibody immunoglobulin A (IgA), antibody immunoglobulin M (IgM), antimicrobial peptide Bactenecin 5 (Bac5), antimicrobial peptide Bactenecin 7 (Bac7), bovine myeloid antimicrobial peptide (BMAP-28), lipoxin inducing enzymes, resolvin inducing enzymes, cytokines, CRISPR associated protein 13 (Cas13) enzymes, nanoluciferase (NLuc) proteins, and gene activator, catalytically dead CRISPR-associated protein 9 fused to an activator selected from the group consisting of VP64-p65-Rta transactivation domain (dCas9-VPR), VP64, and VP64-p65-HSF1 on the N terminus and SS18 on the C-terminus, and a polymer prepared by polymerizing a diacrylate monomer; one or more linker monomers; and one or more branching monomers; and end-capping the polymer.

### CROSS-REFERENCE OT RELATED APPLICATIONS

This application claims the benefit of U.S. provisional application No. 63/583,529 filed on Sep. 18, 2023, the entire disclosure of which is hereby incorporated by reference in its entirety as if fully set forth herein.

### STATEMENT OF GOVERNMENT INTEREST

This invention was made with government support under Contract Number HR00111920008 awarded by the Department of Defense and the Defense Advanced Research Projects Agency. The Government has certain rights in the invention.

### FIELD OF THE INVENTION

The present invention relates to methods of preventing or treating or controlling a respiratory disease in a bovine by directly delivering a pharmaceutical composition including a messenger RNA (mRNA) and polymers to the lung and methods for making them.

### BACKGROUND OF THE INVENTION

Bovine respiratory disease (BRD) is the leading cause of sickness and death in many types of cattle in the U.S. and around the world. It is caused by a variety of viruses and/or bacteria. Vaccines and antimicrobials are commonly used to prevent and control BRD, and many antimicrobials have been licensed to treat the bacteria that contribute to BRD. Despite these actions, BRD persists as a costly health problem that negatively impacts the well-being and productivity of cattle. An immunostimulant (Zelnate™, marketed by Bayer, which was then purchased by Elanco) has recently been marketed to control BRD but is no longer widely available.

To date, administration of mRNA to the lungs has largely consisted of academic demonstrations of polyethylenimine (PEI) and lipid nanoparticle (LNP) delivery to mice, with few formulations reaching patients.¹⁻⁶ The most prominent clinical trial to date consisted of a nebulized cystic fibrosis transmembrane conductance regulator (CFTR) mRNA (MRT5005™) from Translate Bio, formulated with an LNP, but it failed to produce sufficient protein to improve lung function in CF patients.⁷

Poly-beta-amino-esters (PBAEs) have demonstrated protein expression via nebulizer administration to the lungs of mice with minimal observed toxicity.^{8.9} While the prior PBAE study only focused on expression of reporter constructs, the present invention demonstrates the use of a hyperbranched PBAE for nebulized delivery of mRNA-expressed clustered regularly interspaced short palindromic repeat (CRISPR) associated protein 13 (Cas13a) along with a guide RNA (gRNA).¹⁰ We observed therapeutic levels of Cas13a expression and gRNA delivery in both mice and hamsters against influenza and SARS-COV-2, respectively.

Messenger RNA has been used to vaccinate millions of people. However, the diversity of pulmonary pathologies including infections, genetic disorders, asthma, and others, reveals the lung as an important organ to directly target for future mRNA therapeutics and preventatives.

### SUMMARY OF THE INVENTION

In one aspect, the present invention relates to preventing a respiratory disease in a bovine comprising delivering polymeric nanoparticle formulations containing mRNA for functional delivery to the lungs or trachea of a bovine.

In another aspect, the present invention relates to treating a respiratory disease in a bovine comprising delivering polymeric nanoparticle formulations containing mRNA for functional delivery to the lungs or trachea of a bovine.

In another aspect, the present invention relates to controlling a respiratory disease in a bovine comprising delivering polymeric nanoparticle formulations containing mRNA for functional delivery to the lungs or trachea of a bovine. The polymeric nanoparticle formulations are obtained using combinatorial synthesis. The formulations may be administered, for example, using a low dead-volume, nose-only inhalation system originally developed for mice.

The polymeric nanoparticles may include a polymer according to P76, a poly-beta-amino-thio-ester polymer, to provide increased expression in comparison to similar formulations lacking the thiol.

The methods of the invention are applicable for delivery to different animal species with varying RNA cargos, and low toxicity. For example, P76 allows for dose sparing when delivering a mRNA-expressed Cas13a-mediated treatment in a SARS-COV-2 challenge model, resulting in similar efficacy to a 20-fold higher dose of a neutralizing antibody. In addition, the combinatorial synthesis approach of the invention should be useful for the discovery of other promising polymeric formulations for future RNA pharmaceutical development for direct treatment of the lung.

The present invention demonstrates that messenger RNA (mRNA) can be applied directly to the respiratory tract through the nasal passages of animals, such as bovine, to cause the production of molecules, such as antimicrobial peptides or antibody molecules, on the respiratory surface. These molecules can prevent or counteract the effects of specific or nonspecific infection by viruses and bacteria that contribute to BRD, wherein specific provides protection for a single virus or bacteria and nonspecific provides protection to multiple viruses or bacteria. Importantly, mRNA therapy can induce production of antibodies within hours of treatment, which is significantly faster than results obtained by vaccination. In addition, the application of the mRNA in combination with the polymer of the present invention may be used to induce the production of the antimicrobial peptides or antibody molecules to speed up the resolution of an existing respiratory disease.

Moreover, mRNA-induced production of antimicrobial peptides can be used to treat bacterial BRD without increasing resistance to antimicrobial drugs important for animals and humans. This is important because antimicrobial resistance is a negative consequence of the current antimicrobial approaches used to control BRD.

In summary, mRNA treatment of the bovine respiratory tract has the potential to induce protective responses to BRD within hours and without increasing antimicrobial resistance, thereby providing a way to control BRD that is superior to currently used approaches.

The present method is most comparable to the administration of vaccines to induce specific immunity, or immunostimulants to induce nonspecific immunity. However, vaccines take several days to be effective, and immunostimulants, as used in animals, are administered into the muscle or under the skin, not directly into the respiratory tract.

Thus, the present invention is more rapid and more targeted/focused than competing approaches to prevent or treat respiratory disease.

The following sentences may be used to describe the invention:
1. In a first aspect, the present invention relates to a method of preventing or treating or controlling a respiratory disease in a bovine comprising:
delivering to a lung or trachea of the bovine a pharmaceutical composition comprising a pharmaceutically acceptable carrier and a polyplex,
   wherein the polyplex comprises:
   one or more, optionally further modified, mRNA encoded with a molecule selected from the group consisting of glycosylphosphatidylinositol (GPI) complex, antibody immunoglobulin G (IgG), antibody immunoglobulin A (IgA), antibody immunoglobulin M (IgM), antimicrobial peptide Bactenecin 5 (Bac5), antimicrobial peptide Bactenecin 7 (Bac7), bovine myeloid antimicrobial peptide (BMAP-28), lipoxin inducing enzymes, resolvin inducing enzymes, cytokines, CRISPR associated protein 13 (Cas13) enzymes, nanoluciferase (NLuc) proteins, and gene activator, catalytically dead CRISPR-associated protein 9 fused to an activator selected from the group consisting of VP64-p65-Rta transactivation domain (dCas9-VPR), VP64, and VP64-p65-HSF1 on the N terminus and SS18 on the C-terminus,
   a polymer prepared by
   a. polymerizing components i-ii and optionally iii:
      i. a diacrylate monomer;
      ii. one or more linker monomers including one or more functional groups independently selected from the group consisting of amino groups, alcohol groups and thiol groups; and
      iii. one or more branching monomers including one or more functional groups independently selected from the group consisting of amino groups, alcohol groups and thiol groups; and
   b. end-capping the product of step (a) with one or more end capping monomers including one or more functional groups independently selected from the group consisting of amino groups and alcohol groups.
2. The method of sentence 1, wherein the pharmaceutical composition may further comprise an RNA, an antibody, a cytokine, or combinations thereof;
wherein the RNA is selected from the group consisting of messenger RNA (mRNA), cargo RNA (cRNA), guide RNA (gRNA), transfer RNA (tRNA), ribosomal RNA (rRNA), GPI anchored RNA, and CRISPR RNA (crRNA), optionally, the RNA is selected from the group consisting of mRNA, CRNA, gRNA, anchored RNA, and crRNA;
wherein the antibody is selected from the group consisting of IgG, IgA, IgM; and
wherein the cytokine is optionally selected from the group consisting of interferon and lambda.
3. The method of any one of claims 1-2, wherein the pharmaceutical composition may comprise two or more mRNA wherein each mRNA is individually encoded with a molecule selected from the group consisting of glycosylphosphatidylinositol (GPI) complex, antibody immunoglobulin G (IgG), antibody immunoglobulin A (IgA), antibody immunoglobulin M (IgM), antimicrobial peptide Bactenecin 5 (Bac5), antimicrobial peptide Bactenecin 7 (Bac7), bovine myeloid antimicrobial peptide (BMAP-28), lipoxin inducing enzymes, resolvin inducing enzymes, cytokines, CRISPR associated protein 13 (Cas13) enzymes, nanoluciferase (NLuc) proteins, and gene activator, catalytically dead CRISPR-associated protein 9 fused to an activator selected from the group consisting of VP64-p65-Rta transactivation domain (dCas9-VPR), VP64, and VP64-p65-HSF1 on the N terminus and SS18 on the C-terminus, or optionally, wherein the molecule is selected from the group consisting of GPI, Cas13, NLuc, and dCas9-VPR.
4. The method of any one of sentences 1-3, wherein the pharmaceutical composition may comprise three or more mRNA wherein each mRNA is individually encoded with a molecule selected from the group consisting of glycosylphosphatidylinositol (GPI) complex, antibody immunoglobulin G (IgG), antibody immunoglobulin A (IgA), antibody immunoglobulin M (IgM), antimicrobial peptide Bactenecin 5 (Bac5), antimicrobial peptide Bactenecin 7 (Bac7), bovine myeloid antimicrobial peptide (BMAP-28), lipoxin inducing enzymes, resolvin inducing enzymes, cytokines, CRISPR associated protein 13 (Cas13) enzymes, nanoluciferase (NLuc) proteins, and gene activator, catalytically dead CRISPR-associated protein 9 fused to an activator selected from the group consisting of VP64-p65-Rta transactivation domain (dCas9-VPR), VP64, and VP64-p65-HSF1 on the N terminus and SS18 on the C-terminus; or wherein the molecule is selected from the group consisting of GPI, Cas 13, NLuc, and dCas9-VPR.
5. The method of any one of sentences 1-4, wherein the pharmaceutical composition may further comprise crRNA.
6. The method of any one of sentences 1-5, wherein the pharmaceutical composition may be delivered via a nebulizer, vibrating mesh nebulizer, jet nebulizer, intratracheal instillation, intranasal instillation, and aerosol or instillation to oral, nasal, and pharyngeal membranes.
7. The method of sentence 6, wherein the pharmaceutical composition may be delivered via a nebulizer which produces nebulized droplets have a diameter of from about 1 μm-10 μm, as measured by dynamic light scattering.
8. The method of any one of sentences 1-7, wherein the bovine may be selected from the group consisting of calf, heifer, adult cow, steer, and bull
9. The method of any one of sentences 1-8, wherein the respiratory disease may be selected from the group consisting of pneumonia, pleuritis, pleuropneumonia, bronchitis, respiratory syncytial virus (RSV) infection, influenza, coronavirus, infectious bovine herpes, bovine viral diarrhea virus, influenza, bovine rhinovirus, bovine rhinitis virus, Mannheimia haemolytica, Pasteurella multocida, Histophilus somni, Mycoplasma species, etc.
10. The method of any one of sentences 1-9, wherein the method may comprise treating or controlling the respiratory disease wherein the bovine is already infected with the respiratory disease.
11. The method of any one of sentences 1-10, wherein the polymer may comprise the one or more branching monomers.
12. The method of sentence 11, wherein the polymer may have a molar ratio of the diacrylate monomer to the one or more linker monomers to the one or more branching monomers to the one or more end capping monomers is from about 1.0:0.25:0:1.0 to 1.0:1.0:1.0:2.0, or from about 1.0:0.4:0.1:0.5 to 1.0:0.75:0.75:1.5.
13. The method of any one of sentences 1-12, wherein the one or more diacrylates of the polymer may be selected from the group consisting of:

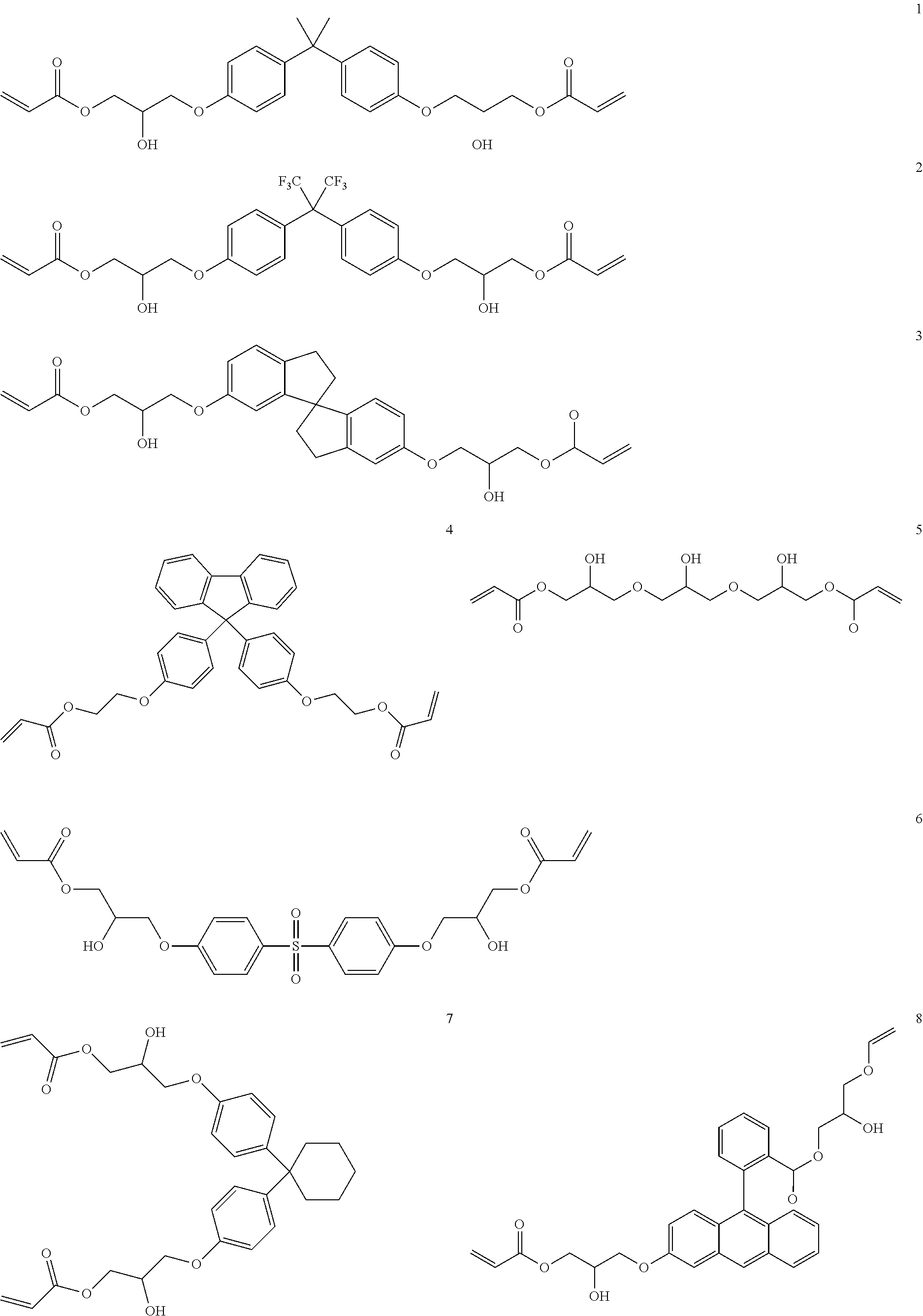

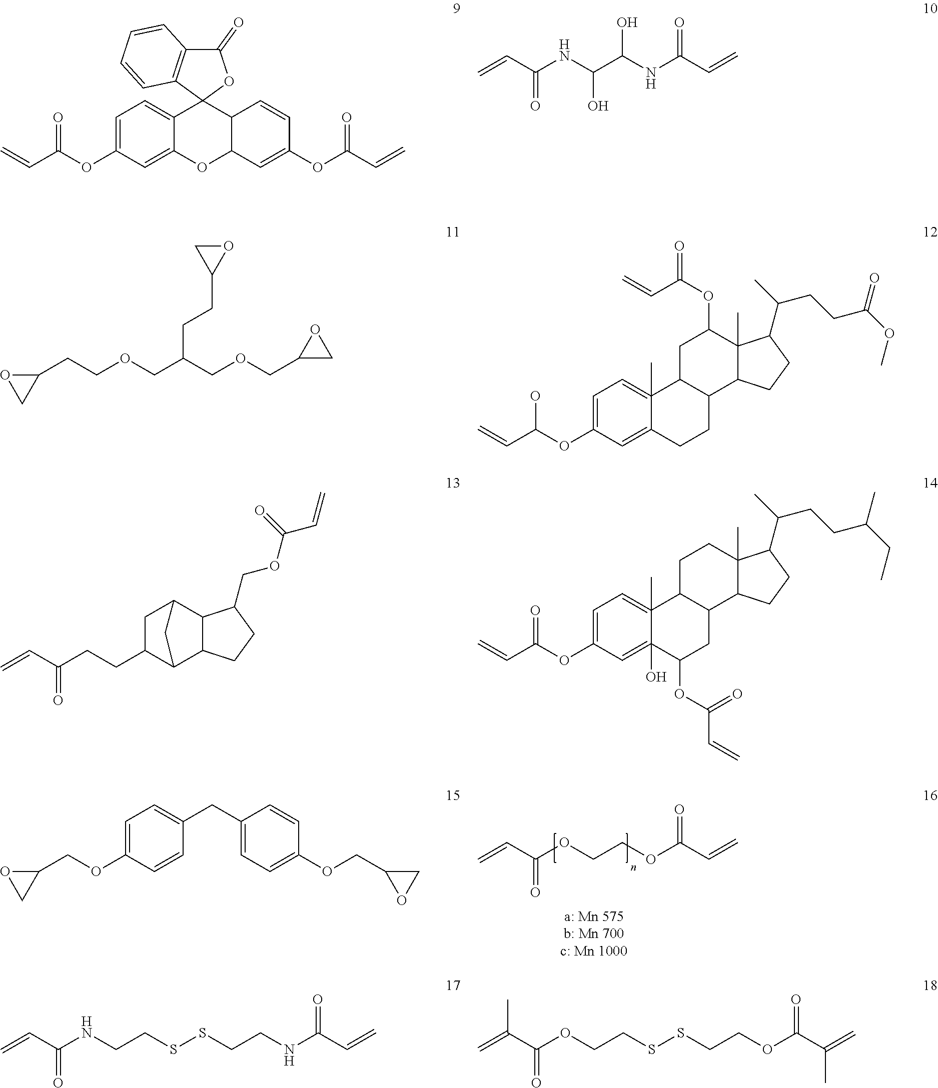

14. The method of any one of sentences 1-13, wherein the one or more linker monomers may be selected from the group consisting of:

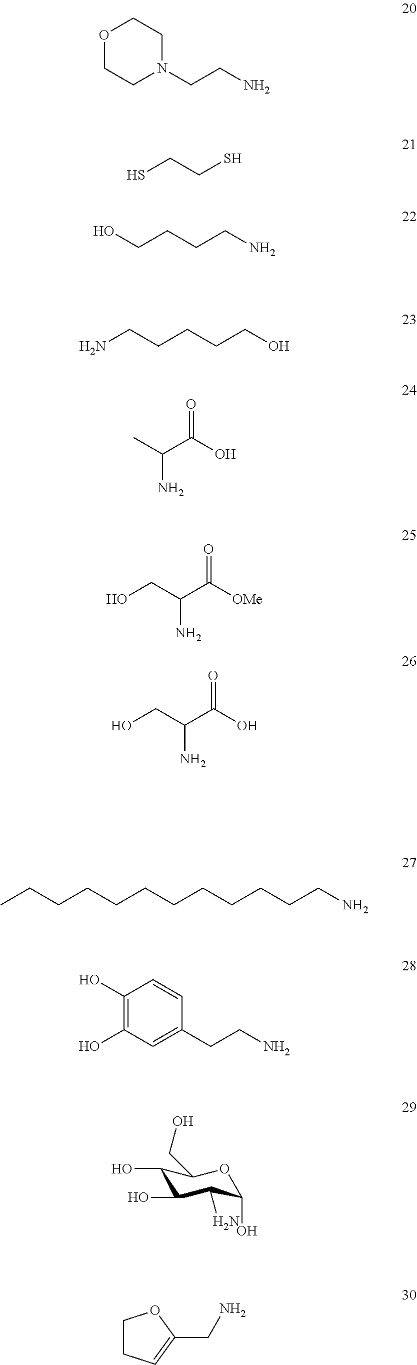

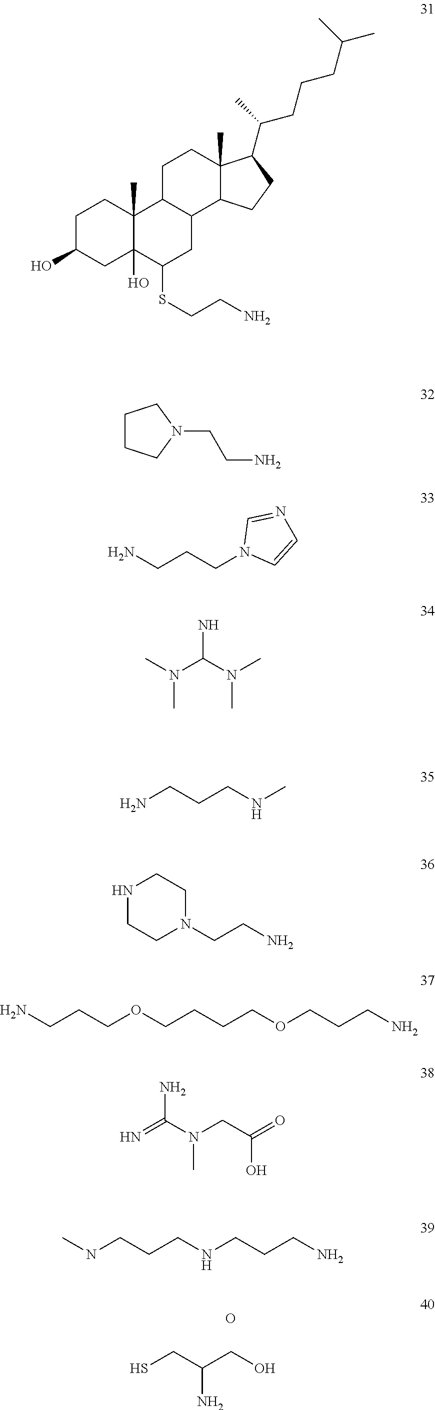

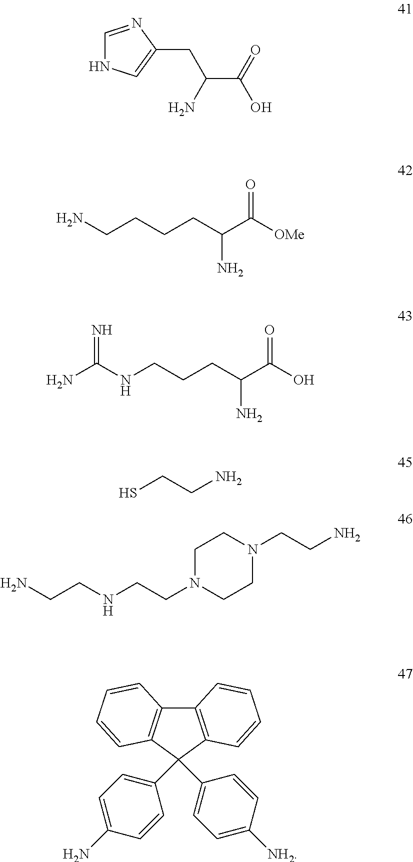

15. The method of any one of sentences 1-14, wherein the one or more branching monomers may be selected from the group consisting of:

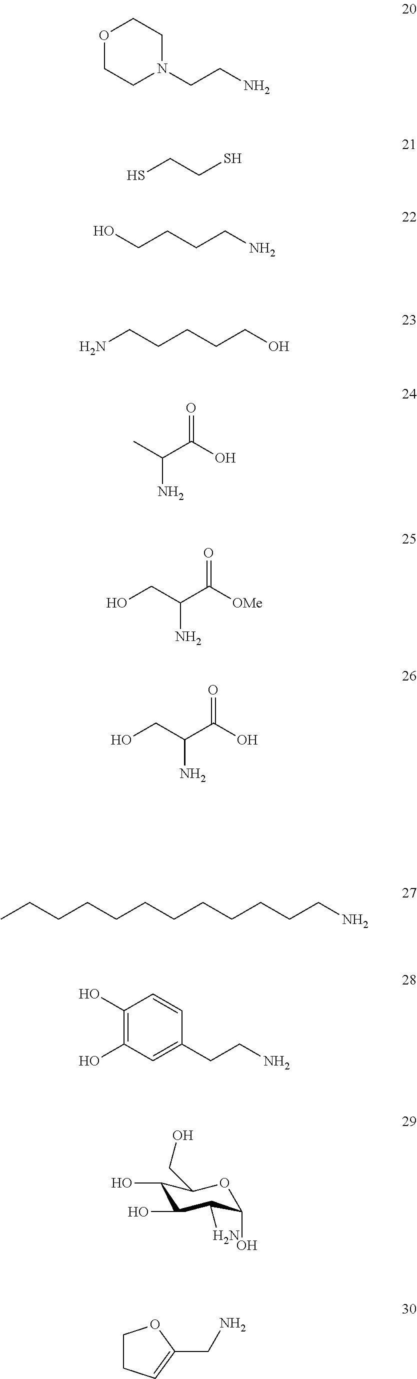

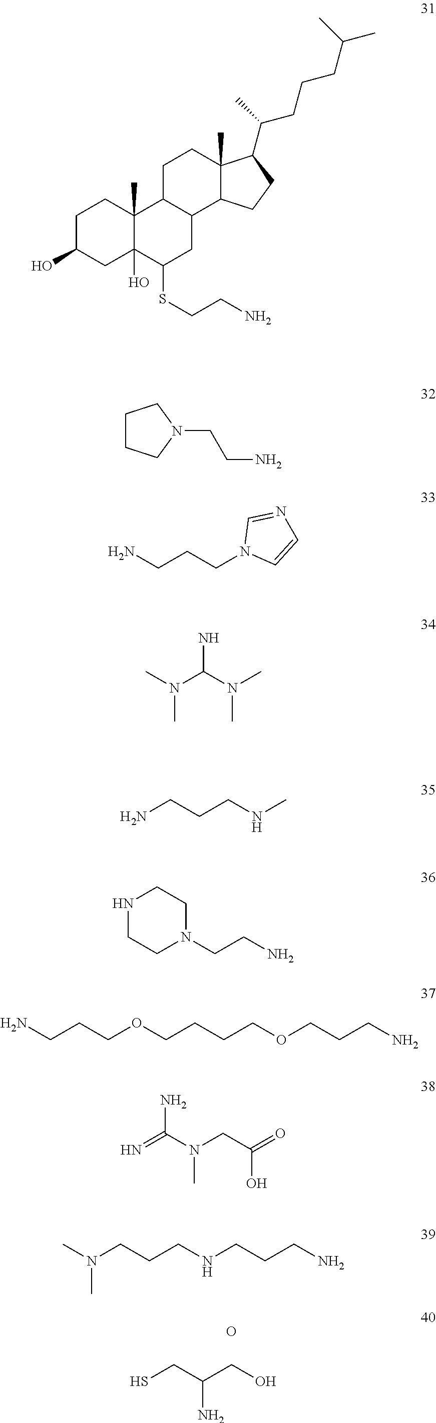

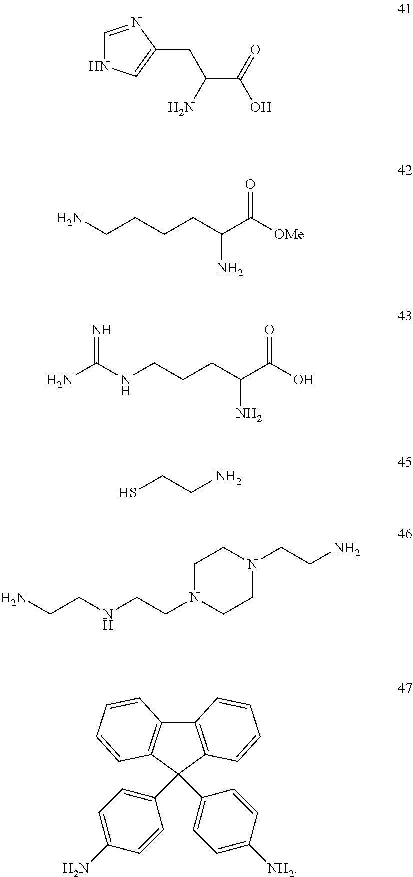

16. The method of any one of sentences 1-15, wherein the one or more end capping monomers may be selected from the group consisting of:

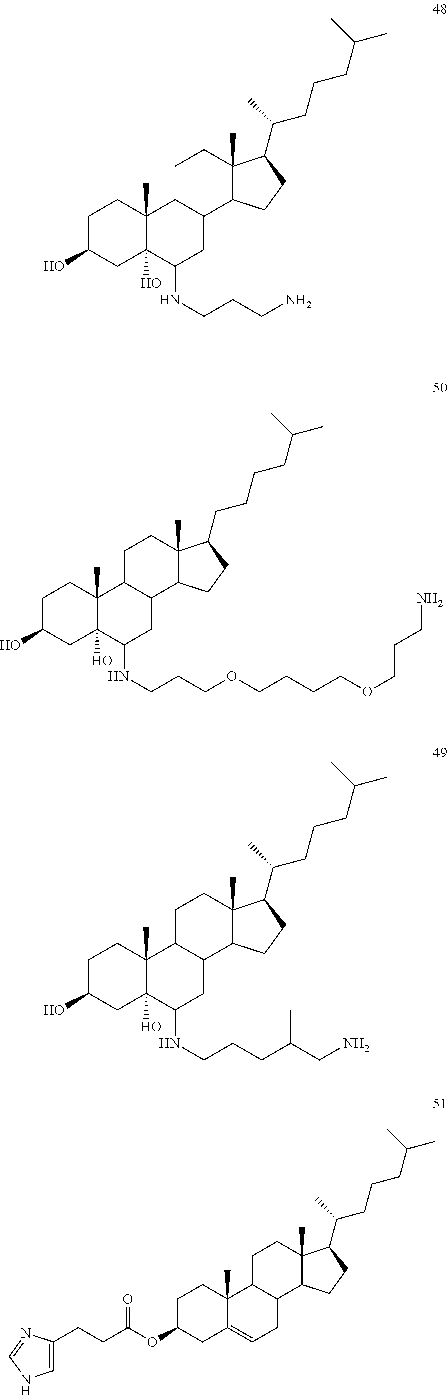

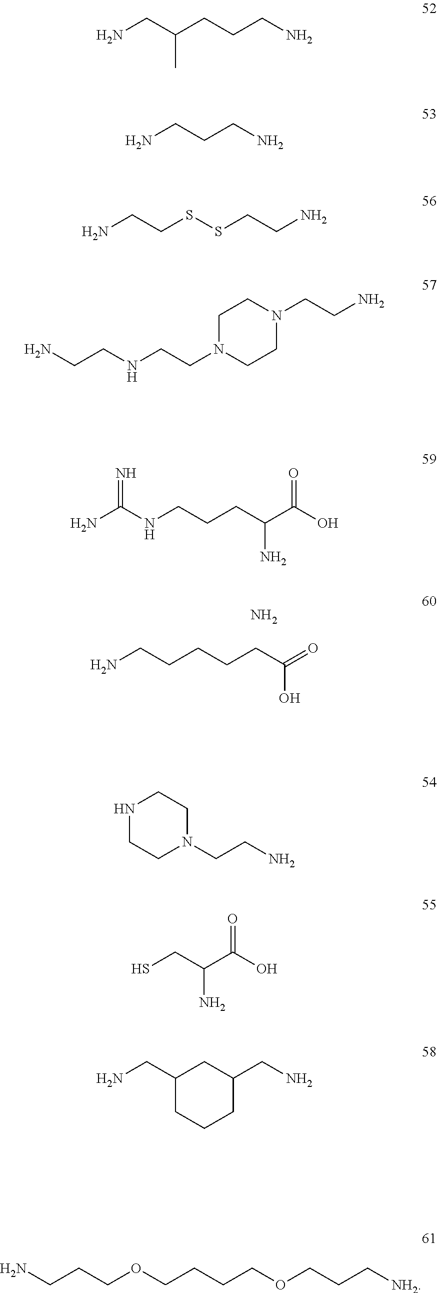

17. The method of any one of sentences 1-16, wherein the polymer may be selected from the group consisting of:

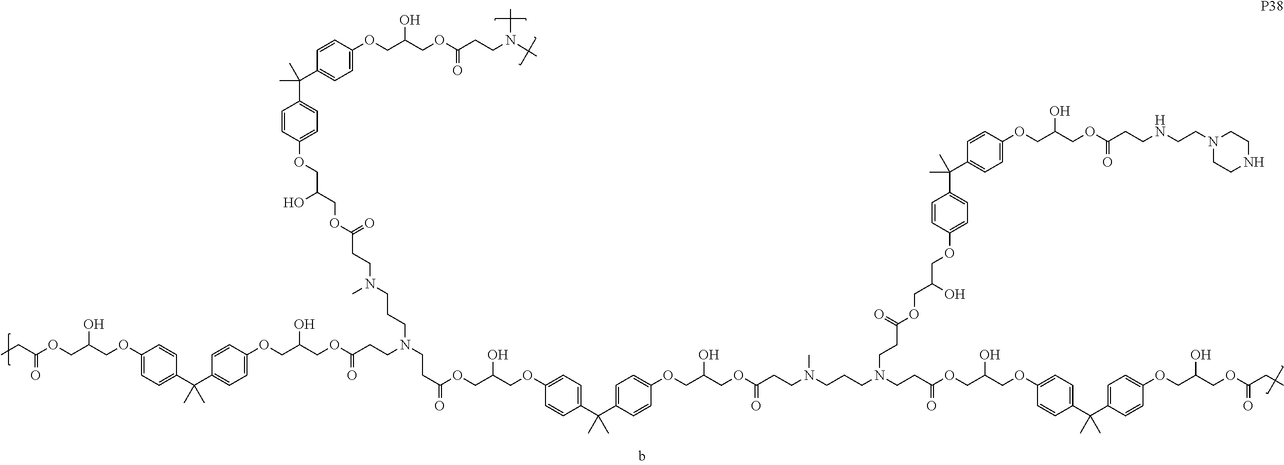

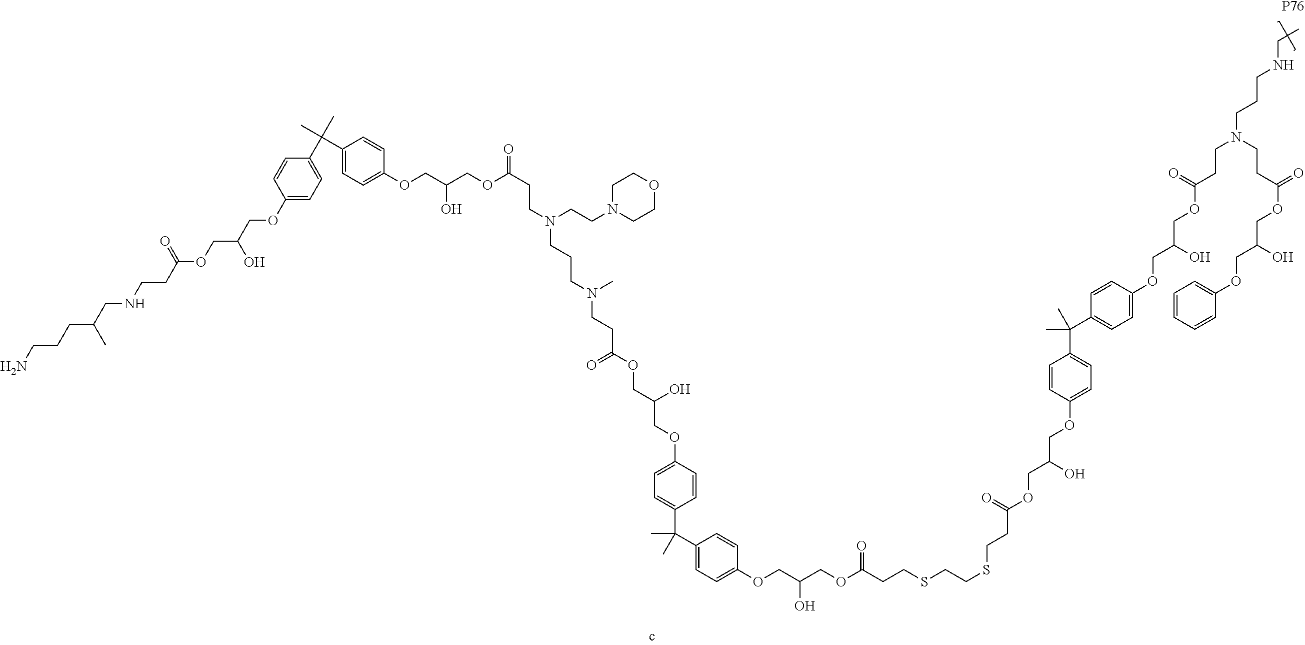 [SMILES: CCNCCCN(CCC(=O)OCC(O)COc1ccccc1)CCC(=O)OCC(O)COc1ccc(C(C)(C)c2ccc(OCC(O)COC(=O)CCSCCSCCC(=O)OCC(O)COc3ccc(C(C)(C)c4ccc(OCC(O)COC(=O)CCN(C)CCCN(CCC(=O)OCC(O)COc5ccc(C(C)(C)c6ccc(OCC(O)COC(=O)CCNCC(C)CCCN)cc6)cc5)CCN5CCOCC5)cc4)cc3)cc2)cc1 ; "P76"]

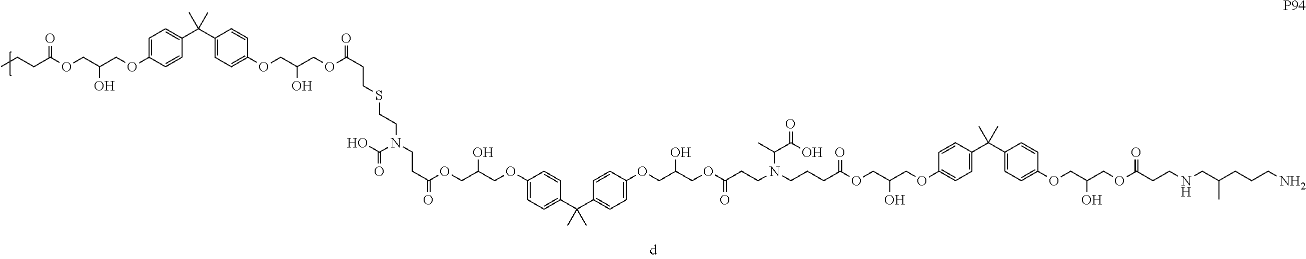 [SMILES: CCCC(=O)OCC(O)COc1ccc(C(C)(C)c2ccc(OCC(O)COC(=O)CCSCCN(CCC(=O)OCC(O)COc3ccc(C(C)(C)c4ccc(OCC(O)COC(=O)CCN(CCCC(=O)OCC(O)COc5ccc(C(C)(C)c6ccc(OCC(O)COC(=O)CCNCC(C)CCCN)cc6)cc5)C(C)C(=O)O)cc4)cc3)C(=O)O)cc2)cc1 ; "P94"]

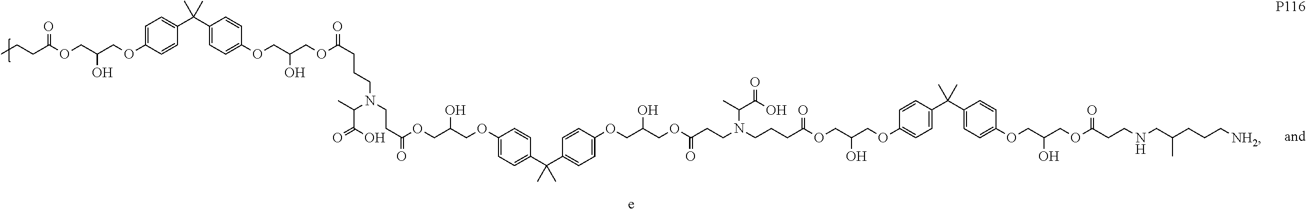 [SMILES: CCCC(=O)OCC(O)COc1ccc(C(C)(C)c2ccc(OCC(O)COC(=O)CCCN(CCC(=O)OCC(O)COc3ccc(C(C)(C)c4ccc(OCC(O)COC(=O)CCN(CCCC(=O)OCC(O)COc5ccc(C(C)(C)c6ccc(OCC(O)COC(=O)CCNCC(C)CCCN)cc6)cc5)C(C)C(=O)O)cc4)cc3)C(C)C(=O)O)cc2)cc1 ; "P116"]

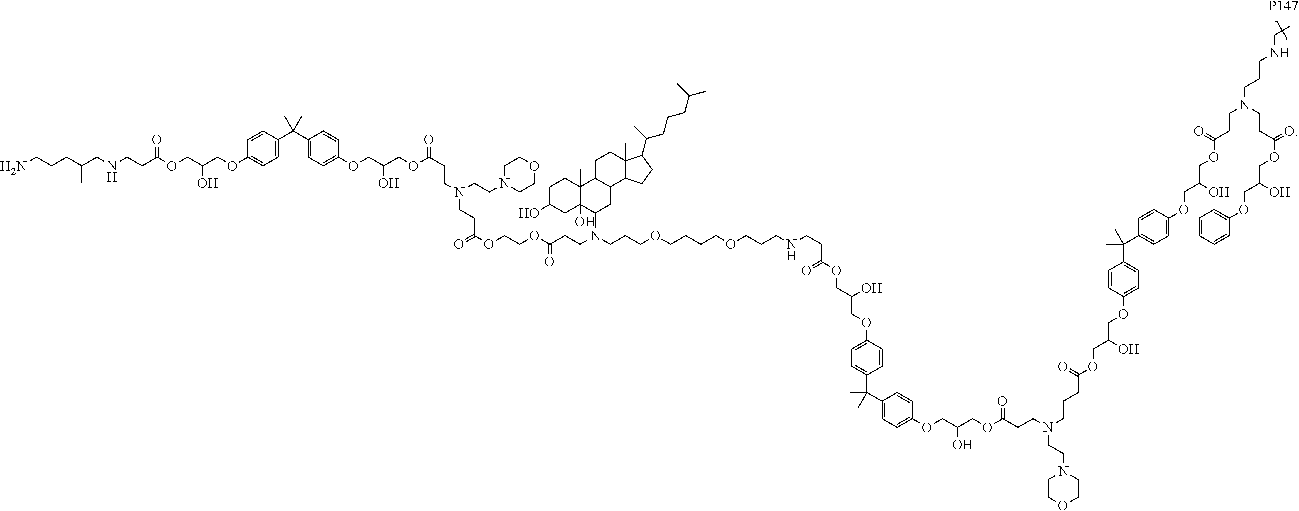 [SMILES: CCNCCCN(CCC(=O)OCC(O)COc1ccccc1)CCC(=O)OCC(O)COc1ccc(C(C)(C)c2ccc(OCC(O)COC(=O)CCCN(CCC(=O)OCC(O)COc3ccc(C(C)(C)c4ccc(OCC(O)COC(=O)CCNCCCOCCCCOCCCN(CCC(=O)OCCOC(=O)CCN(CCC(=O)OCC(O)COc5ccc(C(C)(C)c6ccc(OCC(O)COC(=O)CCNCC(C)CCCN)cc6)cc5)CCN5CCOCC5)C5CC6C7CCC(C(C)CCCC(C)C)C7(C)CCC6C6(C)CCC(O)CC56O)cc4)cc3)CCN3CCOCC3)cc2)cc1 ; "P147"]

18. The method of any one of sentences 1-17, wherein the polymer may be selected from the group consisting of a poly-beta-amino-thio-ester-polymer and a poly-beta-amino-ester polymer.
19. The method of any one of sentences 1-18, wherein the one or more mRNA may be encoded with a molecule selected from the group consisting of glycosylphosphatidylinositol (GPI) complex, antibody immunoglobulin G (IgG), antibody immunoglobulin A (IgA), antibody immunoglobulin M (IgM), antimicrobial peptide Bactenecin 5 (Bac5), antimicrobial peptide Bactenecin 7 (Bac7), bovine myeloid antimicrobial peptide (BMAP-28), lipoxin inducing enzymes, resolvin inducing enzymes, cytokines, CRISPR associated protein 13 (Cas13) enzymes, nanoluciferase (NLuc) proteins, and gene activator, catalytically dead CRISPR-associated protein 9 fused to an activator selected from the group consisting of VP64-p65-Rta transactivation domain (dCas9-VPR), VP64, and VP64-p65-HSF1 on the N terminus and SS18 on the C-terminus; or optionally, the one or more mRNA is encoded with Cas13 enzymes, wherein the Cas13 enzymes may be selected from the group consisting of Cas13a, LbuCas13a, Cas13b, and Cas13d.
20. The method of any one of sentences 1-19, wherein the mRNA may further comprise one or more guide RNAs for an RNA-guided nuclease.
21. The method of any one of sentences 1-20, wherein the one or more mRNA may comprise the mRNA-expressed clustered regularly interspaced short palindromic repeat associated protein 13 (Cas13a).
22. The method of any one of sentences 1-21, wherein the one or more mRNA, or two or more mRNA, or three or more mRNA may be modified to include anchoring sequences to form an anchored mRNA or to include tethering sequences to form a tethered mRNA.
23. The method of sentence 22, wherein the one or more mRNA molecules may be encoded with an anchored nanoluciferase (aNLuc).
24. The method of any one of sentences 1-12, wherein the polyplex has a diameter of from about 50 nm to about 500 nm, or from about 75 nm to about 400 nm, or from about 100 nm to about 200 nm, as measured by Dynamic Light Scattering.
25. The method of any one of sentences 1-24, wherein the pharmaceutical composition excludes the presence of polymer 76.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1G show functional screening of polymers for nebulized mRNA delivery.

FIG. 1A shows polymers synthesized using one or more backbone components, one or more linker components, one or more branching components, and one or more end-capping structures.

FIG. 1B shows one or more cargo RNAs formulated into polyplexes of approximately 100 nm-200 nm in diameter. This colloidal mixture was nebulized, creating droplets having a diameter of ˜4-6 mm, and inhaled.

FIG. 1C shows the polymer screening setup. The polyplexes were nebulized into mice, and the lungs were isolated and analyzed by luminescence.

FIGS. 1D and 1E show bioluminescence results obtained using nose-only nebulization of mRNA-encoded anchored nanoluciferase (aNLuc). FIG. 1D shows images of the bioluminescence and FIG. 1E shows fold change total flux of lungs 24 hours after delivery in mice using hDD90-118 PBAE at the indicated doses. **p<0.01 (one-way ANOVA with multiple comparisons on log-transformed data), where n=3. The bars represent geometric mean. p-values are shown in Table 3.

FIGS. 1F and 1G show representative polymer formulation screening results. FIG. 1F shows images of the bioluminescence and FIG. 1G shows fold change total flux of lungs 24 hours after aNLuc mRNA nebulization into mice using the indicated polymers at 5 mg/kg, where n=2 mice per group. Additional screens are shown in FIGS. 9A-9K and polymer ID definition is found in Table 1. P-values are shown in Table 2.

FIGS. 2A-2D show a comparison of the tested polymers.

FIGS. 2A and 2B show bioluminescence measurements obtained using the polymers.

FIG. 2A shows images of the bioluminescence.

FIG. 2B shows fold change total flux of lungs 24 hours after aNLuc mRNA nebulization into mice using the indicated polymers at 5 mg/kg. ***p<0.001 compared to all other groups (one-way analysis of variance (ANOVA) with multiple comparisons on log-transformed data). Bars indicate geometric mean±SD.

FIG. 2C shows representative microscopy of mouse lungs 4 hours after nebulized delivery using specific polymers. Fluorescence in situ hybridization (FISH) for aNLuc, Scgb1a1 marker of airway cells, and nuclei stained with 4′,6-diamidino-2-phenylindole (DAPI). The scale bar represents 15 mm, where n=2 mice per group. Larger lung areas are presented in FIG. 14.

FIG. 2D shows quantification of mean intensity of aNLuc positive RNA granules in lungs from FIG. 2C. Bars represent means. n=2 mice per group. Percent area measurements are shown in FIG. 15. All p-values are shown in Table 3.

FIGS. 3A-3I show P76 delivered cargo of any size to the lungs of mice with minimal toxicity. P76 delivered a large range of cargo lengths in DBA/2 and BALB/c mice. Lungs were evaluated at 24 hours post transfection using the indicated doses of mRNA or mRNA and clustered regularly interspaced short palindromic repeat RNA (crRNA) formulated with P76.

FIG. 3A shows images of the bioluminescence.

FIG. 3B shows fold change total flux.

FIG. 3C shows weight change of mice transfected with aHCA-NLuc mRNA.

FIG. 3D shows images of the bioluminescence.

FIG. 3E shows fold change total flux.

FIG. 3F shows weight change of mice transfected with Cas 13-NLuc mRNA and crRNA.

FIG. 3G shows images of the bioluminescence.

FIG. 3H shows fold change total flux.

FIG. 3I shows weight change of mice transfected with dCas9-VPR-NLuc mRNA and crRNA. n=3 mice per group. *p<0.05 (two-way ANOVA with multiple comparisons on log-transformed data). For luminescence data, bars indicate geometric mean±SD. For weight data, bars indicate mean±SD. A horizontal line represents no weight change. All p-values are shown in Table 3.

FIGS. 4A-4F show that nebulized P76 delivery was minimally toxic. aNLuc mRNA was delivered via P76 at 1.25 mg/kg. Acetate buffer was delivered to the control group. Mice were sacrificed at days 1, 7, 14, and 21 for terminal blood and tissue collection. For each time point, n=6 mice weights were measured. At indicated days, blood draws were performed on all mice; n=3 mice lungs were used for NanoString analysis and n=3 mice lungs were used for histopathology analysis.

FIG. 4A shows weights as a percentage of starting weight. Error bands represent ±SEM.

FIG. 4B shows a schematic of an enzyme-linked immunoassay (ELISA) to detect mouse anti-P76 polyplex antibody responses at days 1, 7, 14, and 21.

FIG. 4C shows that mouse anti-P76 polyplex antibodies were detected via ELISA. Bars represent the mean. No significant difference was measured by one-way ANOVA (p=0.6450).

FIG. 4D shows complete blood chemistry metrics. *p<0.05, **p<0.01 (one-way ANOVA with Dunnett's multiple comparisons). Gray regions represent 95% confidence interval of naïve mice from Charles River. Samples with insufficient sera or excessive hemolysis were excluded from the analysis. Bars in all graphs indicate mean±standard deviation (SD).

FIG. 4E shows differential gene expression of 561 inflammatory genes measured by NanoString analysis of the indicated time points versus the control. The horizontal line represents p=0.05 (two-tailed t-test on log-transformed normalized data) and vertical lines indicate fold changes of ±2.

FIG. 4F shows representative hematoxylin and eosin (H&E)-stained lung sections from mice at the indicated time points. Scale bar represents 100 mm. n=2 mice per group. All p-values are shown in Table 3.

FIGS. 5A-50 show P76 delivered RNA cargo in a species agnostic manner with minimal toxicity.

FIGS. 5A-5D show that P76 efficiently delivered mRNA to mouse, hamster, and ferret lungs.

FIG. 5A shows images of the bioluminescence.

FIG. 5B shows average radiance.

FIG. 5C shows total area.

FIG. 5D shows total flux of lungs at 24 hours post transfection of aNLuc mRNA at 0.3 mg/kg using P76. n=2 animals per species. Bars indicate geometric mean (average radiance and total flux) or mean (weight).

FIG. 5E shows a luminescence image of a section of a cow's left diaphragmatic lung 24 hours after transfection of aNLuc mRNA at 0.03 mg/kg using P76. Additional images are shown in FIG. 21.

FIGS. 5F and 5G show that nebulized P76 formulations delivered RNA throughout all lung lobes of rhesus macaques.

FIG. 5F shows images of the bioluminescence.

FIG. 5G shows average radiance of lungs at 4 and 24 hours post transfection of aNLuc mRNA at 0.3 mg/kg using P76. The control animal was untreated. n=1 per group.

FIGS. 5H-5K show that P76 delivered RNA cargo in a linear dose-dependent manner with minimal toxicity.

FIG. 5H shows images of the bioluminescence.

FIG. 5I shows average radiance of ferret lungs 24 hours post transfection of aHCA-NLuc mRNA at indicated doses.

FIG. 5J shows images of the bioluminescence.

FIG. 5K shows average radiance of ferret lungs 24 hours post transfection of Cas13-NLuc mRNA at indicated doses. Blue lines represent linear regression, dotted line represents average radiance of control animal. n=2 ferrets per group.

FIGS. 5L and 5M show representative FISH microscopy of ferret lungs in FIG. 5L, and macaque lungs in FIG. 5M, 4 hours post transfection of indicated dose of RNA cargo (white) using P76. Scgb1a1 and Foxj1 airway markers (magenta) and nuclei (DAPI, cyan) for context. Scale bars represent 200 mm, in FIG. 5L and 60 mm, in FIG. 5M. In FIG. 5L, n=2 ferrets per group, and in FIG. 5M, n=1 macaque per group. Full section images of macaque lungs are in FIG. 23.

FIG. 5N shows histology of ferret lungs 24 hours post transfection of 0.3 mg/kg of indicated RNA cargo using P76. n=2 ferrets per group.

FIG. 5O shows histology of macaque lungs 24 hours post transfection of 0.3 mg/kg of aNLuc mRNA using P76. Scale bars represent 100 mm. n=1 macaque per group.

FIGS. 6A-6I show that P76 efficiently delivered mRNA and crRNA and prevented SARS-CoV-2 infection in hamsters.

FIG. 6A shows that nebulized P76 formulations were more efficient than hDD90-118. Quantification of hamster lung luminescence at 24 hours post transfection of 1.25 mg/kg of Cas13a-NLuc mRNA. Bars represent geometric mean+SD. n=2. **p<0.01 (one-way ANOVA with Dunnett's multiple comparisons on log-transformed data).

FIGS. 6B and 6C show nebulized P76 formulations were more efficient than hDD90-118 at delivering RNA cargo of different length. FIG. 6B shows images of the bioluminescence and FIG. 6C shows fold change total flux of hamster lungs 24 h after delivery of indicated total dose of Cas13-NLuc mRNA and crRNA. Bars represent geometric mean+SD. n=2. ***p<0.001 (one-way ANOVA with Šídák's multiple comparisons on log-transformed data)

FIGS. 6D-6I show that P76 delivery of Cas13 mRNA along with N3.2 guide prevented SARS-COV-2 infection in the hamster model. FIG. 6D shows hamsters treated with either nebulized P76 or hDD90-118 formulated Cas13 mRNA and crRNA. 20 hours later, hamsters were inoculated intranasally (IN) with 103 plaque forming units (PFU) of WA-1 SARS-COV-2. Hamsters were euthanized on day 5 and lungs were extracted and processed for viral load quantification. n=8 hamsters per group.

FIG. 6E shows the percent normalized hamster weight over time. Symbols and error bars represent mean % weight±SEM, respectively.

FIG. 6F shows the percent hamster weight at day 5 post infection. Bars represent mean±SD.

FIG. 6G shows the percent normalized hamster weight over time. Symbols and error bars represent mean % weight±SEM, respectively.

FIG. 6H shows the percent hamster weight at day 5 post infection. Bars represent mean±SD.

FIG. 6I shows the percent knockdown of SARS-COV-2 RNA in the lung at day 5 post infection. Bars represent mean±SD. *p<0.05, **p<0.01, ***p<0.001 (one-way ANOVA with Dunnett's multiple comparisons). n=4-5 per group. All p-values are shown in Table 3.

FIGS. 7A-7E show a synthesis diagram and monomers used for polymer synthesis.

FIG. 7A shows a synthesis diagram demonstrating the polymer construction method.

FIG. 7B shows the backbone monomers.

FIG. 7C shows the linker and/or branching monomers.

FIG. 7D shows the end-capping monomers.

FIG. 7E shows linker and/or end-capping monomers.

FIGS. 8A-8B show acrylate ring-opening. FIGS. 8A-8B show a synthesis fragment where

FIG. 8A is a structure of an acrylate ring-opened from a corresponding epoxide and the structure according to FIG. 8B contains approximately 11% of the regioisomer.

FIGS. 9A, 9B, 9C, 9D, 9E, 9F, 9G, 9H, 9I, 9J and 9K show additional screens performed using the polymers and a nose cone nebulizer apparatus. All of these screens included untreated animals and hDD90-118 treated animals as negative and positive controls, respectively. Bars indicate mean±SD. n=2 mice per group.

FIG. 10 shows exemplary monomers and molar equivalent ratios used in the synthesis of polymers.

FIGS. 11A-11E show theoretical structures of polymers.

FIG. 11A shows the theoretical structure of polymer P38.

FIG. 11B shows the theoretical structure of polymer P76.

FIG. 11C shows the theoretical structure of polymer P94.

FIG. 11D shows the theoretical structure of polymer P116.

FIG. 11E shows the theoretical structure of polymer P147.

FIGS. 12A, 12B, 12C, 12D and 12E show nuclear magnetic resonance (NMR) spectra of polymers P38, P76, P94, P116, and P147, respectively.

FIGS. 13A-13B show NMR analysis which revealed N-formyl substitution using dimethylformamide (DMF) solvent for polymer synthesis.

FIG. 13A shows ¹H NMR of the polymers, P38, P76, P94, P116, and P147, in the region characteristic of N-formyl (RR′N—CHO) substitution.

FIG. 13B shows ¹³C NMR of hDD90-118 prepared in the presence of 10% ¹³C-enriched N,N-dimethylformamide, under otherwise identical conditions to those reported by Patel. All of the ¹³C-enriched peaks (marked with asterisks) are assigned to N-formyl (N—CHO) groups.

FIG. 14 shows microscopy of mouse lungs 4 hours after nebulized delivery with the indicated polymers. Representative FISH microscopy images of aNLuc (white), Scgb1a1 and FoxJI markers of airway cells (magenta), and nuclei stained via DAPI (cyan). Scale bar represents 200 μm. n=2 mice per group.

FIG. 15 shows the percent mouse lung area positive for aNLuc RNA by FISH. Scale bar represents mean. n=2 mice per group.

FIG. 16A shows stability measurements using zeta potential of the polymers compared to hDD90-118. n=3 separate formulations, each of which was analyzed 3 times. *p<0.05, **p<0.01 (one-way ANOVA with multiple comparisons). Scale bars indicate mean±SD.

FIG. 16B shows polyplex size determination before and after nebulization using dynamic light scattering (DLS) of the polymers compared to hDD90-118. n=3 separate formulations, each of which was analyzed 2 times. *p<0.05, **p<0.01, ****p<0.0001 (Mixed-effects analysis). Scale bars indicate mean±SD.

FIG. 16C shows representative Cryo-electron microscopy (Cryo-EM) images of polymer P76 before and after nebulization. Scale bar represents 100 nm. Four representative particles are shown from 2 different particle preps from both pre- and post-nebulization samples. Molecular Dynamic (MD) simulation of P76 polymer unit with short RNA oligomer. Simulations were repeated 3 times.

FIG. 16D shows full frame images for the polymer P76 of FIG. 16C.

FIG. 17 shows representative whole frame cryo-EM images of P76 polyplexes before and after nebulization. Scale bars represent 100 nm. n=2 separate preparations per group.

FIG. 18 shows mass ratio results for P76. aNLuc mRNA was formulated with P76 at the indicated mass ratio. Polyplexes were nebulized at a dose of 25 μg/mouse and lungs were evaluated at 24 hours for aNLuc protein expression. The dotted line represents mean average radiance of the control group. Scale bars represent geometric mean±SD. n=3 mice per group. ****p<0.0001 by one-way ANOVA with Tukey's multiple comparisons on log-transformed data.

FIG. 19 shows quantification of different lengths of mRNA delivered to lungs. Cas13-2A-NLuc (4898 nt) or aNLuc (1298 nt) mRNA was formulated with P76 and delivered at 25 μg per mouse. Lungs were analyzed 5 minutes after delivery by qPCR for lung mRNA load. Scale bars represent geometric mean±SD. ns indicates p=0.2059 by unpaired two-tailed t-test of log-transformed data.

FIGS. 20A-20B show the biodistribution of P76 formulation expression in mice.

FIG. 20A shows images of the bioluminescence.

FIG. 20B shows average radiance of lungs, kidneys, heart, spleen, paraaortic lymph node, and liver 24 hours after aNLuc mRNA nebulized into mice at 5 mg/kg. n=4 mice per group. ****p<0.0001 (one way ANOVA with multiple comparisons on log-transformed data). Scale bars represent mean±SD.

FIG. 21 shows the distribution of P76 formulation expression in a cow lung. Images of other diaphragmatic lobe blocks from the cow were measured for luminescence 24 hours after aNLuc mRNA at 0.03 mg/kg. The two right images are of the same lobe section for clarity. Arrows indicate expression in larger cartilaginous airways.

FIGS. 22A-22B show P76 mediated delivery to human cell cultures. In FIG. 22A, the line represents mean of n=3 wells, and in FIG. 22B, the line represents the geometric mean of n=3 wells.

FIG. 22A shows human lung epithelial (A549) or human kidney epithelial (Expi293F) cells transfected with the indicated amount of green fluorescent protein (GFP) mRNA per well and analyzed for percent transfection.

FIG. 22B shows A549 or Expi293F cells transfected with the indicated amount of GFP mRNA per well and analyzed for protein expression.

FIG. 23 shows a representative whole section image of aNLuc mRNA FISH in macaque lung. The second image shows the macaque lungs 4 hours post transfection of 0.3 mg/kg aNLuc mRNA (white) using P76. Scgb1a1 and Foxjl airway markets (magenta) and nuclei (DAPI, cyan) for context. Scale bars represent 400 μm. n=1 macaque.

FIG. 24 shows a gel retardation assay of P76 and hDD90-118 binding of Cas13 mRNA and crRNA. Cas13 mRNA, crRNA, or a mixture was either left unformulated or was formulated with either P76 or hDD90-118 and loaded into a 1% agarose gel. Unformulated Cas13 mRNA and crRNA migration locations are indicated by the white box. This is a representative gel of 2 independent trials.

### DETAILED DESCRIPTION OF THE EMBODIMENTS

The present invention relates to methods of preventing or treating or controlling a respiratory disease in a bovine comprising:

delivering to a lung or trachea of the bovine a pharmaceutical composition comprising a pharmaceutically acceptable carrier and a polyplex,
wherein the polyplex comprises:
one or more, optionally further modified, mRNA encoded with a molecule selected from the group consisting of glycosylphosphatidylinositol (GPI) complex, antibody immunoglobulin G (IgG) antibody immunoglobulin A (IgA), antibody immunoglobulin M (IgM), antimicrobial peptide Bactenecin 5 (Bac5), antimicrobial peptide Bactenecin 7 (Bac7), bovine myeloid antimicrobial peptide (BMAP-28), lipoxin inducing enzymes, resolvin inducing enzymes, cytokines, CRISPR associated protein 13 (Cas13) enzymes, nanoluciferase (NLuc) proteins, and gene activator, catalytically dead CRISPR-associated protein 9 fused to an activator selected from the group consisting of VP64-p65-Rta transactivation domain (dCas9-VPR), VP64, and VP64-p65-HSF1 on the N terminus and SS18 on the C-terminus,
a polymer prepared by
a. polymerizing components i-ii and optionally iii:
   i. a diacrylate monomer;
   ii. one or more linker monomers including one or more functional groups independently selected from the group consisting of amino groups, alcohol groups and thiol groups; and
   iii. one or more branching monomers including one or more functional groups independently selected from the group consisting of amino groups, alcohol groups and thiol groups; and
b. end-capping the product of step (a) with one or more end capping monomers including one or more functional groups independently selected from the group consisting of amino groups and alcohol groups.

The terms “treating,” “treatment,” and “treat” refer to reversing, alleviating, delaying the onset of, or inhibiting the progress of a disease (e.g. a bacterial infection) described herein. In some embodiments, treatment may be administered after one or more signs or symptoms of the disease have developed or have been observed. In other embodiments, treatment may be administered to a susceptible subject prior to the onset of symptoms (e.g. in light of a history of symptoms and/or or in light of exposure to a pathogen). Treatment may also be continued after symptoms have resolved, for example, to delay and/or prevent recurrence.

The terms “prevent,” “preventing,” or “prevention” refers to a prophylactic treatment of a subject who is not and was not infected with a disease (e.g. a bacterial infection) but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

The terms “control,” or “controlling” refer to the ability to manage, alleviate, or reduce the severity, symptoms, or progression of a disease, with the goal of improving a subject's health or quality of life. In certain embodiments, it refers to measures taken to prevent the spread of the disease to others or to limit the severity of an outbreak.

The terms “composition” and “formulation” are used interchangeably.

The term “polyplex” refers to a complex comprising a polymer of the present invention and one or more agents. The polyplex may be in the form of particles, such as a nanoparticle. The one or more agents may include messenger RNA (mRNA), cargo RNA (cRNA), guide RNA (gRNA), transfer RNA (RNA), ribosomal RNA (rRNA), anchored RNA, and CRISPR RNA (crRNA).

The term “subject” to which administration is contemplated refers to an animal, such as a young or adult bovine, or any animal that belongs to the family Bovidae, which include domesticated and wild species of cloven-hoofed mammals. This includes, but is not limited to, cattle, such as the Bos Taurus, Bos Indicus, bison; and wild bovids, such as African buffalos, wildebeests, bantengs, yaks; hybrids, such as becfalo, and zubron.

The term “pharmaceutically acceptable carrier” may include, but is not limited to one or more excipients, solvents, emulsifiers, surfactants, stabilizers, pH adjusters, viscosity modifiers, sweeting agents, flavoring agents, coloring agents, and other miscellaneous additives for improving absorption, and other functional excipient for improving resorption.

The term “effective amount” of an agent as used herein, e.g., a pharmaceutical formulation, refers to an amount effective, at dosages and for periods of time necessary, to achieve the desired therapeutic or prophylactic result.

The term “Cas13-NL” refers to an mRNA encoding Cas13 fused with a glycine-serine (GS) linker to a nanoluciferase.

The term “Cas13-2A-NLuc” refers to a mRNA encoding Cas13 fused to nanoluciferase but with a P2A or T2A site. This causes the Cas13 and nanoluciferase to be separated during translation.

The term “aNLuc” is a GPI anchored version of nanoluciferase, meaning it will be linked to the plasma membrane, while NLuc is expressed in the cell cytosol.

The phrase “lipoxin inducing enzymes” may include 5-lipoxygenase (5-LOX), 15-Lipoxygenase (15-LOX), 5-Hydroxyeicosanoid Dehydrogenase (5-HEDH), Lipoxin A4 Synthase (ALX/FPR2), Leukotrienc A4 Hydrolase (LTA4H), and 15-Oxo-Prostaglandin 13-Reductase (15-PGDH).

The phrase “resolving inducing enzymes” may include 5-lipoxygenase (5-LOX), 15-Lipoxygenase-1 (15-LOX-1), 15-Lipoxygenase-2 (15-LOX-2) 5-Hydroxycicosanoid Dehydrogenase (5-HEDH), Leukotriene A4 Hydrolase (LTA4H), 15-Oxo-Prostaglandin 13-Reductase (15-PGDH), and 5-Lipoxygenase Activating Protein (FLAP).

The flexibility of nucleic acid-based drugs allows a variety of therapeutic proteins to be encoded for use as a treatment, prophylactic, or vaccine. Efficacious delivery of cargo to a target organ enables the development of multiple therapeutic programs with minimal vehicle redesign requirements. Given the large number of pulmonary pathogens and diseases including SARS-COV-2, influenza, and cystic fibrosis (CF), among others, efficient delivery of messenger RNA (mRNA) to the lungs represents a promising approach for both treatment and prevention.

To improve pulmonary delivery of PBAE formulations, the present invention employs a combinatorial synthesis strategy with a low dead-volume, nebulizer-based particle screening system to assess PBAEs and PBAE-containing formulations in mice. Several useful polymers were developed. Polymer P76, a poly-beta-amino-thio-ester (PBATE) enabled potent delivery of mRNA, regardless of cargo size and complexity, to mice, hamsters, ferrets, cows, and rhesus macaques. P76 was found to be safe and well-tolerated, with greater expression than previous nebulized PBAE polymers. Polymer P76 allowed for 4-fold dose sparing, when compared with previous nebulized PBAEs, in a Cas 13a efficacy study against SARS-COV-2 in the Syrian hamster model. This approach was also shown to be competitive with high doses of intraperitoneal delivered neutralizing antibodies.

As used herein a “polyplex” refers to a polymeric system containing condensed and/or complexed RNA through electrostatic interactions between cationic groups of the polymer and the negatively charged nucleic acids.

Functional Screening of Polymers for Nebulized mRNA Delivery

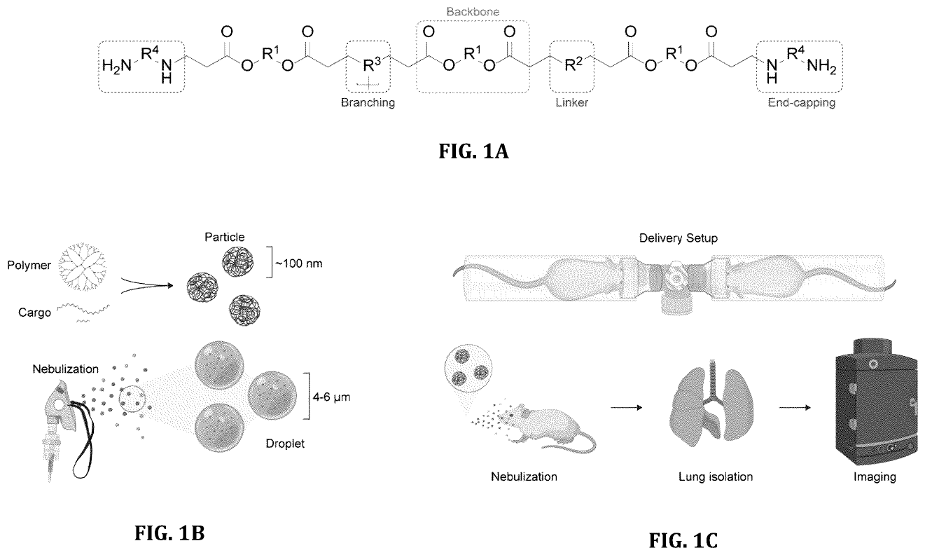

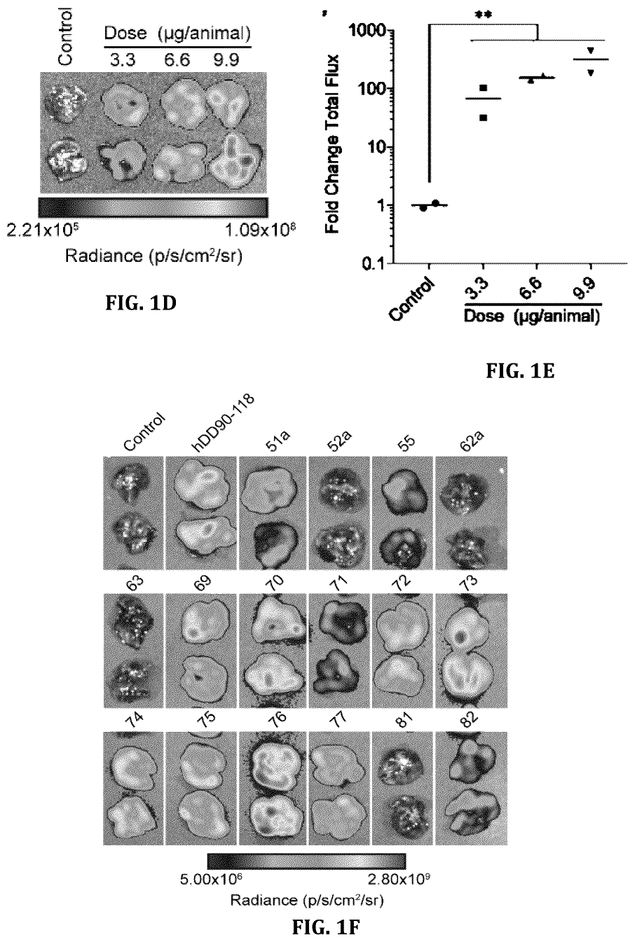

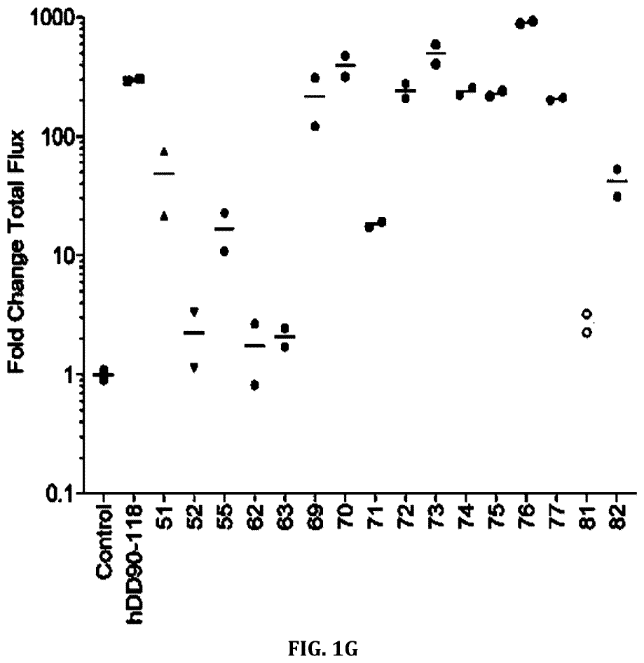

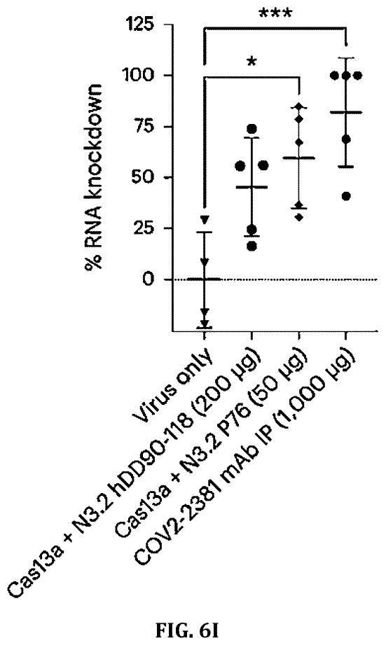

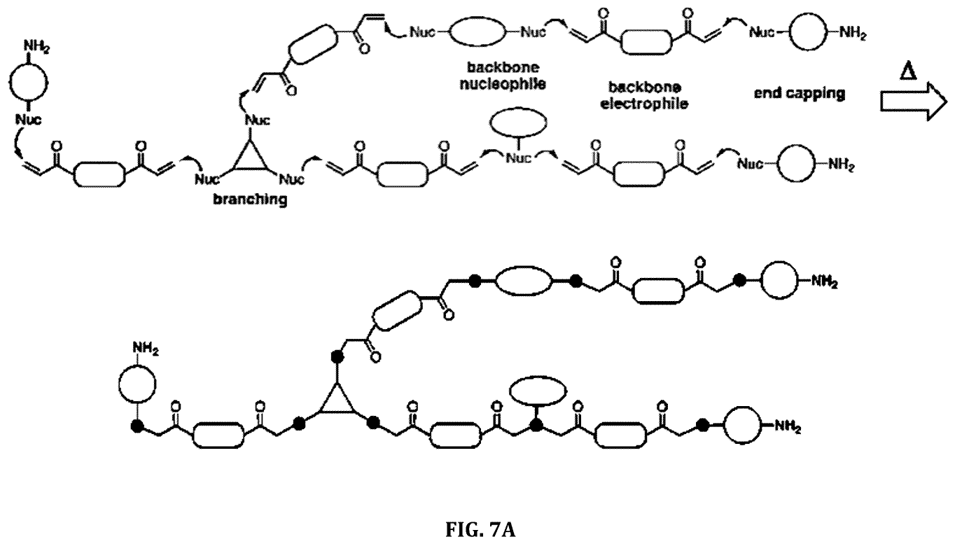

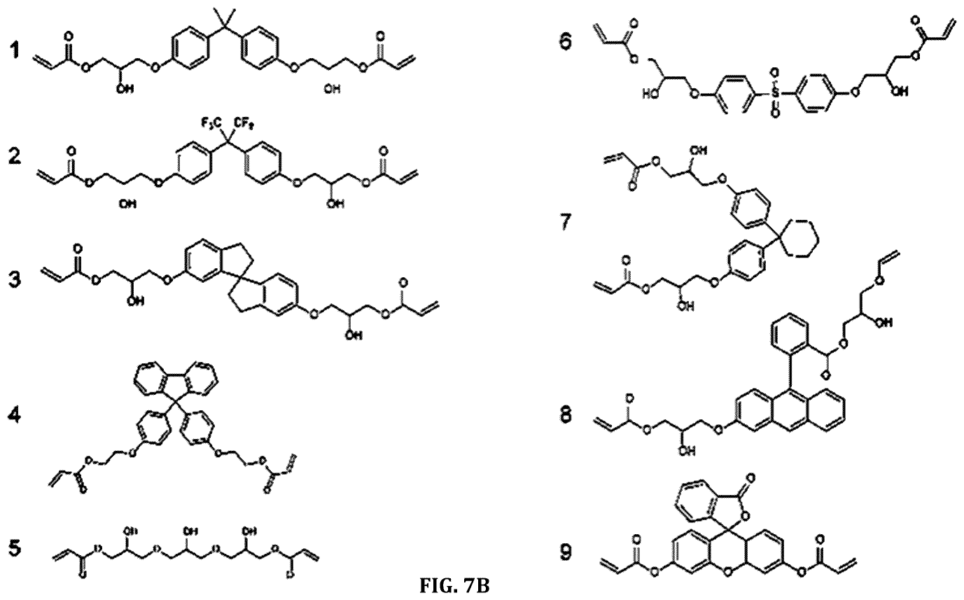

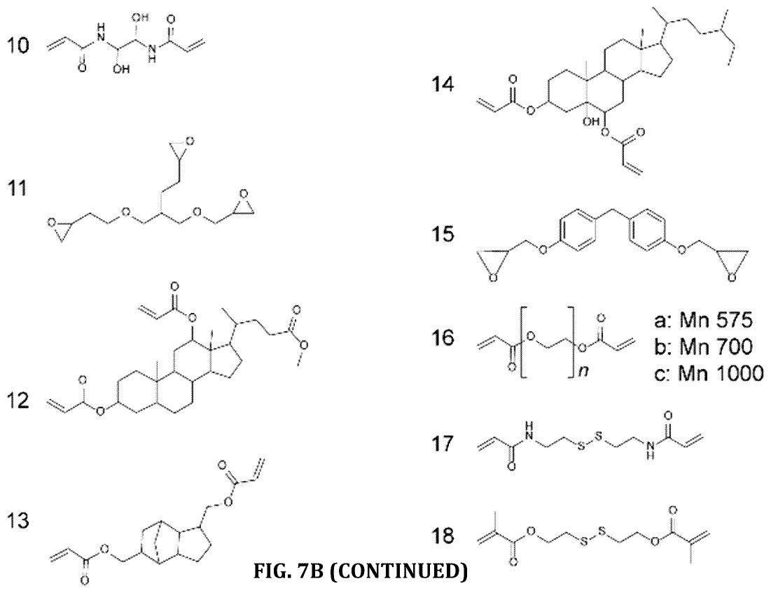

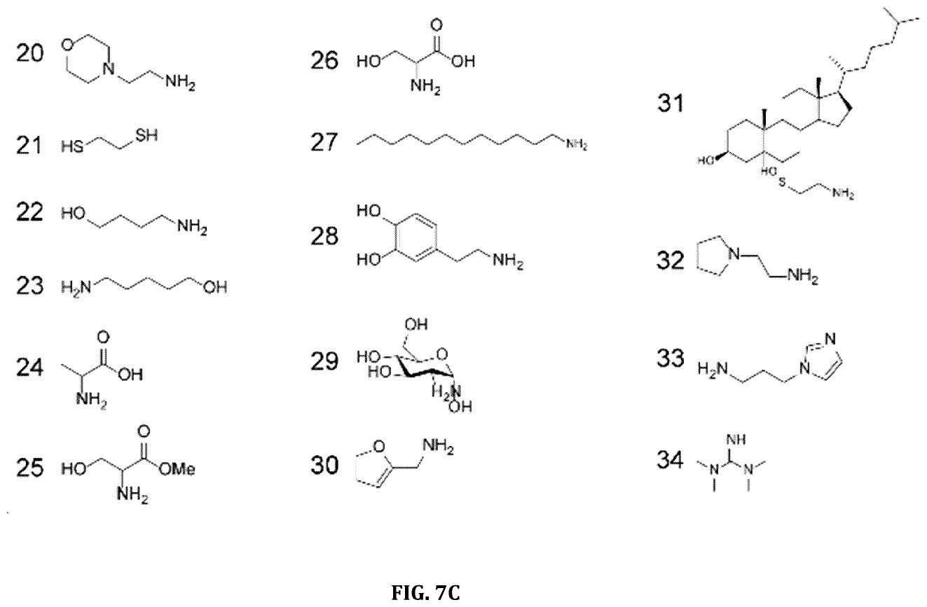

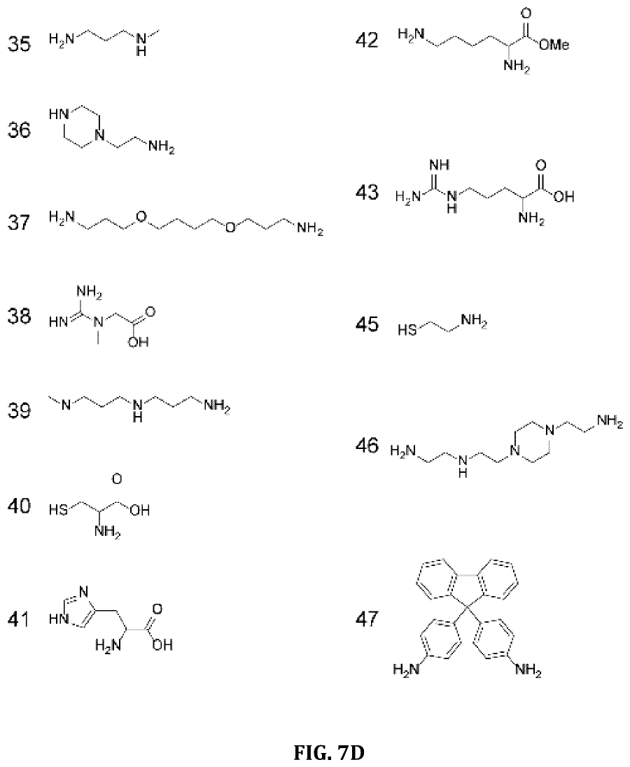

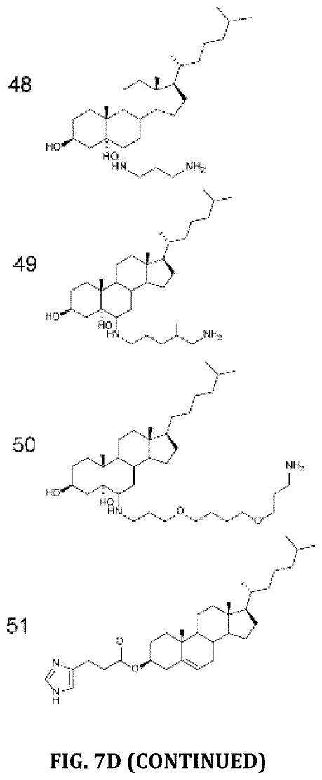

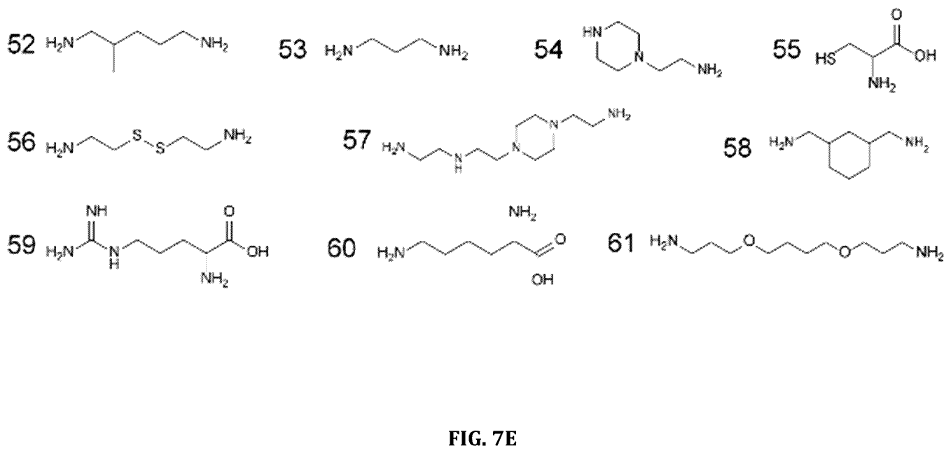

Using the hDD90-118 PBAE as a starting point, a set of polymers for mRNA delivery to the respiratory track via nebulizer were made (FIGS. 7A-7E and FIGS. 8A-8B and Table 1).⁹ These polymers included a fundamental structure consisting of: 1) diacrylate backbone electrophilic components, 2) multifunctional amines, amino alcohols, and amino thiols as linear or branching nucleophilic components, and 3) diamine or aminothiol components added in excess after an initial polymer-forming step to place amine groups at chain ends (FIG. 1A, and FIGS. 7A-7E). In addition to the bisphenol glycerol diacrylate used in hDD90-118, polymers and copolymers containing diacrylates derived from a variety of other building blocks including glycidyl ethers, xanthene dyes, and steroids, as well as several methacrylate, acrylamide, and epoxide derivatives were synthesized and tested.

The polymers were tested exclusively in pH 5 sodium acetate buffer to retain protonation of the polymer, facilitating RNA binding. When delivered via a vibrating mesh nebulizer, the nanoparticle colloid was aerosolized, with a droplet diameter on the order of 4-6 μm for enhanced drug delivery to the deep lung (FIG. 1B).^{11,12}

In order to perform a functional screen, polyplexes of each polymer with mRNA encoding a glycosylphosphatidylinositol (GPI)-anchored nanoluciferase (aNLuc) reporter were formulated; hDD90-118 was used as a positive control.¹⁰ Polyplexes containing aNLuc mRNA were then tested using a vibrating mesh nebulizer attached to a custom nose-only apparatus, evaluating functional in vivo delivery via nanoluciferase expression (FIG. 1C). The nose-only nebulizer apparatus design minimizes the dead volume (the space between the nebulizer outlet and the inhaling region of the animals), allowing for doses as low as 0.165 mg/kg (3.3 μg/mouse) of aNLuc mRNA (FIG. 1D and FIG. 1E). In addition, the apparatus exhibited minimal animal-to-animal variability in expression, allowing for the use of only 2 animals per formulation.

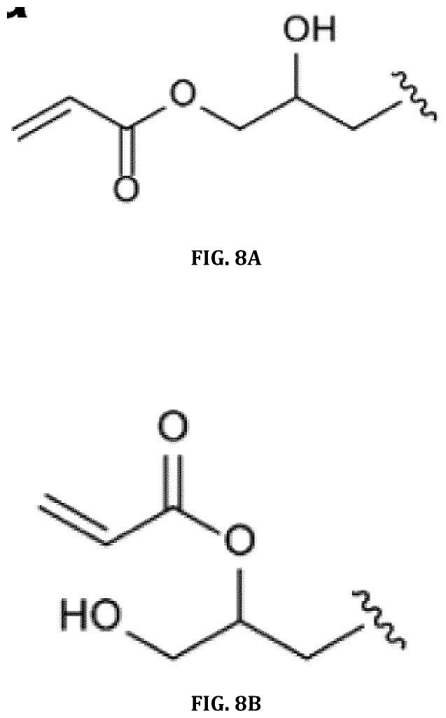

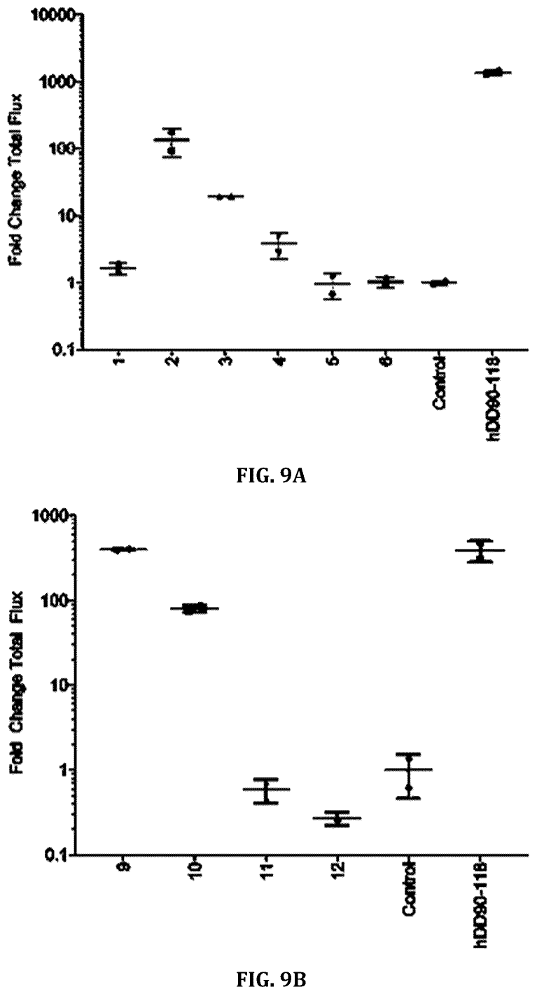

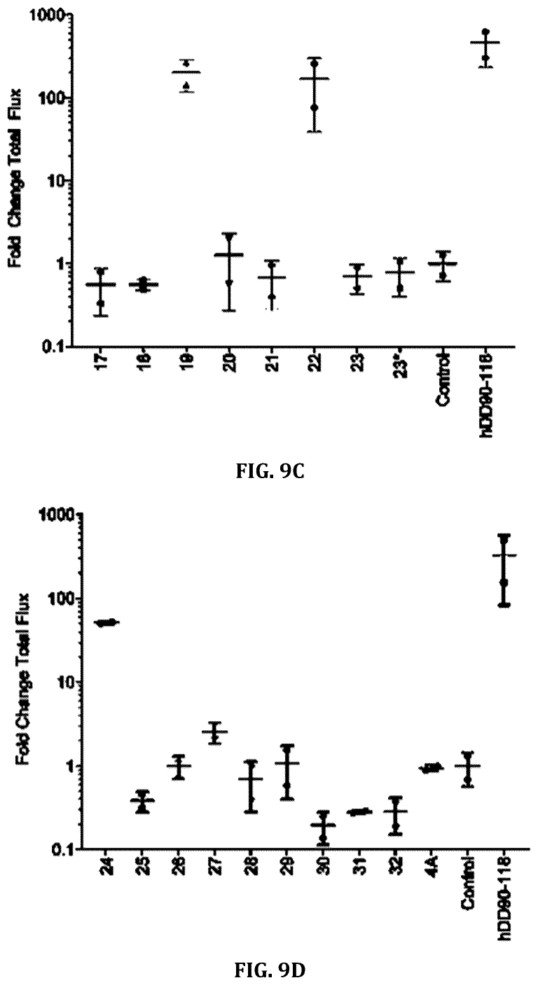

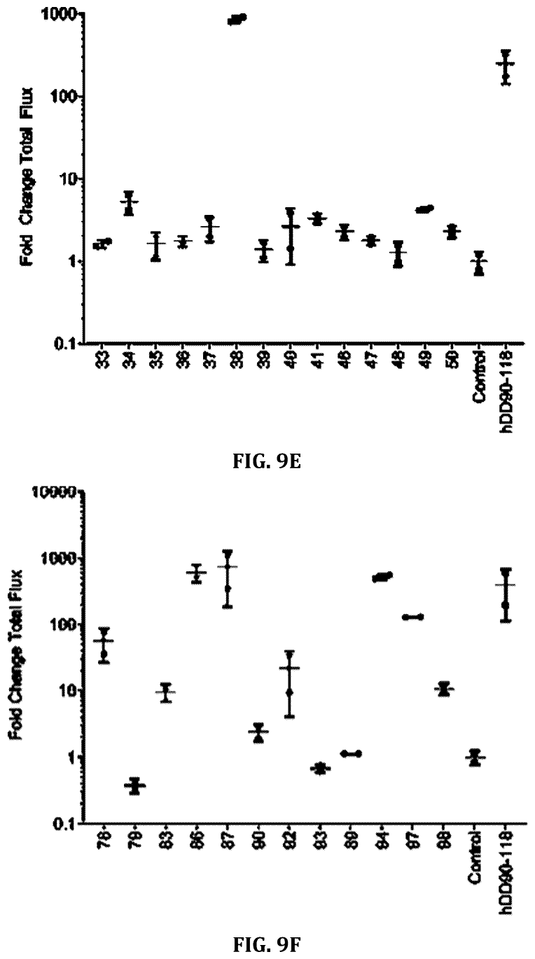

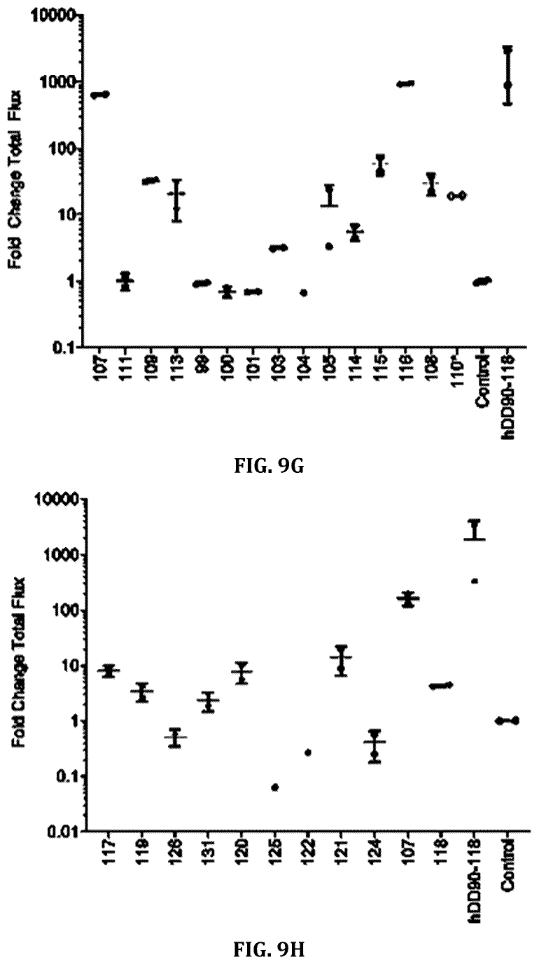

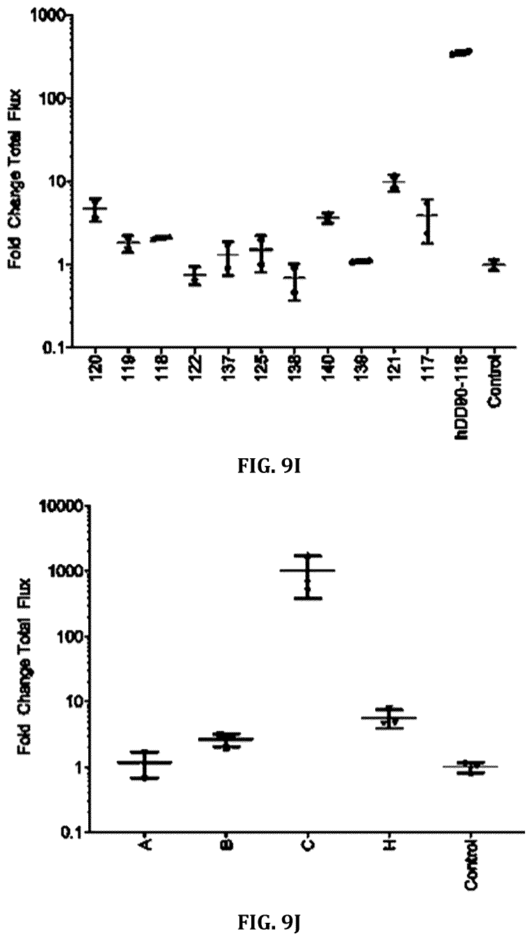

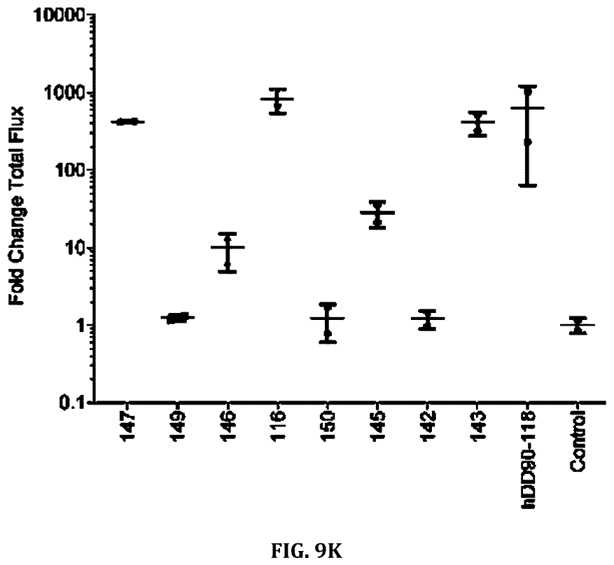

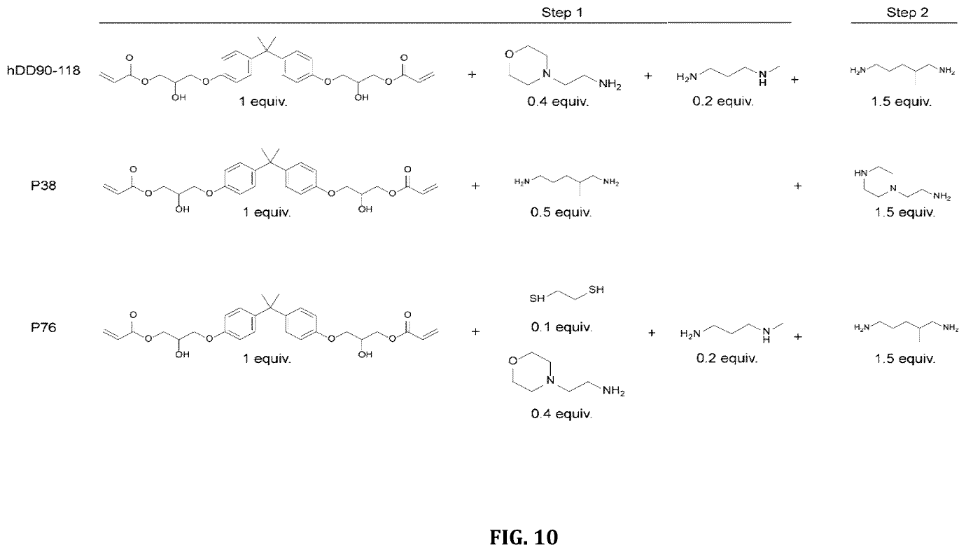

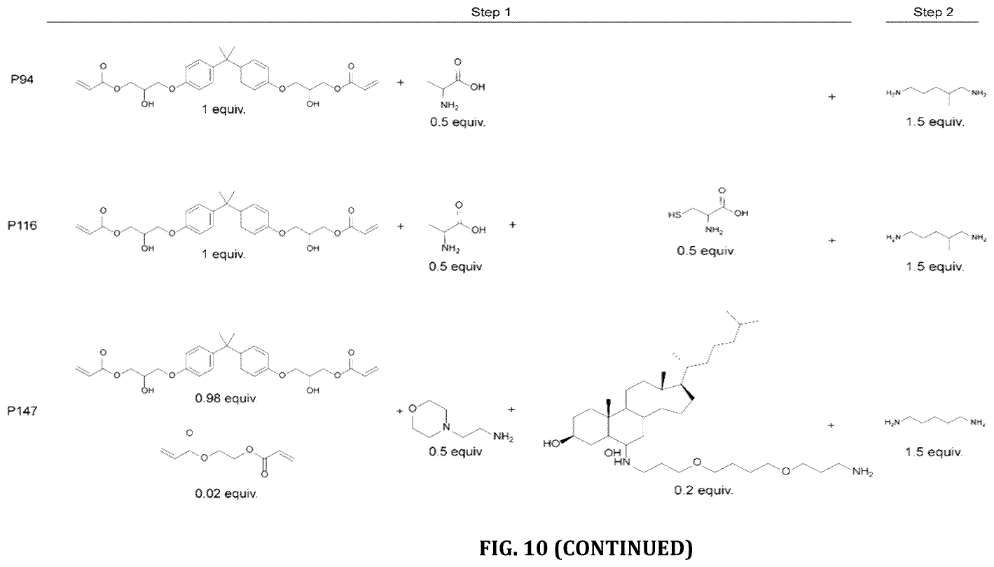

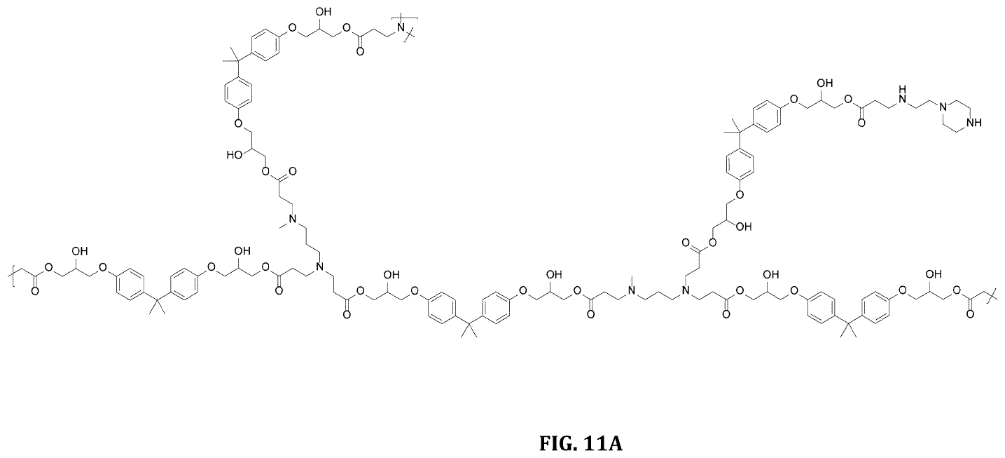

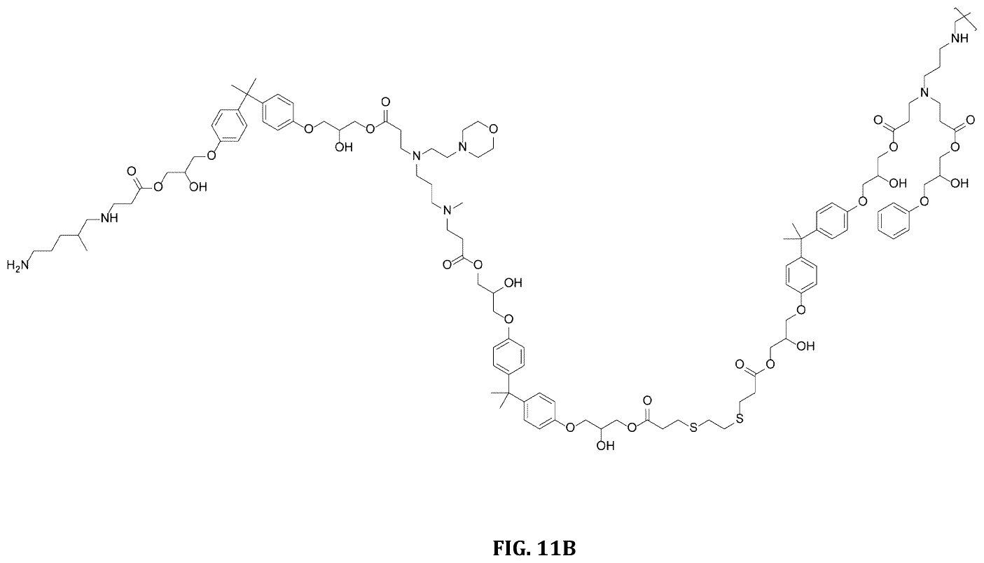

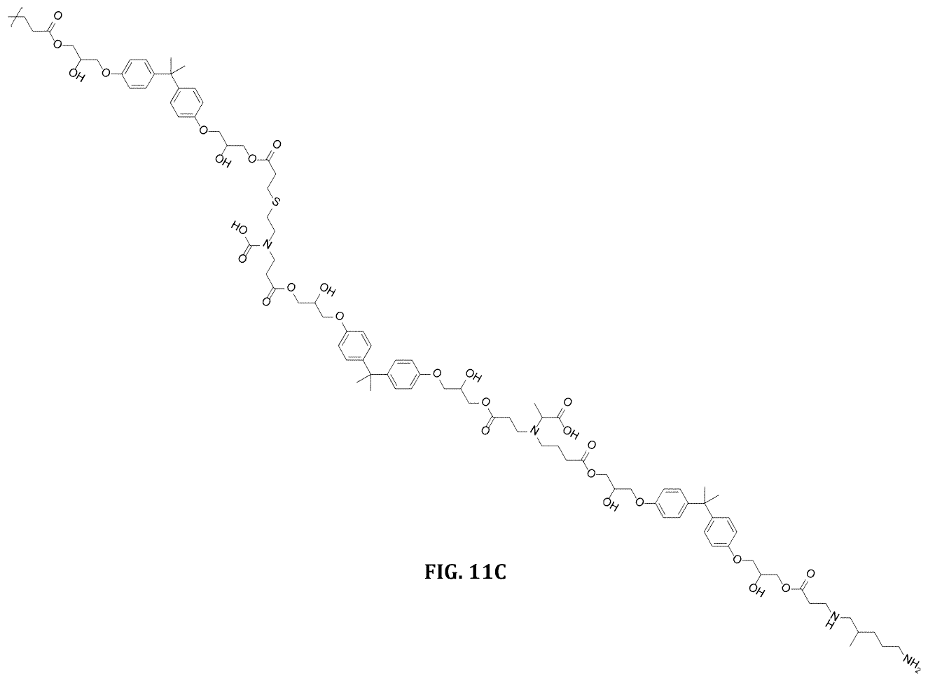

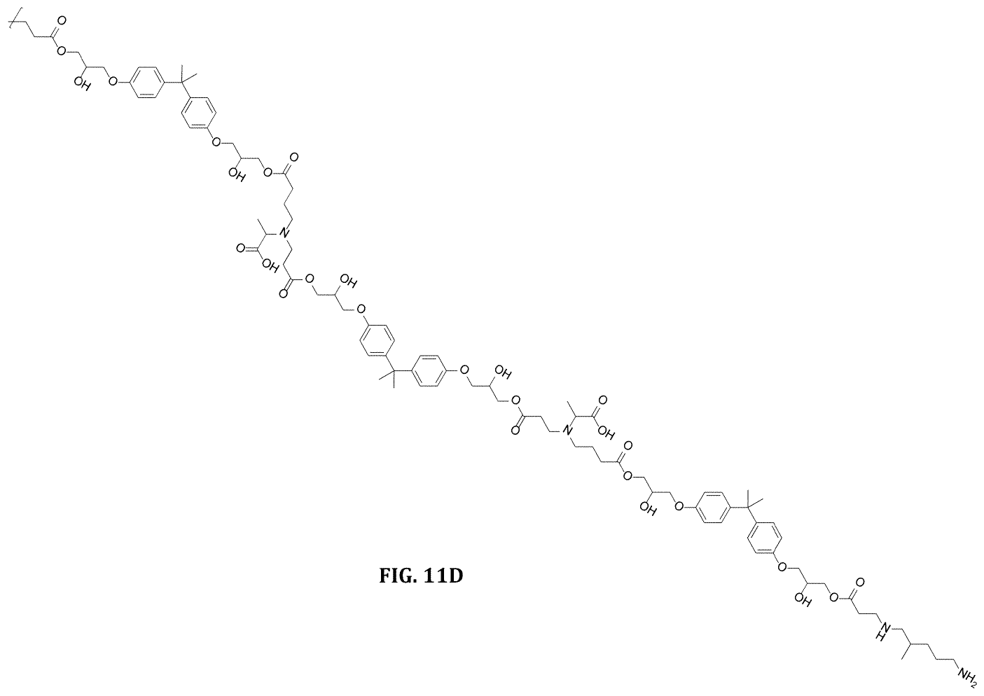

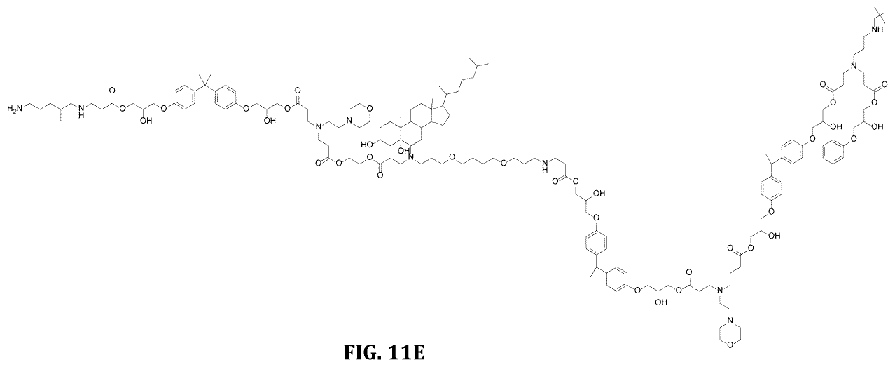

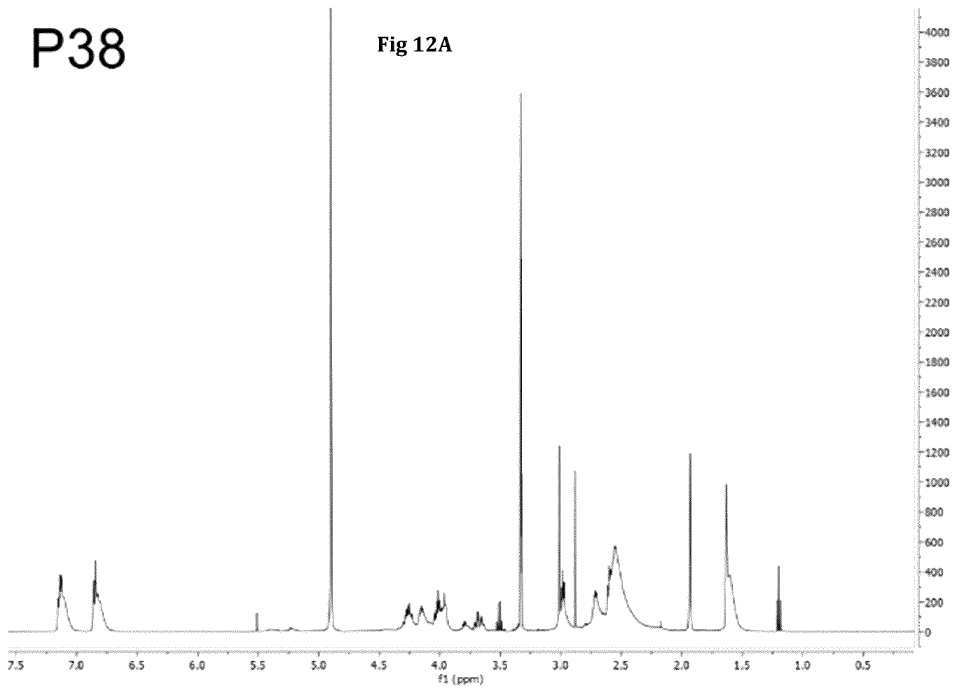

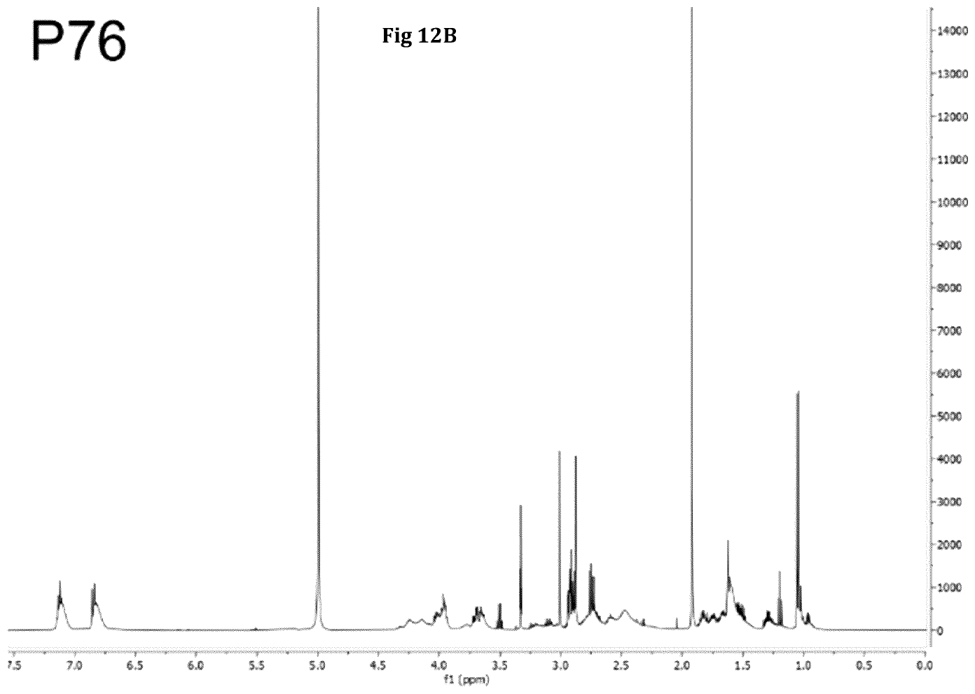

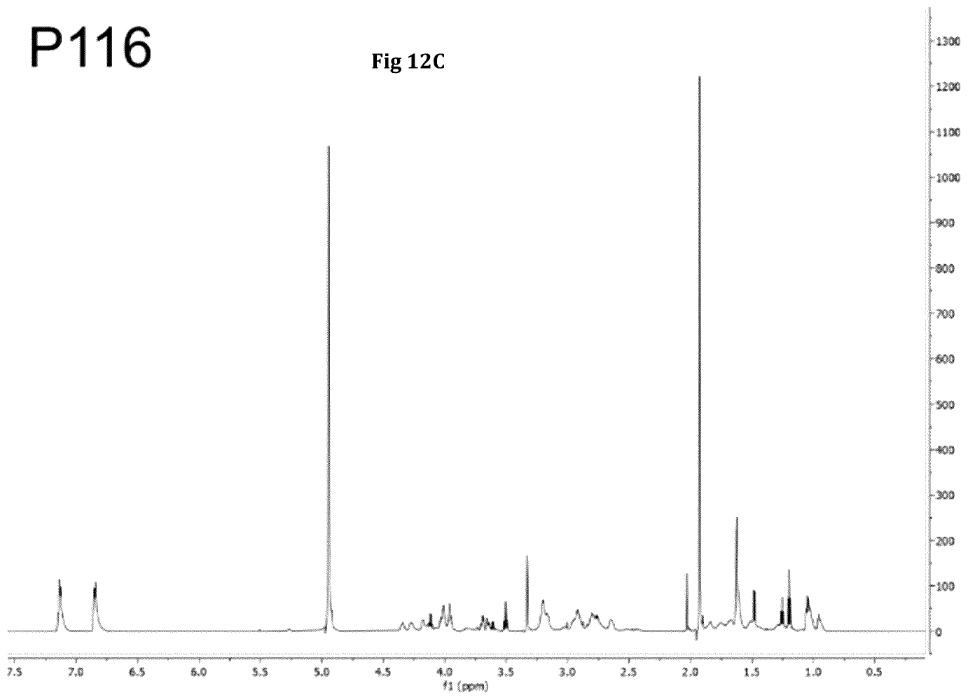

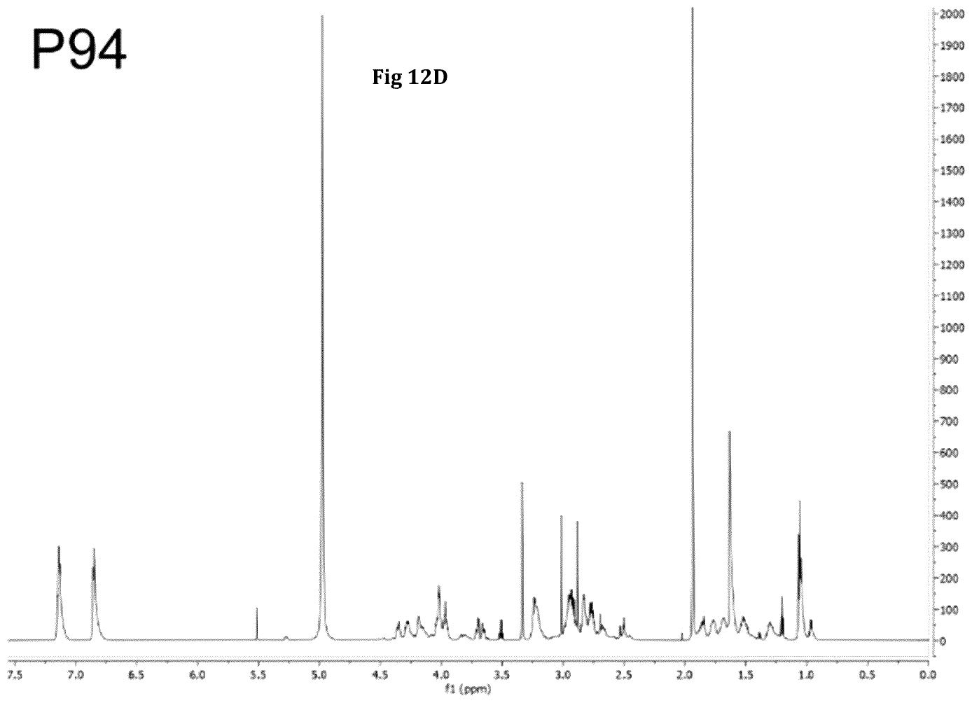

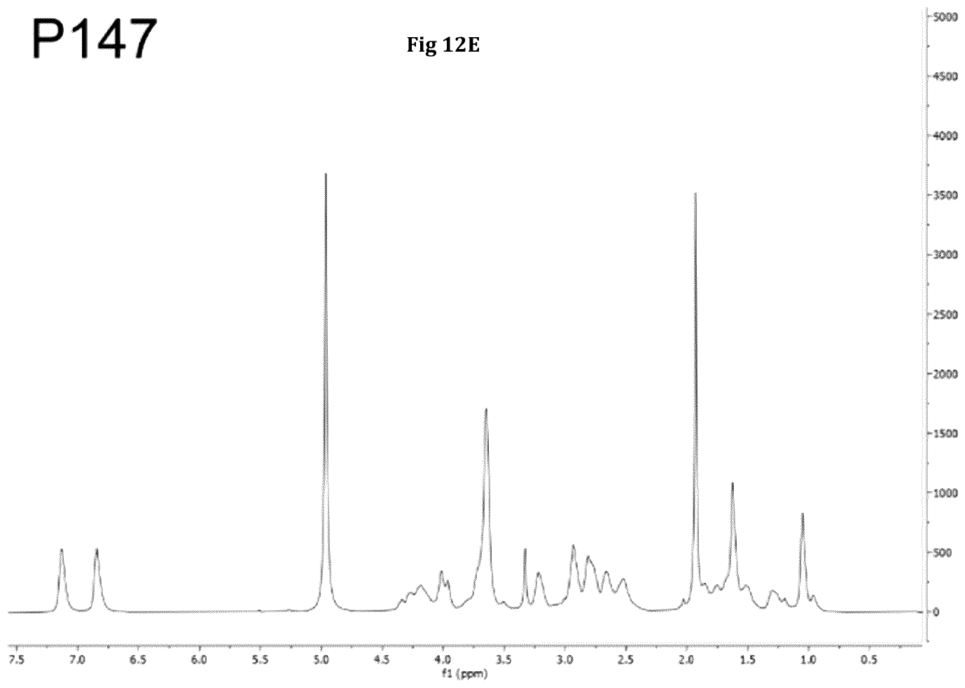

Five polymers (P38, P76, P94, P116, and P147) emerged from the initial screen with expression as high or higher than hDD90-118 (FIG. 1F, FIG. 1G, Table 2, and FIG. 9-FIG. 12). In some of these polymers, cysteamine or 1,2-ethanedithiol was incorporated as nucleophilic main-chain building blocks, which add on to acrylates much faster than amines. P76, which incorporated dithiol and was designated as a PBATE, exhibited a 3.02-fold higher expression when compared with hDD90-118.

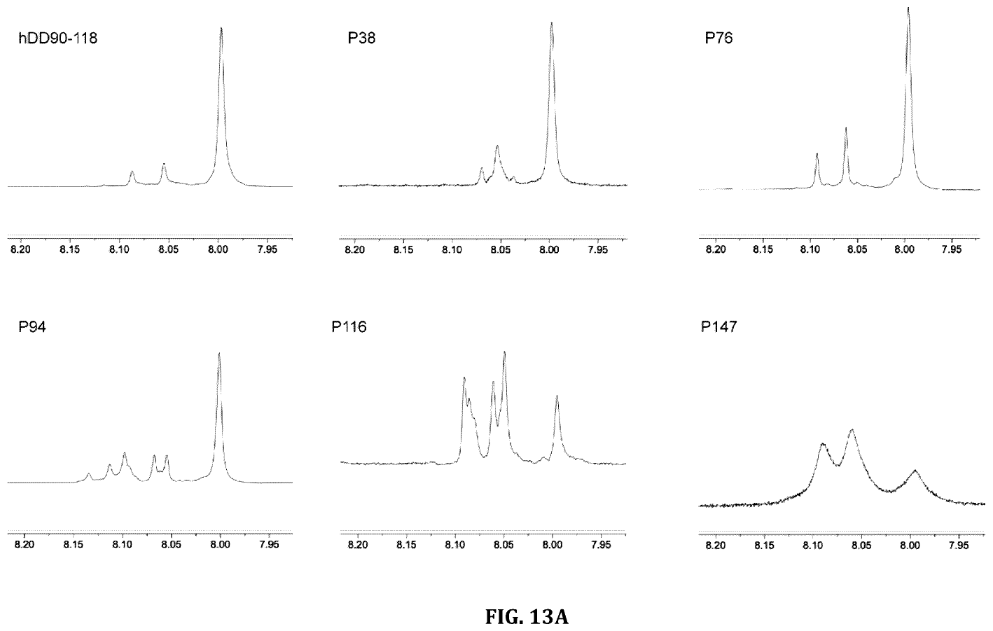

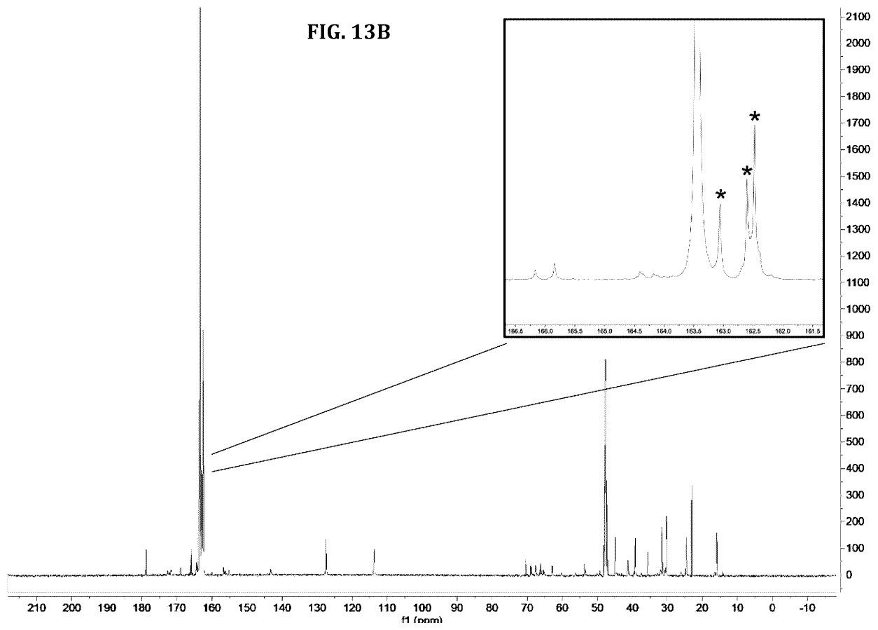

The dithiol was particularly interesting, since small amounts reacted quickly with bis (acrylates), creating macromonomer or block-oligomer-like components in situ. In addition, NMR analysis of hDD90-118 and the 5 polymers revealed the presence of peaks in the 8 ppm-8.15 ppm range, which was unexpected based on the previously reported composition (FIGS. 13A-13B).⁹ Given the reaction conditions described (heating in N,N-dimethylformamide solvent at 90° C. for 48 hours), N-formylation was likely, since dimethylformamide (DMF) is known to be a potential donor of the formyl group.¹³⁻¹⁸ The carbonyl groups derived from the solvent were confirmed by preparation of hDD90-118 in the presence of ¹³C-enriched DMF (FIG. 13A-13B). Delivery of reporter mRNA to mice lungs using hDD90-118 made in a different solvent (N,N′-dimethylpropyleneurea (DMPU), Polymer 98) was much lower than hDD90-118 made using DMF, suggesting that N-formylation may be a key attribute (FIG. 9).

Overall, these results demonstrate that the nebulizer apparatus of the present invention, when used in conjunction with a sensitive luminescent reporter construct, is ideal for identifying polymer formulations for delivery of mRNA in vivo.

### Chemical Analysis and Performance of the Polymers

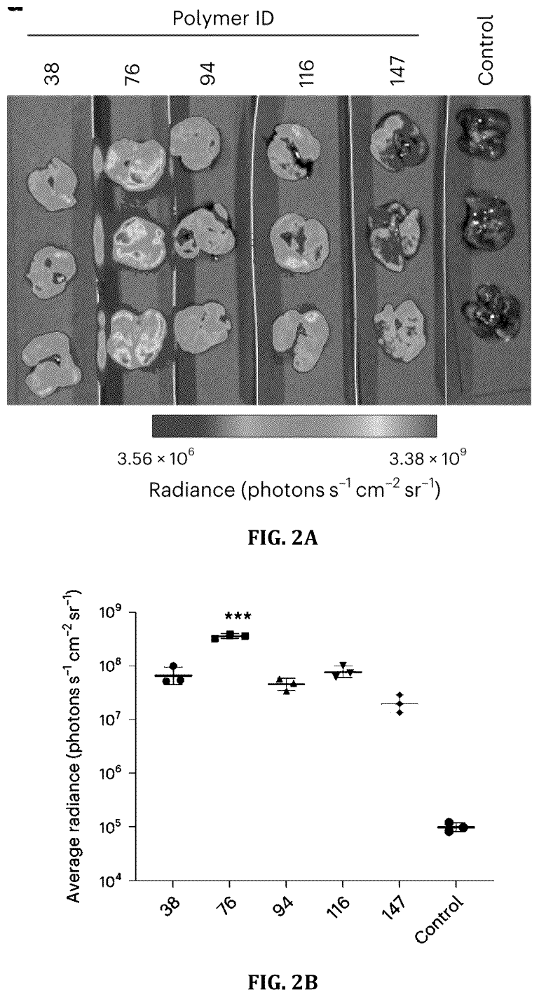

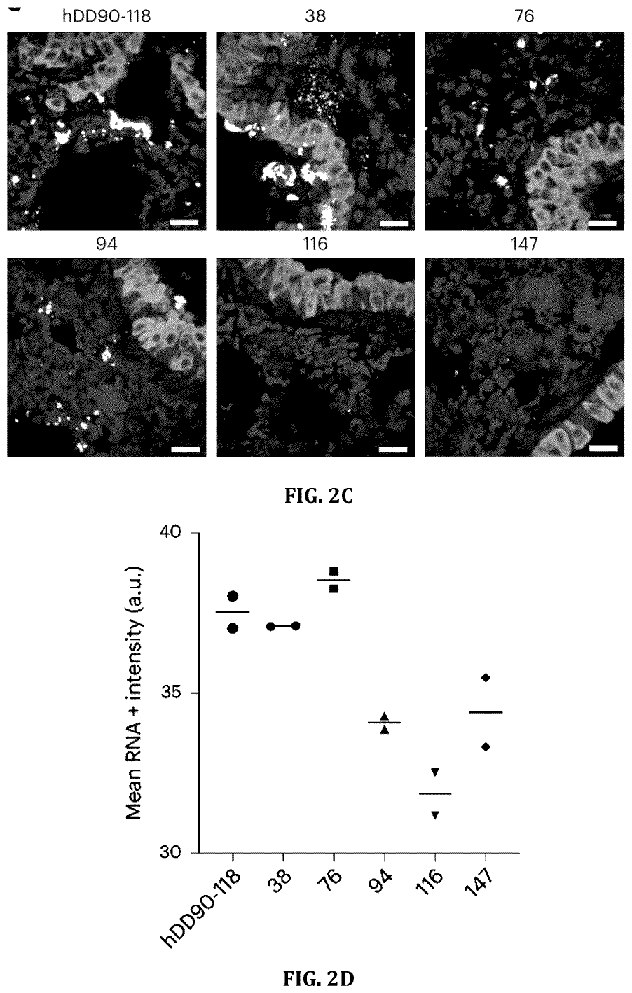

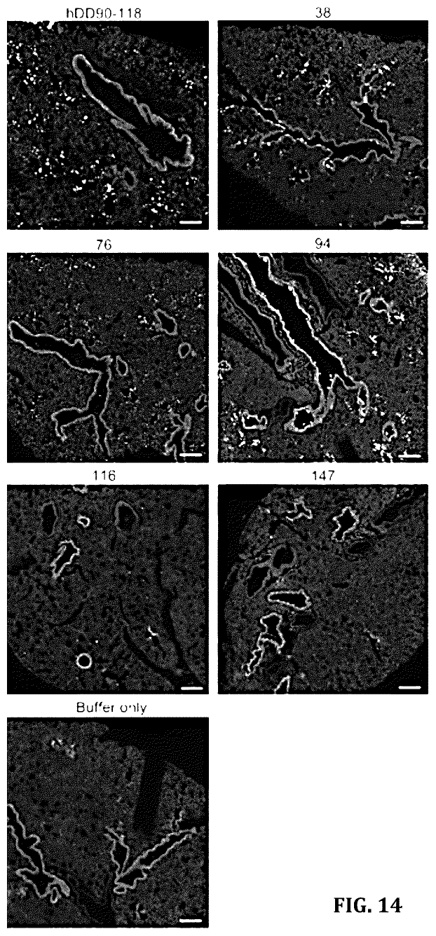

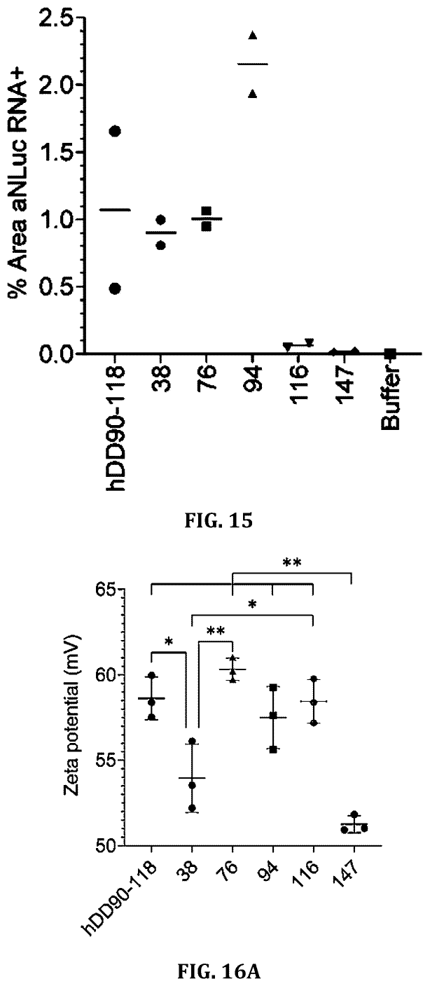

P38, P76, P94, P116, and P147 were compared using a variety of assays. First, the expression of a 5 mg/kg dose of aNLuc mRNA formulated with the polymers in the lungs of mice (FIG. 2A) was compared. Delivery with the P76 PBATE resulted in a geometric mean lung radiance of 3.58×10⁸ p/s/cm²/sr, significantly higher (by 5.46×, 7.88×, 4.63×, and 18.12×) than P38, P94, P116, and P147, respectively (FIG. 2B). Next, the distribution of mRNA cargo delivered by the polymers along with hDD90-118 in the lungs of mice was analyzed. Fluorescent in situ hybridization (FISH) microscopy revealed that P38, P76, P94, and hDD90-118 delivered mRNA to the lung, with P38 and P94 delivery resulting in some deposition of RNA in the airways, while P76 and hDD90-118 delivery produced more alveolar RNA signal (FIG. 2C, FIG. 14). Quantification of FISH signal revealed that the percent of RNA-positive lung area does not correlate strongly with expression (FIG. 15). However, P76, P38, and hDD90-118 formulations resulted in slight increase in the mean intensity of RNA positive regions, correlating with higher aNLuc expression (FIG. 2D).

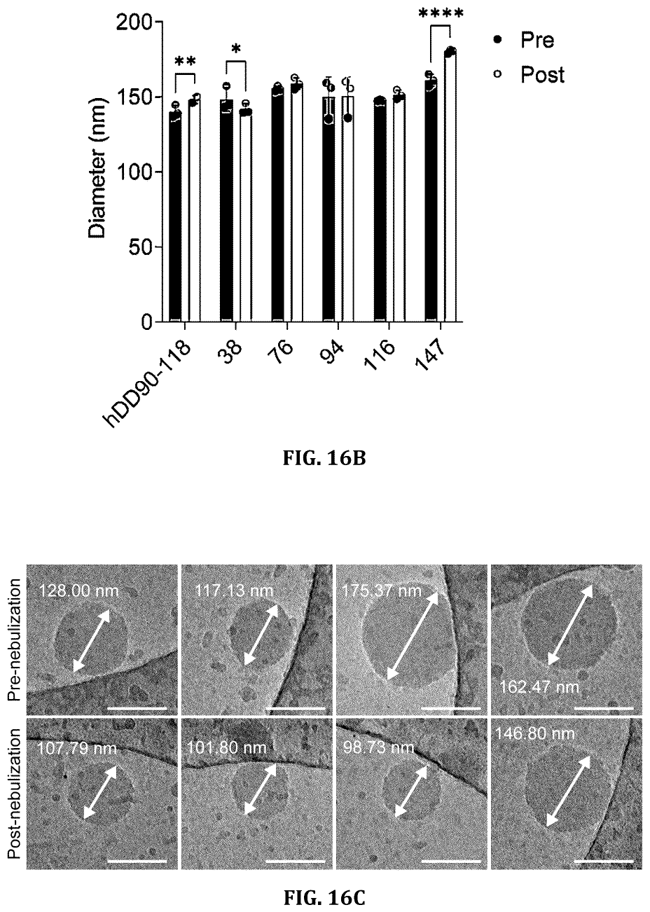

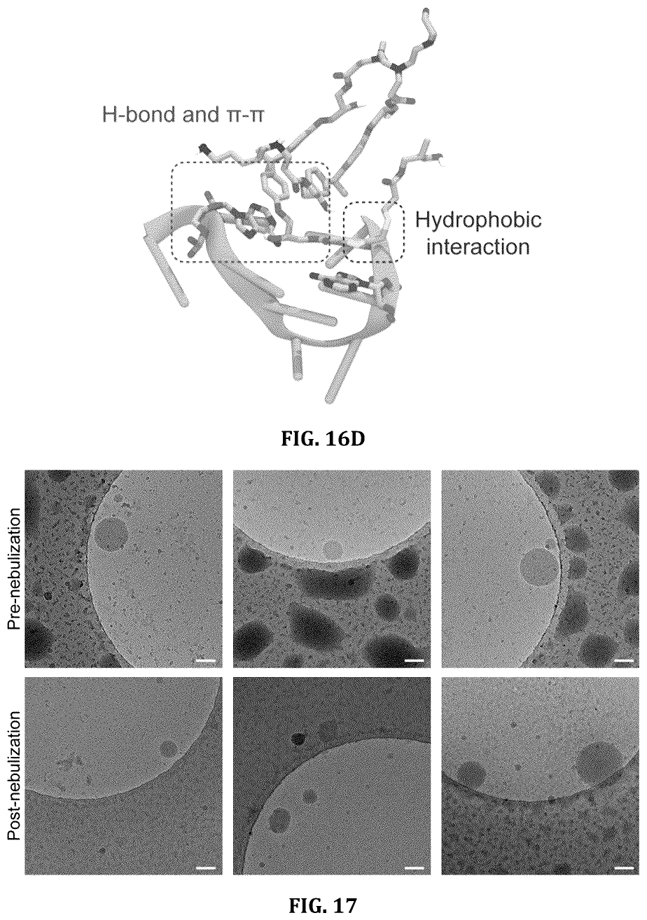

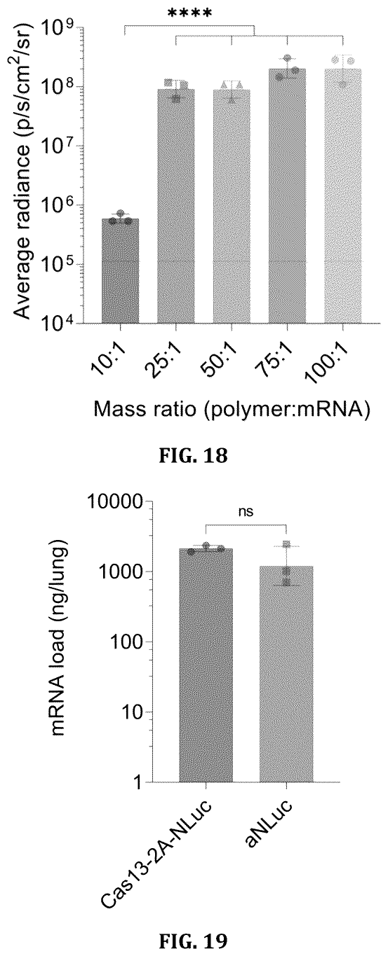

All particles exhibited highly positive surface zeta potentials and favorable diameters before and after nebulization (FIGS. 16A-16D & FIG. 17). In addition, molecular dynamics simulations were performed to determine the interaction of P76 with mRNA (FIG. 16). Finally, a wide range of mass ratios, between 10:1 and 100:1, were measured finding minimal changes in expression of aNLuc mRNA, with the exception of the 10:1 ratio (FIG. 18). Thus, the 50:1 mass ratio that was used previously with hDD90-118 was selected.⁹

Nebulized Delivery of a Variety of mRNA Cargos Using P76

The ability of P76 to deliver mRNA cargos of various lengths and complexity to the lungs of mice was then determined. mRNA encoding an immunoglobulin G (IgG) antibody was delivered, (light chain mRNA length: 1,890 nt, heavy chain mRNA length with encoded GPI anchor: 2,180 nt), anchored human contraceptive antibody (aHCA), with no target in non-human species, LbuCas13a (4,719 nt), an activatable RNase, or the gene activator, dCas9-VPR (˜7,055 nt). For both Cas13a and dCas9-VPR, a CRISPR RNA (crRNA) of 60 nt for Cas13a and 100 nt for dCas9-VPR was included, to simulate a CRISPR drug.

Suitable examples of molecules used for encoding the mRNA may include Cas13 enzyme variants. Suitable examples of Cas13 enzyme variants may include but are not limited to Cas13a, the original Cas13 enzyme variant discovered in the bacterium Leptotrichia shahii; Cas13b, an enzyme discovered in Prevotella sp. P5-125; Cas13c, a Cas13 enzyme variant that was recently discovered in the bacterium Bacteroides sp. AAC00246, Cas13d, a Cas13 enzyme variant that was recently discovered in the bacterium Ruminococcus sp. CAG_599; Cas13e, a Cas13 enzyme variant in the bacterium Porphyromonas gingivalis, Cas13f, a Cas13 enzyme variant in the bacterium Bacteroides thetaiotaomicron, LbuCas13a, a Cas13a enzyme variant in the bacterium Leptotrichia buccalis; CasRx, a Cas13 enzyme variant in the bacterium Prevotella buccalis; and Cas13×, a Cas13 enzyme variant that was in the bacterium Ruminococcus champanellensis.

To assess IgG expression, an mRNA-encoded light chain fused to the NLuc protein (aHCA-Nluc) was used, while for the Cas 13a and dCas9-VPR constructs, the NLuc reporter sequence was downstream of an encoded P2A cleavage site.¹⁹ The difference in expression was also checked using P76 between BALB/c and DBA/2 mice strains since strain-dependent differences in transfection efficiency have been previously reported.²⁰

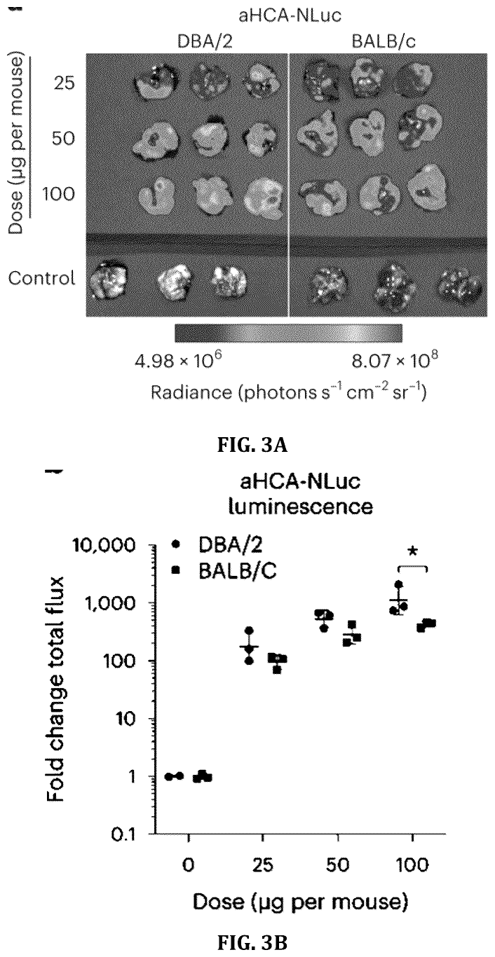

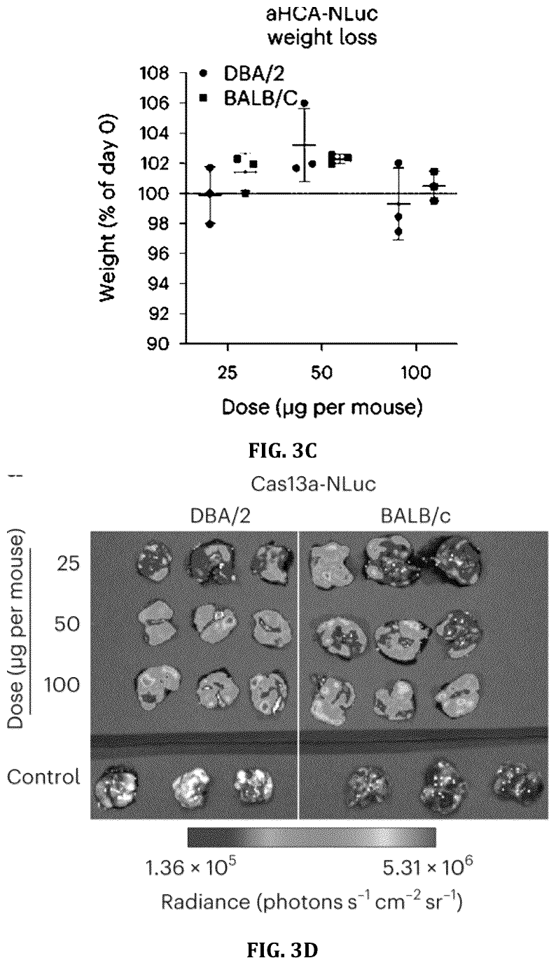

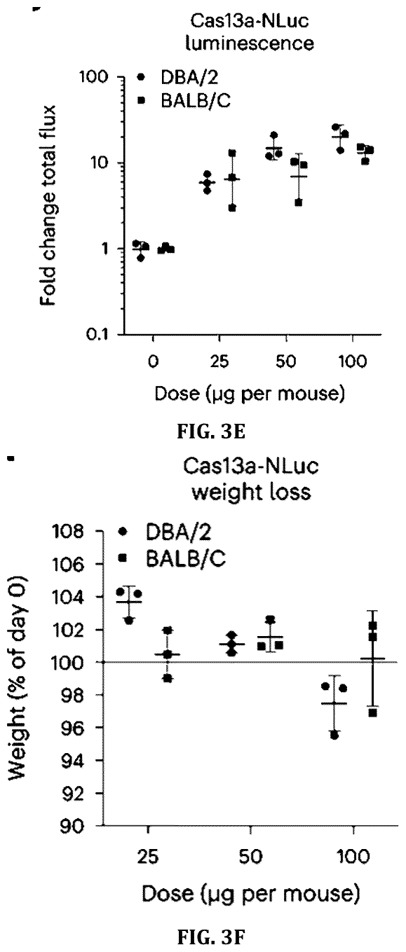

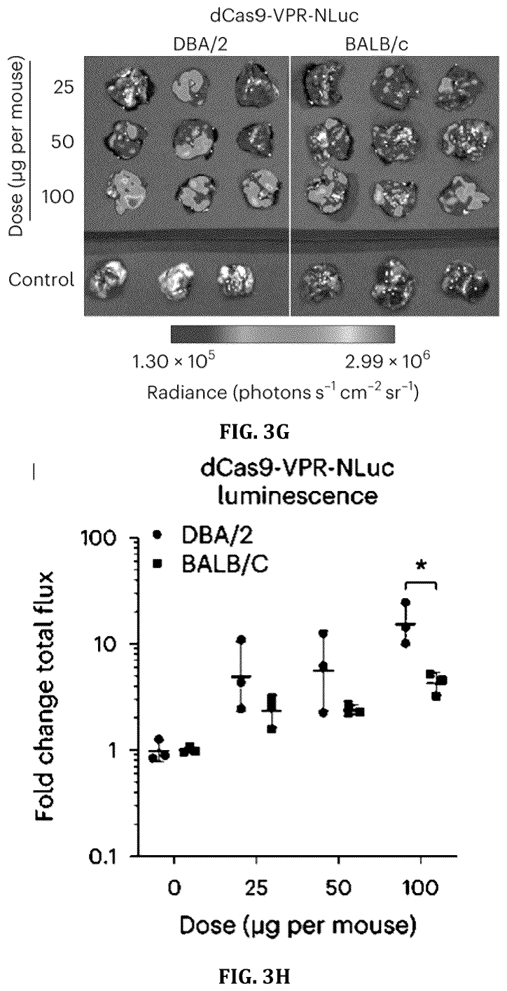

First, when mRNA encoding aHCA-NLuc was administered via nebulizer, DBA/2 mice displayed an increase in geometric mean signal fold-change compared to BALB/c mice at the tested dosages of 1.25, 2.5, and 5 mg/kg (FIGS. 3A-3B). This increase in signal was 2.10 times higher in the DBA/2 mice. Neither mouse strain displayed any significant weight change after 24 hours, a clinical metric of general health, at any of the tested dosages of aHCA-NLuc mRNA (FIG. 3C). When mice were delivered the same dosages above of Cas13a-NLuc along with a crRNA, DBA/2 mice produced a signal 1.52 times higher than BALB/C on average, resulting in a small but measurable drop in weight that increased according to dose (FIG. 3D-3F). Finally, dCas9-VPR-NLuc mRNA delivered via P76 to DBA/2 mice exhibited a signal 2.69 times higher than BALB/c mice on average (FIG. 3G-3H). Similar to the Cas13a-NLuc results, mice who received dCas9-VPR-NLuc mRNA, delivered with crRNA, exhibited a small but measurable drop in weight that increased according to dose (FIG. 3I). Overall, these results demonstrate that P76 can deliver cargo across a wide range of sizes and confirmed that DBA/2 mice consistently demonstrated slightly higher expression than BALB/c mice. The slight increase in protein expression in the DBA/2 mice is likely due to a combination of increased inspiratory duty cycle of 19% over the BALB/c strain and inter-strain differences in lung anatomy. ^{21,22}

To confirm that P76 efficiently delivers mRNA of various lengths, 25 μg of either aNLuc (1298 nt) or Cas 13-NLuc (4898 nt) was delivered to the lungs of mice via nebulizer and the amount of RNA delivered to the lungs was analyzed by quantitative PCR. No statistical difference was found in the mass of delivered mRNA despite the 3.77 times difference in mRNA length, indicating that P76 delivers cargos of different lengths with similar efficiency (FIG. 19). Considering that 1) the increased size of the Cas13 and VPR constructs results in fewer RNA copies delivered at the same dosage compared to the anchored antibody construct, 2) the membrane anchor retains the antibody in the tissue and increases NLuc signal^{2, 19, 23}, and 3) the 80-95% efficiency loss of NLuc expression due to the P2A cleavage site²⁴, the signal observed in mice lungs transfected with the Cas13 and VPR constructs had similar expression to the antibody construct when the above factors were taken in to account.

### Nebulized P76 is Minimally Toxic

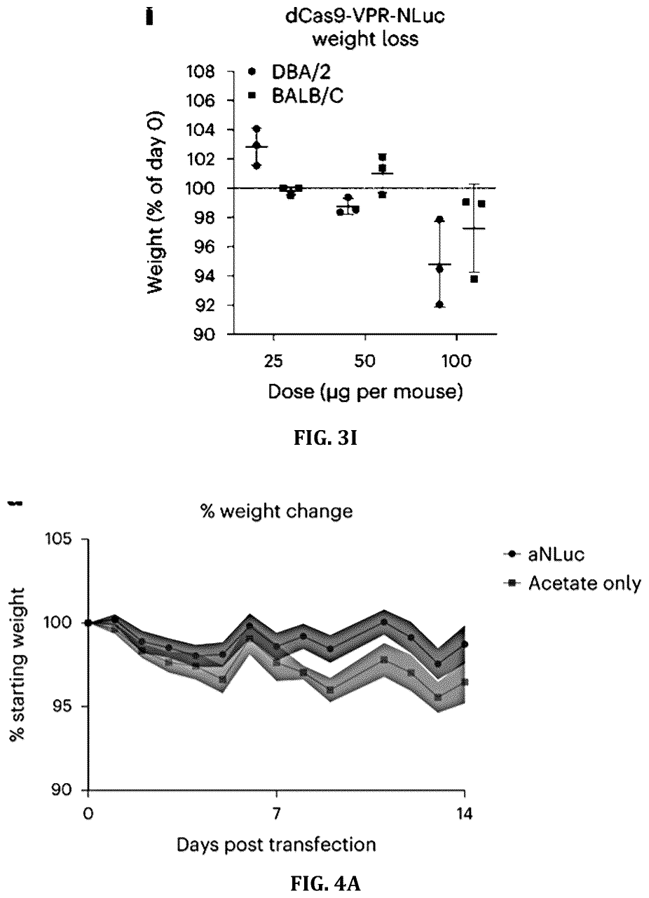

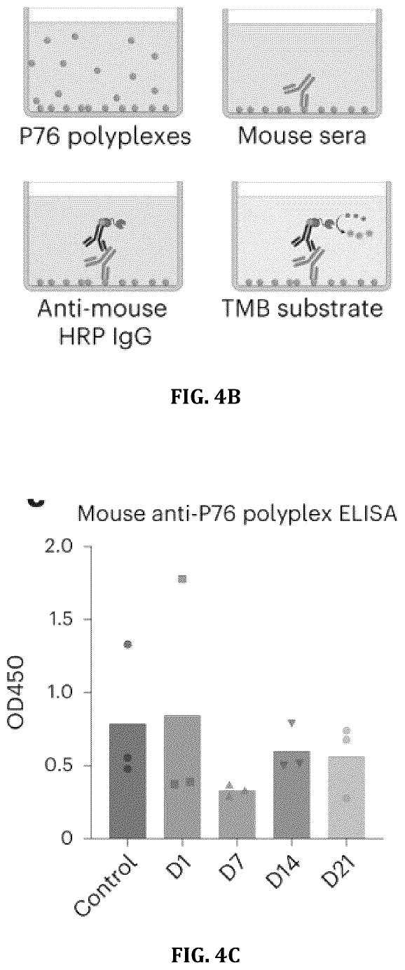

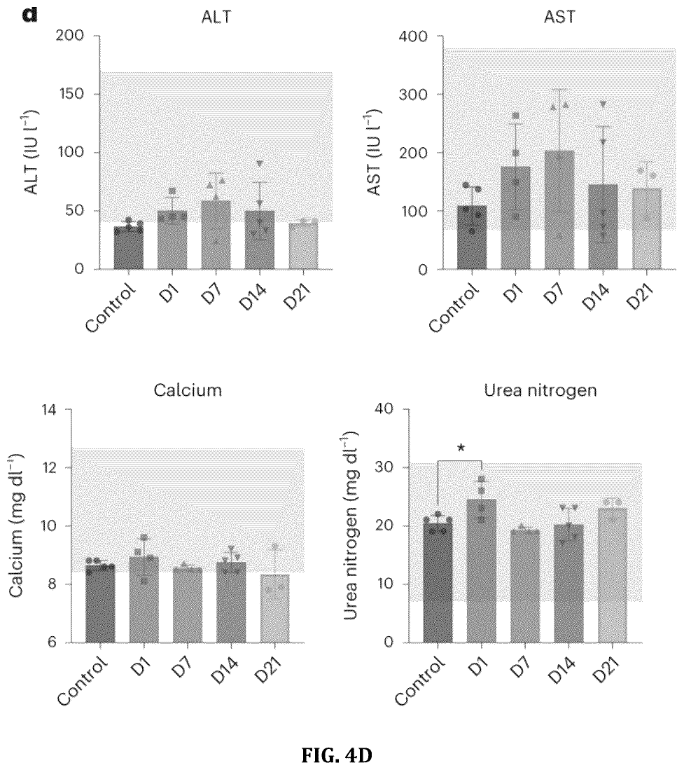

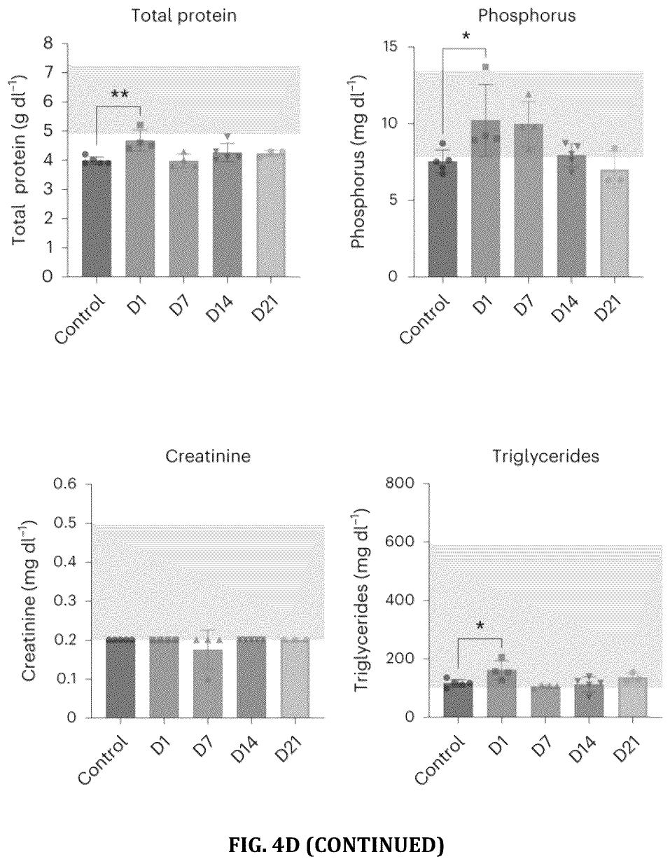

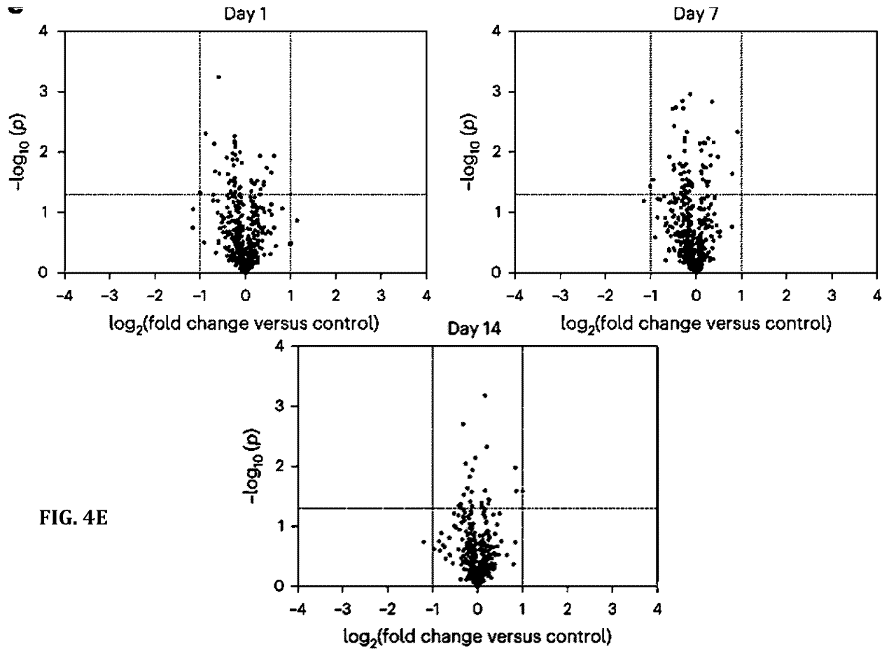

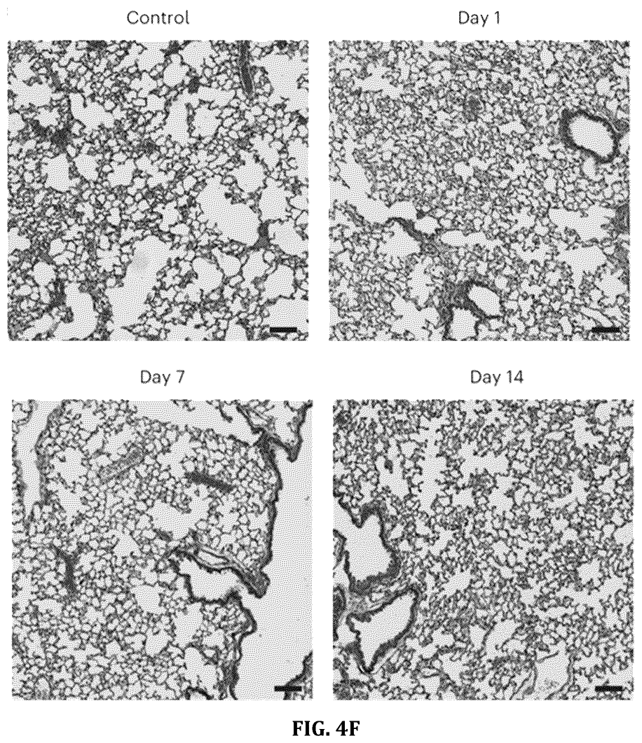

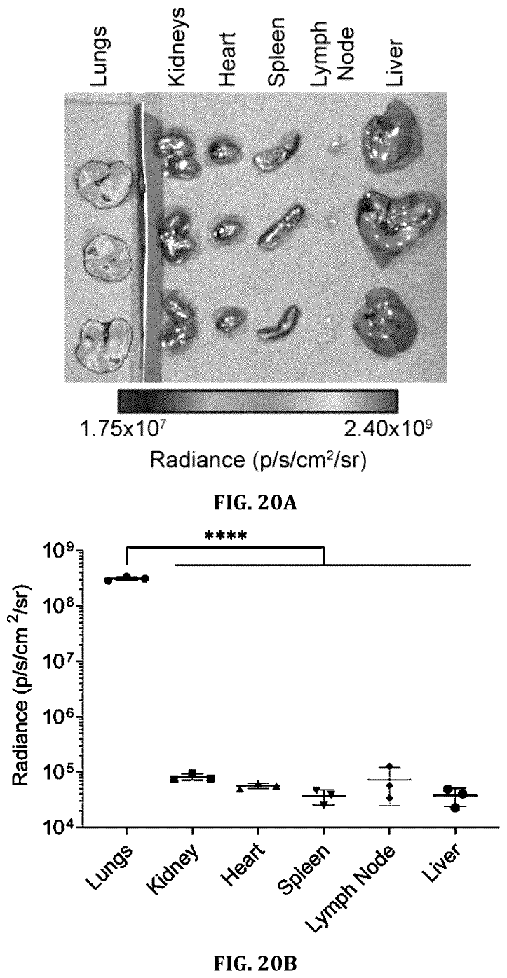

The toxicological effects and biodistribution of nebulized P76 delivery to mice was then examined. 1.25 mg/kg of P76 formulated aNLuc mRNA was delivered and mice were analyzed for: 1) weight changes, 2) serum anti-P76 polyplex antibody levels, 3) complete blood chemistry, 4) lung tissue level differential gene expression, and 5) histopathology over a 21-day period post exposure. Over the first 14 days, no significant difference in weights was observed between the P76-treated and untreated animals (FIG. 4A). Second, potential immunological responses were analzyed by investigating whether treated animals developed anti-P76 polyplex antibodies (FIG. 4B). None of the serum, out to 21 days, had any detectable levels of antibodies over the assay background signal in the control group (FIG. 4C). Next, complete blood chemistry metrics were analyzed (FIG. 4D). While day I levels of total protein, urea-nitrogen, phosphorus, and triglycerides were elevated compared to untreated animals, all of the metrics at all timepoints fell within or below the 95% confidence interval of these measures for normal animals as reported by Charles River. Next, changes in 561 immune marker genes within mouse lungs were investigated at days 1, 7, and 14 days after a single exposure to P76 polyplexes (FIG. 4E). No significantly differentially expressed genes were detected at any time point. This corroborates previous reports of modified mRNA transfection in mice and demonstrates minimal immunological effects of the polymer.²⁵ Next, lung tissue pathology was assessed at 1, 7, and 14 days post P76 polyplex exposure. No animals exhibited any evidence of necrosis, edema, inflammation, or other lesions (FIG. 4F). Finally, no detectable luminescence in the kidneys, heart, spleen, paraaortic lymph node, or liver was observed in the mice which were delivered 5 mg/kg of aNLuc mRNA formulated via P76 (FIGS. 20A-20B). Together, these data indicate neither pathology nor differential gene expression after P76 polyplex delivery via nebulizer, and that functional particle delivery is restricted to the lung.

P76 Delivered mRNA Across Species with Minimal Toxicity

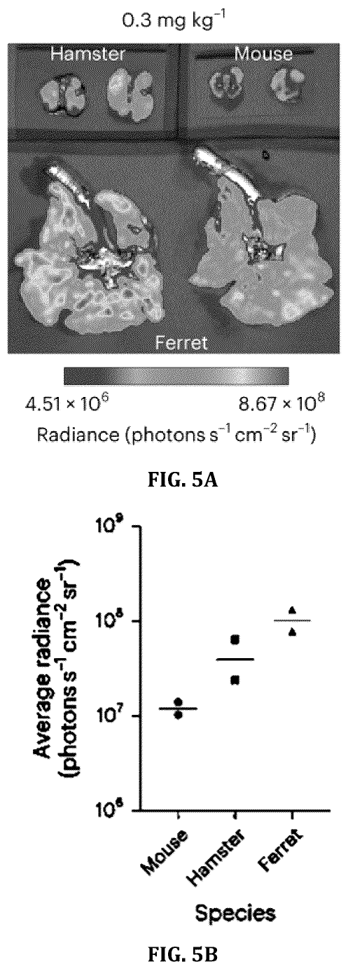

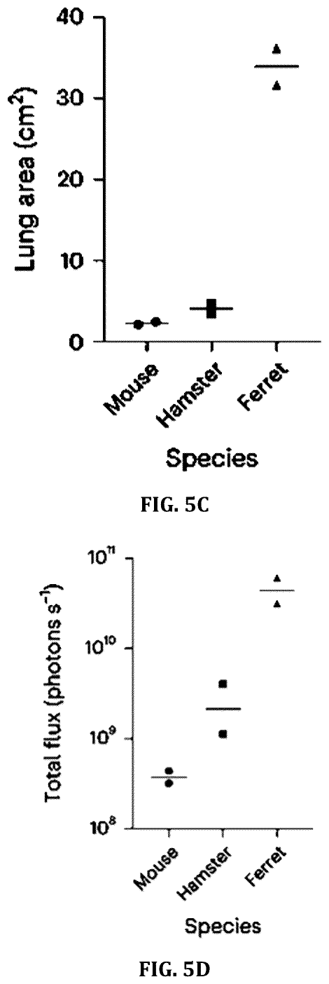

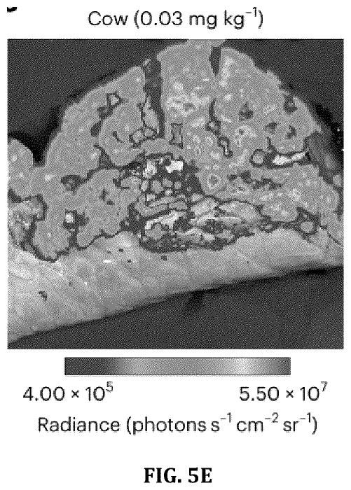

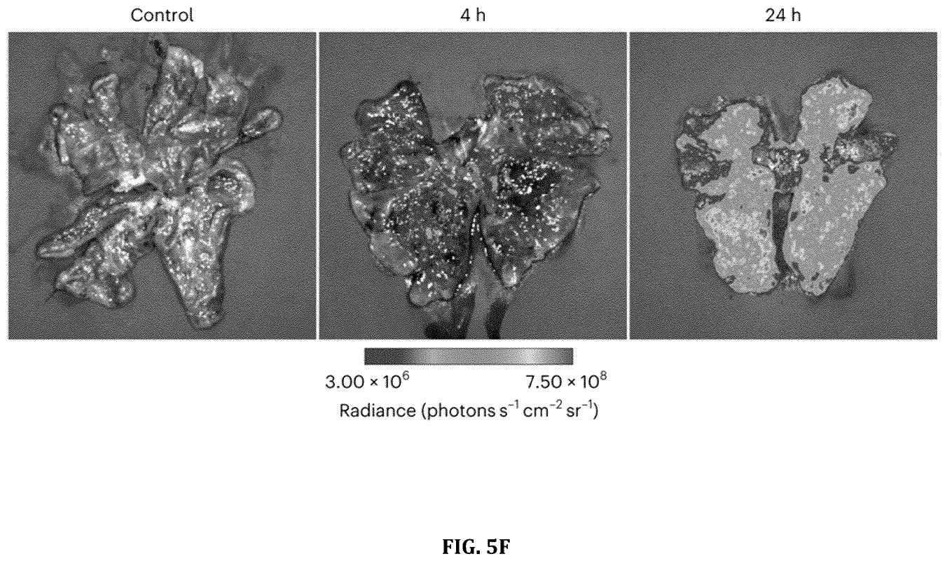

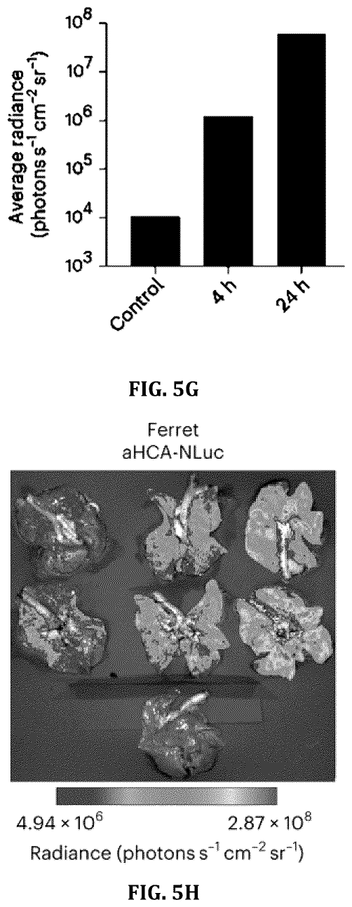

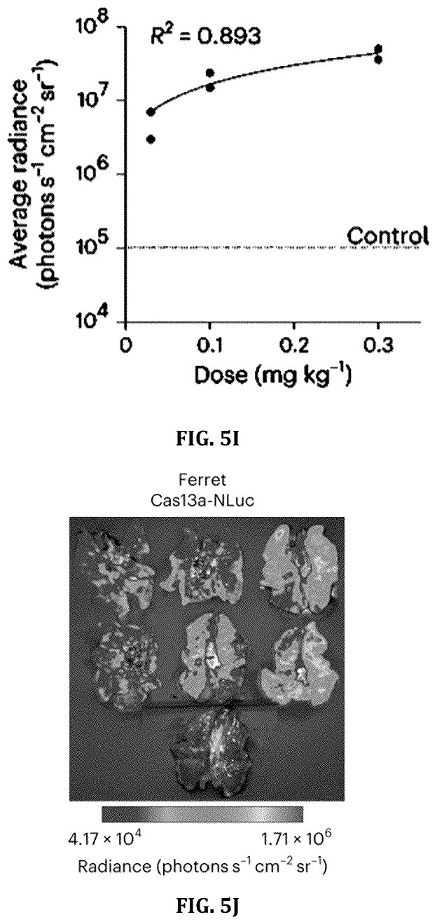

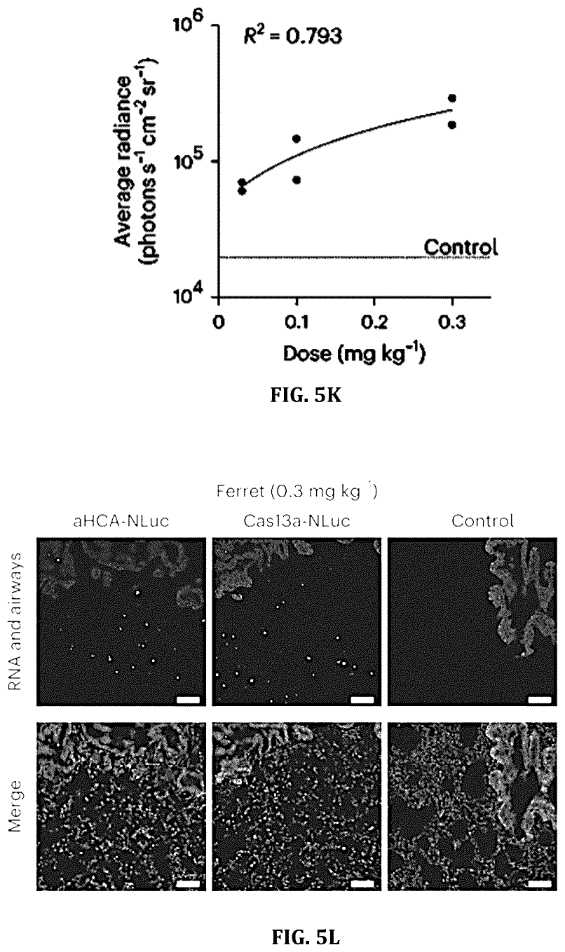

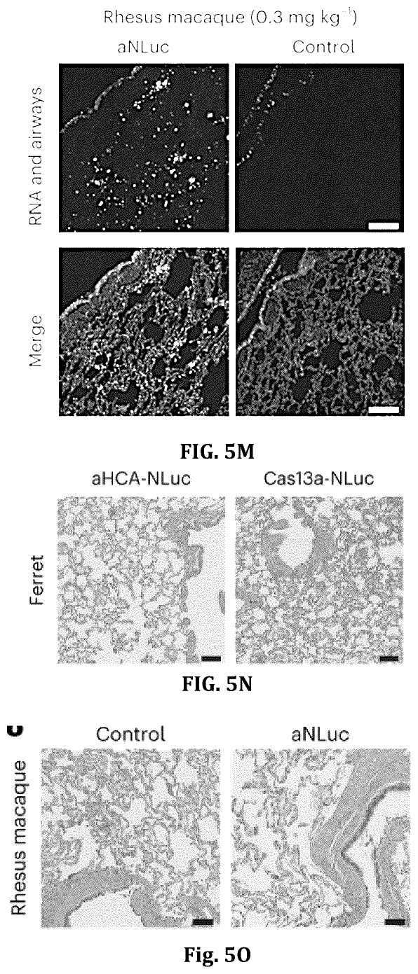

While testing mRNA vehicles in mice is critical for early-stage pharmaceutical testing, preclinical studies require testing the polymers in a variety of species for efficacy and toxicology studies. Delivery of P76 across mice, hamsters, and ferrets was compared at a constant 0.3 mg/kg dose of aNLuc mRNA. Interestingly, the average radiance increased as the animal size increased (FIG. 5A-5D). Specifically, the geometric mean of average radiance was 3.3 and 8.5 times higher in the hamster and ferret lungs, respectively, compared to the mouse lungs, likely due to lower tidal volumes in smaller animals (FIG. 5B-5C). Overall, these data support the use of P76 in mice, hamsters, and ferrets as an efficient vehicle for mRNA therapeutic delivery.

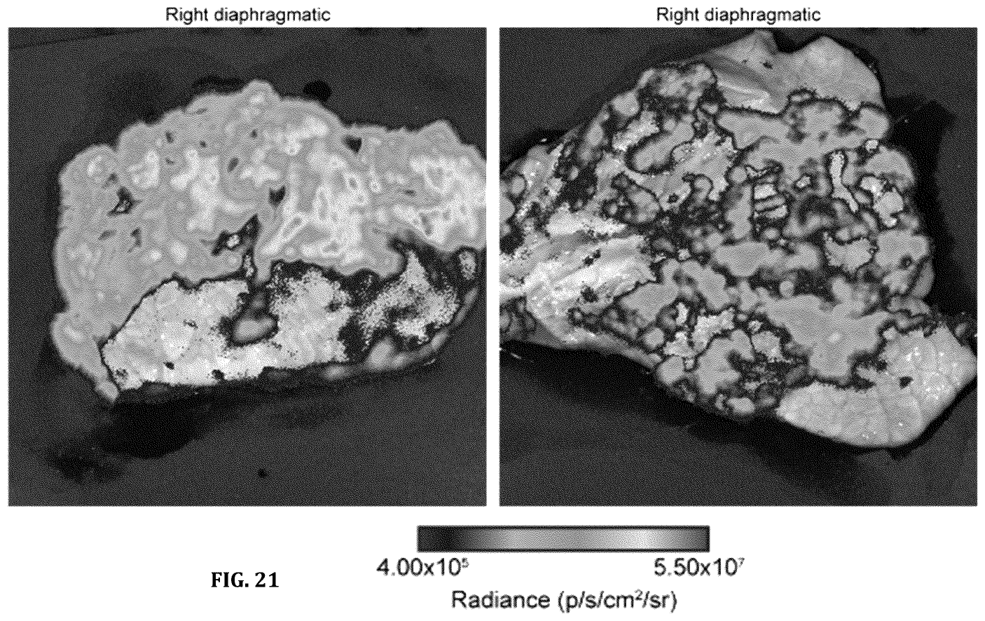

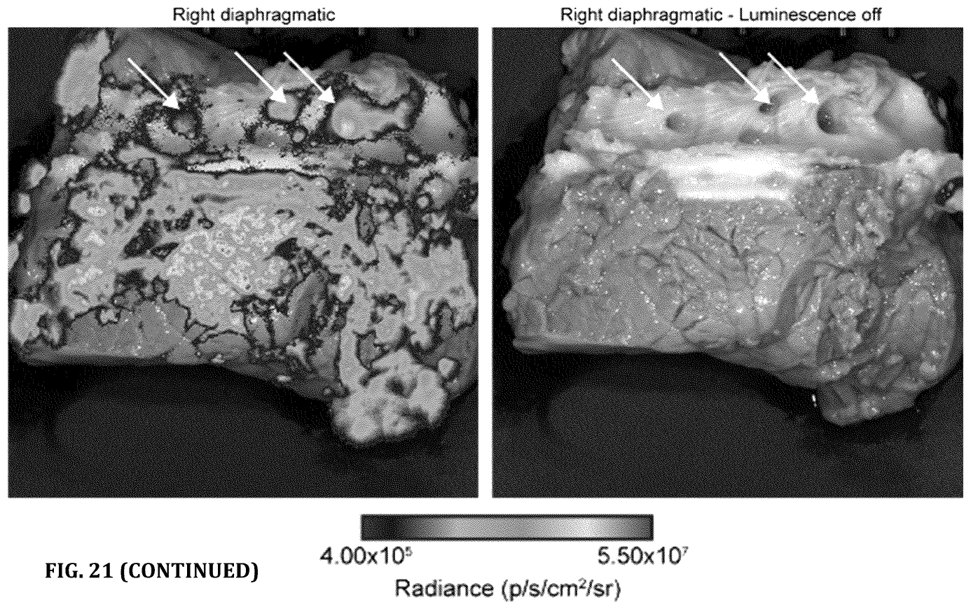

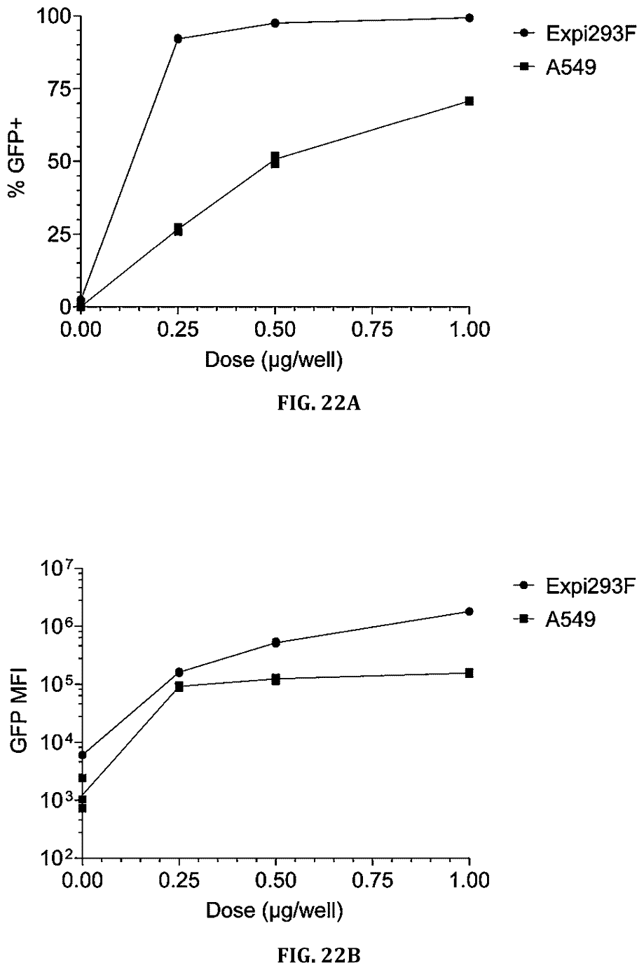

Next, to evaluate delivery in a large animal, delivery of P76 formulated aNLuc mRNA was assessed in an adolescent cow (approximately 113 kg). Cows of this size are an excellent model of human and bovine respiratory syncytial virus (RSV).²⁶ Upon nebulizer delivery of 0.03 mg/kg of aNLuc mRNA, luminescence was observed across the lung tissue, with peak radiances greater than 10⁷ p/s/cm²/sr (FIG. 5E). The 0.03 mg/kg dose was chosen to limit nebulization time since the same Acrogen nebulizer used for the rodents was employed. Finally, delivery via P76 resulted in aNLuc expression even within the larger cartilaginous airways, indicating broad delivery targets in the lung (FIG. 21).

As non-human primates (NHPs) are often predictive of therapeutic and disease responses in humans, a test of nebulized delivery of aNLuc mRNA via P76 was performed in rhesus macaques. Compared to a control animal, P76 resulted in a 116 and 5,610 fold increase in average radiance over control tissue from 4 hours to 24 hours post transfection (FIGS. 5F & 5G). Like the bovine results above, the visceral pleura blocked luminescent substrate delivery to alveolar tissue in some regions. Finally, P76 efficiently delivered mRNA to human cell lines (FIGS. 16A-16B). The high expression of the aNLuc reporter in both bovine and macaque lungs, and in human cells, strongly supports the use of P76 in preclinical research.

Dose response of more relevant mRNA reporter constructs was then evaluated in the ferret to assess expression in larger species. First, doses of 0.03, 0.1, and 0.3 mg/kg of aHCA-NLuc mRNA were tested, finding a significant, linear increase in average radiance with increasing dose (R²=0.893, p=0.0044) (FIGS. 5H & 5I). Similarly, a linear dose response was observed when delivering the same dosages of Cas13a-NLuc mRNA along with a crRNA (R²=0.793, p=0.0173) (FIGS. 5J & 5K). These data provide strong evidence for potent delivery of therapeutic mRNA cargos in the larger ferret model, even at low doses.

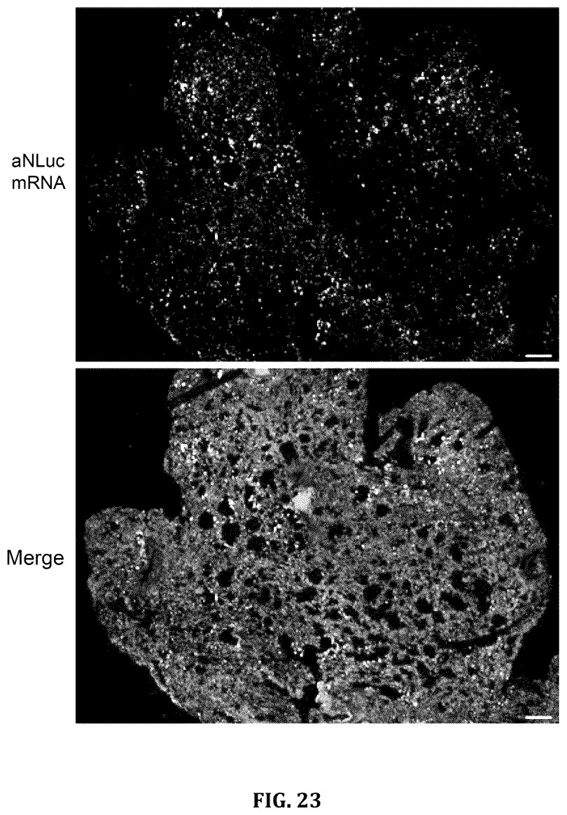

The biodistribution of P76 mediated RNA delivery was assessed at the cellular level in the larger animal models using FISH. In the ferret, well distributed RNA signal was observed for both aHCA-NLuc and Cas13a-NLuc mRNAs, indicating that the length of the mRNA does not significantly impact the distribution of delivery in the lung (FIG. 5L). This trend continued in the larger macaque lung, with aNLuc mRNA appearing well-dispersed throughout the tissue (FIG. 5M and FIG. 23). Given the smaller size and thus, higher number of aNLuc mRNA molecules delivered to the lung (compared to Cas13a-NLuc mRNA), the RNA signal in the macaque appeared much stronger than in the ferrets, despite the equal dosage. In both species, mRNA delivered via P76 primarily targeted cells in the alveolar space rather than airway cells.

Initial toxicity assessment in both the ferret and macaque yielded no significant findings by histological analysis of the treated lungs at 24 hours with a 0.3 mg/kg dose (FIGS. 5N & 5O). Analysis of serum before and after dosing in the macaques revealed minimal increases in serum cytokine levels (Table 5). Last, hematology and blood chemistry analysis revealed only minor changes in most metrics, with all but one remaining within normal levels for macaques (Tables 6, 7). Taken together, these data strongly support the nebulized use of P76 for high mRNA delivery with minimal toxicity in future preclinical applications.

P76 Delivered mRNA and Guide at Therapeutic Levels

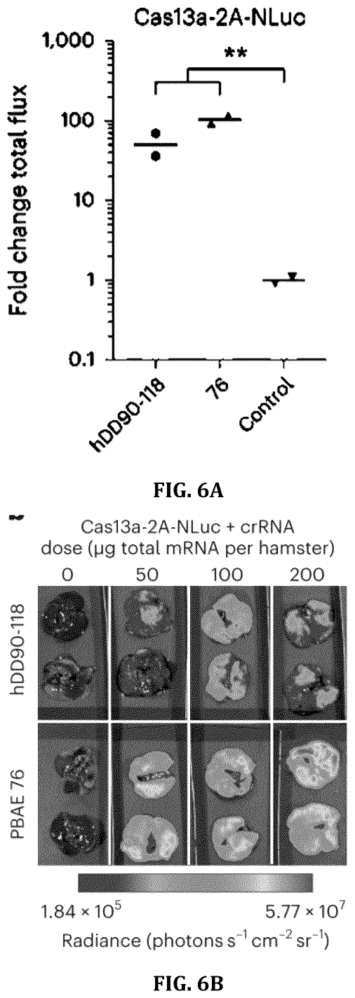

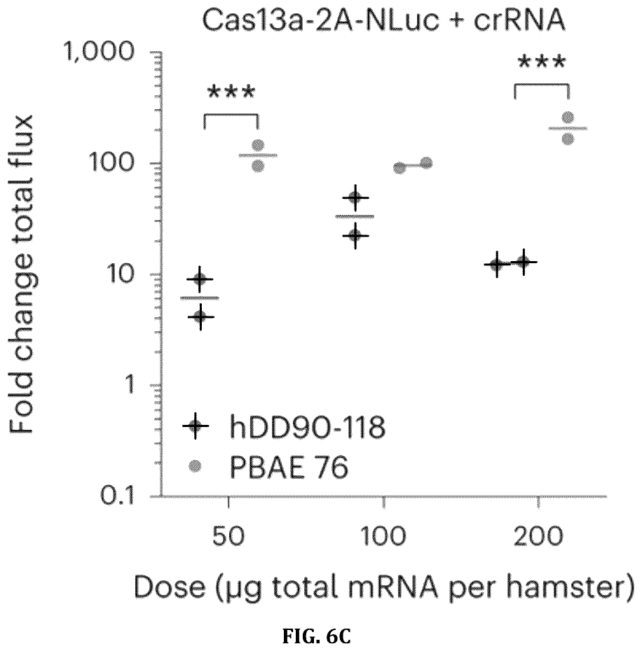

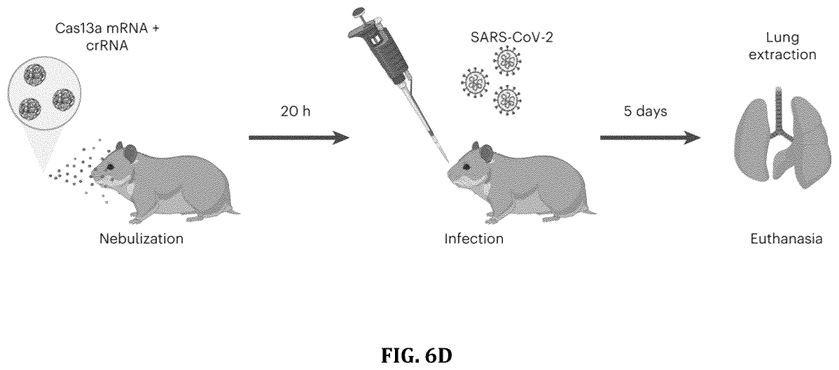

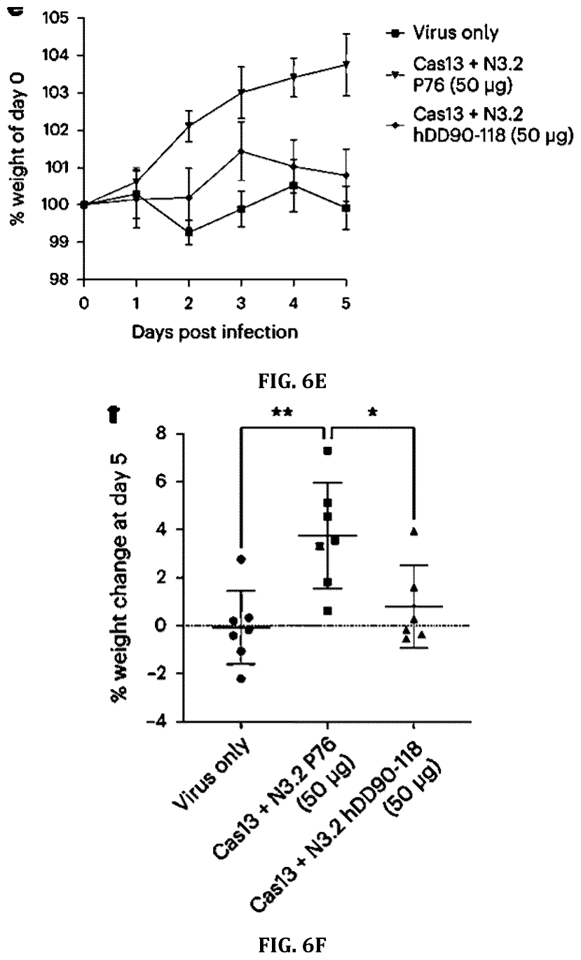

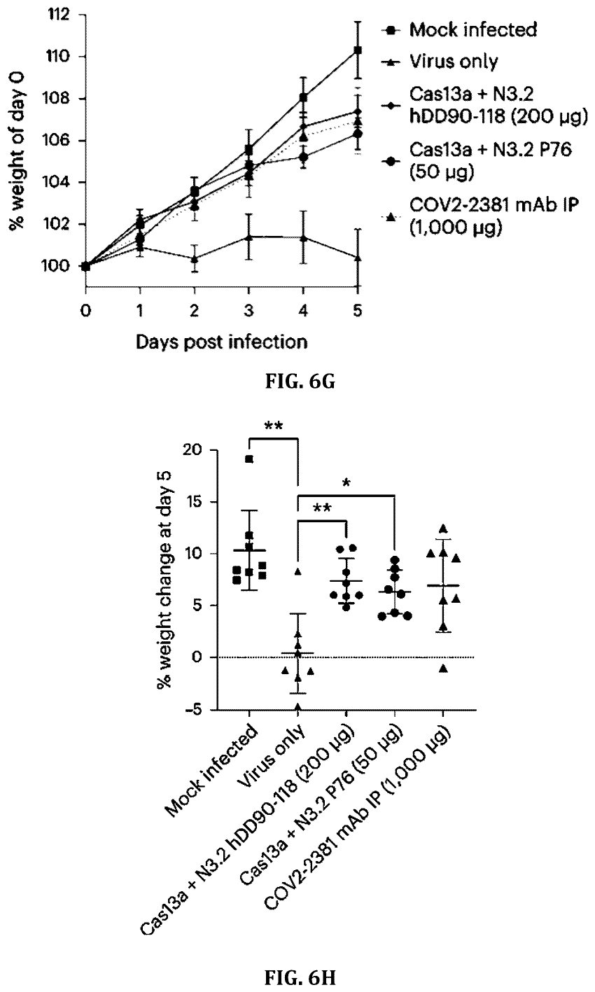

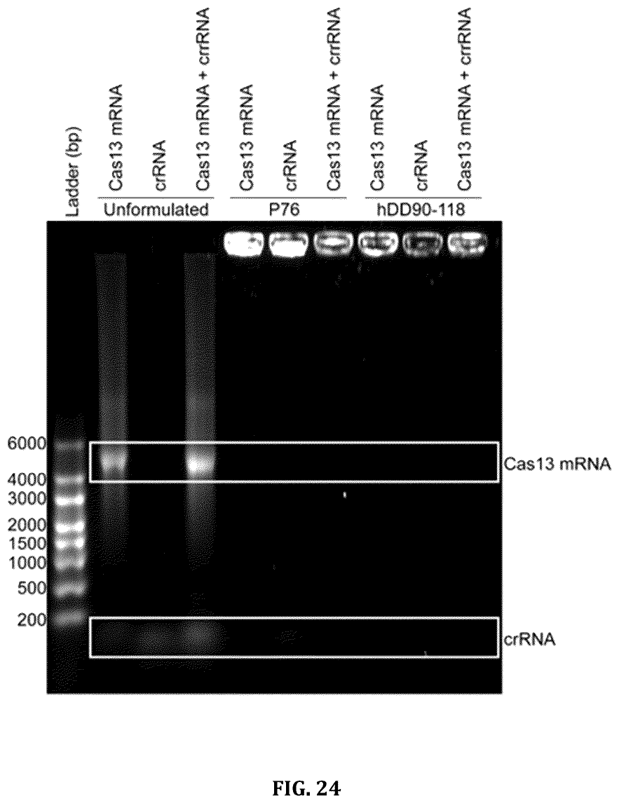

Finally, we compared the therapeutic efficacy of P76 to PBAE hDD90-118. First, the Cas13a-NLuc reporter mRNA was delivered via nebulizer at 1.25 mg/kg to hamsters, finding that P76 delivery resulted in a 2.05-fold increase in signal (FIG. 6B). However, when a crRNA was delivered alongside the Cas13a-NLuc mRNA, P76 formulation produced 19.1, 2.9, and 16.4 times more signal when delivered at 0.31, 0.625, and 1.25 mg/kg, respectively, compared to hDD90-118 (FIGS. 6B & 6C). The increase in Cas 13-NLuc expression was not due to a difference in mRNA and crRNA binding between P76 and hDD90-118 (FIG. 24).

Next, we tested the therapeutic efficacy of P76 formulations in a SARS-COV-2 challenge in the hamster model. Hamsters were treated with LbuCas13a mRNA alongside our previously validated anti-SARS-COV-2 crRNA, N3.2, with a P76 or hDD90-118 formulation and infected intranasally 20 h later with 1,000 PFU of the WA-1 strain of live SARS-COV-2 (FIG. 6D).¹⁰ Hamsters were weighed daily as a measure of general health, and when both the P76 and hDD90-118 formulations were delivered at 0.5 mg/kg, only the P76 formulation prevented differential weight loss due to SARS-COV-2 challenge over 5 days (FIG. 6E). Critically, the animals treated with a 0.5 mg/kg dose of P76 formulated Cas13 mRNA with N3.2 crRNA gained a significant amount of body weight by day 5 compared to both the virus only control group and the 0.5 mg/kg hDD90-118 formulation (FIG. 6F). These data suggest that P76 is significantly more efficient at delivering the Cas13 mRNA and crRNA than hDD90-118, consistent with the luminescence data (FIG. 6C).

To further validate the potency of P76 formulations, we performed the same study with the 0.5 mg/kg P76 formulation and an increased dose of 2 mg/kg of the hDD90-118 formulation. We also compared these mRNA based formulations to one group of hamsters that was treated via intraperitoneal administration with 10 mg/kg of the potent neutralizing monoclonal antibody, COV2-2381, as a gold-standard control.²⁷ One group of hamsters was mock-infected while another group was untreated and infected as a negative and positive control, respectively. We found that the 0.5 mg/kg Cas13a mRNA dose using P76 performed as well as both the 2.0 mg/kg dose using hDD90-118 and the 10 mg/kg dose of COV2-2381, with all treated hamsters gaining 6.88% body weight on average over 5 days (FIG. 6G). Weight change at day 5 was only significantly improved by the hDD90-118 and P76 formulations (FIG. 6H), and there was no significant difference among the treated groups. Interestingly, SARS-COV-2 RNA knockdown in the lung was only significantly reduced by 59.5% and 81.9%, compared to the virus only group, at day 5 in hamsters treated with either P76-formulated Cas13a or COV2-2381, respectively (FIG. 6I), but there was no significant difference between the treated groups.

### Outlook

Efficient pulmonary delivery of mRNA can support a variety of pharmaceutical development programs ranging from antivirals and inhalable vaccines to CF treatment. ^{10, 28, 29} To date, the majority of pulmonary-targeted nucleic acid vehicles have been given systemically. And while the recently reported PBAE, hDD90-118, produced strong expression of mRNA in mice, it was not capable of simultaneously delivering a crRNA without reducing expression of the mRNA-encoded protein.

166 PBAE and PBAE-containing polymers were prepared and screened, identifying P76 as a highly efficient vehicle for mRNA delivery via nebulizer. The P76 PBATE can deliver mRNA to two mice strains, hamsters, ferrets, cows, and rhesus macaques, exhibiting the species-agnostic efficacy of this polymer. Furthermore, delivery in larger species is more efficient, likely through a better matching of their tidal volume and droplet size generated by the nebulizer and the airway and airspace sizes within the lung. Importantly, the incorporation of thiols allowed P76 to efficiently co-deliver short crRNAs with long mRNAs, dramatically increasing the utility of the polymer for any CRISPR-based therapeutic candidate. These properties allowed for a 4× lower dose in a SARS-COV-2 challenge in a hamster model using P76-delivered Cas13a mRNA compared to the previously reported PBAE, with similar efficacy to the gold standard of systemic neutralizing antibody treatment. Also, minimal toxicity of P76 formulations was found in mice, ferrets, and macaques. Together, the PBATE, P76, represents a substantial step forward for polymeric nanoparticle formulations, enabling future inhalable nucleic acid therapeutics.

### EXAMPLES

### Methods

### Polymer Synthesis

Diacrylate and amine monomers were purchased from Sigma-Aldrich, Fisher and TCI America. To synthesize hyperbranched polymers, backbone diacrylate: linear amine: trifunctional amine monomers were mostly reacted at a ratio of 1:0.5:0.2 with some changes for some polymers like 38 and 76 (Table 1). The reactions were performed in anhydrous dimethylformamide at a concentration of 150 mg/mL at 40° C. for 4 h and then 90° C. for 48 h. The resulting mixtures were allowed to cool to 30° C. and a “capping” nucleophile (diamine, aminothiol, or other functionalized amine) was added at 1.5 molar equivalent relative to the excess acrylate and the reaction was stirred for a further 24 h. The polymers were purified by dropwise precipitation into cold anhydrous diethyl ether with 0.1% glacial acetic acid, vortexed and centrifuged at 1,250×g for 2 min to pellet the polymer. The supernatant was discarded and polymer washed twice more with fresh diethyl ether and dried under vacuum for 48 h. Polymers were stored at −20° C. Polymer 76 available upon reasonable request to the corresponding author.

### NMR

Nuclear magnetic resonance (NMR) spectra were obtained on a Bruker DRX-500 or Bruker AV3 HD-700 instrument in CDCl₃ or CD₃OD. All ¹H NMR experiments are reported in δ units, parts per million (ppm), and were measured relative to the signals for residual methanol (3.35 ppm) or chloroform (7.26 ppm).

mRNA Synthesis

Plasmids containing the T7 promoter, 5′ untranslated region (UTR), open reading frame (ORF), and 3′ UTR were used for in vitro transcription (GenScript). The sequences of the glycosylphosphatidylinositol (GPI) anchor and nanoluciferase are reported by Lindsay et. al. ¹⁹ The Leptotrichia buccalis Cas13a sequence is originally obtained from Addgene (p2CT-His-MBP-Lbu_C2C2_WT, plasmid no. 83482), and the NLuc reporter sequence was placed behind a 2A cleavage site following the Cas13a. The VPR sequence was originally obtained from Addgene (SP-dCas9-VPR, plasmid no. 63798) with the same 2A cleavage and NLuc sequence as above. The IgG reporter construct was the human contraceptive antibody (HCA) sequence, originally called H6-3C4, with NLuc fused to the light chain as in Lindsay et. al. ^{19, 30, 31}

mRNA were synthesized as previously described. ¹⁰ Plasmids were linearized with Not-I HF (New England Biolabs) overnight at 37° C. Linearized templates were purified by sodium acetate (Thermo Fisher Scientific) precipitation and rehydrated with nuclease-free water. In vitro transcription was performed overnight at 37° C. using the HiScribe T7 Kit (NEB) following the manufacturer's instructions (N1-methyl-pseudouridine modified). The resulting RNA was treated with DNase I (Aldevron) for 30 min to remove the template, and it was purified using lithium chloride precipitation (Thermo Fisher Scientific). The RNA was heat denatured at 65° C. for 10 min before capping with a Cap-1 structure using guanylyl transferase and 2′-O-methyltransferase (Aldevron). mRNA was then purified by lithium chloride precipitation, treated with alkaline phosphatase (NEB) and purified again. mRNA concentration was measured using a Nanodrop. mRNA stock concentrations were 1-3 mg/mL. Purified mRNA products were analyzed by gel electrophoresis to ensure purity. The crRNA N3.2 guide

(5′ GACCACCCCAAAAAUGAAGGGGACUAAAACGUAUUCAAGGCUCCCUCAGUUGCAACC 3′)

was purchased from Integrated DNA Technologies (IDT).

### Polyplex Formulation

Polyplexes were formulated as described previously.¹⁰ Before delivery to animals, 100 mM of sodium acetate pH 5.0 was used to both solubilize the hyperbranched PBAE and dilute mRNA before mixing. The final concentration of the mRNA was 0.5 mg/mL, and the PBAE was used at a 50× molar ratio to the mRNA. The formulations were incubated at room temperature for 10 min, and the particles were loaded into the nebulizer as described.

### DLS Measurements

Polyplexes were prepared as described for in vivo usage with a final concentration of 0.5 mg/mL nucleic acid. Next, 10 mL of particles were then diluted into 990 mL of 100 mM sodium acetate, pH 5.0 in a sizing cuvette and analyzed using a Malvern Zetasizer Nano ZS. For zeta potential measurements, 0.4 mL of particles were diluted in 4.6 mL H₂O, pH 5.0, and loaded into a Malvern capillary for analysis.

### Animal Studies

Animals at the Georgia Institute of Technology and the University of Georgia were sourced and handled as described previously.¹⁰

Six to eight week-old female BALB/c or DBA/2 mice (Jackson Laboratories) were maintained at the Georgia Institute of Technology in individually ventilated and watered cages kept at negative pressure. BALB/c mice were used in all experiments unless indicated in the figure caption. Mice were kept in rooms on a 12 h light/dark cycle with ambient temperature between 22.8 and 23.9° C. with 30-40% relative humidity. Experiments were only performed during the light phase. Food (Lab Diet 5001) was provided to mice ad libitum. Animals were acclimatized for at least 6 d before the beginning of experiments. Animals were randomly distributed among experimental groups. Researchers were blinded to animal group allocation during data acquisition. Animals were sacrificed by CO₂ asphyxiation. Infected animals were handled and kept under BSL-2 conditions until euthanized.

Hamster delivery optimization experiments were performed at the Georgia Institute of Technology. Four-week-old male LVG Golden Syrian Hamsters (Charles River Laboratories) were maintained in individually ventilated and watered cages kept at negative pressure. Hamsters were kept in rooms on a 12 h light/dark cycle with ambient temperature between 21.1 and 22.8° C. with 35-50% relative humidity. Experiments were only performed during the light phase. Food (Lab Diet 5001) was provided to hamsters ad libitum. Animals were acclimatized for at least 6 d before beginning experiments. Animals were randomly distributed among experimental groups. Researchers were blinded to animal group allocation during data acquisition. Animals were sacrificed by CO₂ asphyxiation.

Male, neutered, and descented fitch ferrets (Marshall BioResources) weighing about 1 kg were maintained at the Georgia Institute of Technology in wire caging with water and food provided ad libitum. Ferrets were kept in a room on a 12 h light/dark cycle with ambient temperature between 22.2 and 22.8° C. with 40-50% relative humidity. Experiments were only performed during the light phase. Ferrets were allowed ˜30 m of physical enrichment time 3 times a week. Animals were acclimatized for at least 6 d before beginning experiments. Animals were randomly distributed among experimental groups. Researchers were blinded to animal group allocation during data acquisition. Animals were euthanized using approximately 2.5 mL of pentobarbital-based veterinary euthanasia drug.

All animals were cared for according to the Georgia Institute of Technology Physiological Research Laboratory policies and under ethical guidance from the university's Institutional Animal Care and Use Committee following National institutes of Health (NIH) guidelines.

Hamster infections were performed at the University of Georgia. Outbred male LVG Golden Syrian hamsters, 3-4 weeks of age, were obtained from Charles River Laboratories. Hamsters were housed inside an animal BSL-3 room in a HEPA-filtered cage/rack system and provided food (Lab Dict 5053) and water ad libitum. Animals were randomly assigned to groups by animal care staff blinded as to study design and treatment. Hamsters were acclimatized before use. Animals were cared for according to the University of Georgia Animal Health Research Center policies and under ethical guidance from the university's Institutional Animal Care and Use Committee (IACUC) following NIH guidelines.

A normal, castrated male Holstein calf 16 weeks of age and weighing 113 kg was enrolled in the study and maintained in a paddock with access to free choice Bermudagrass hay and water and were fed a commercial calf grower ration at 8 pounds daily. Prior to treatment, a physical exam and clinical assessment was performed and was repeated at 6, 12, and 18 h following treatment. At the conclusion of the study the calf was euthanized via IV administration of Beuthanasia D solution (390 mg/mL pentobarbital sodium and 50 mg/mL phenytoin sodium) at a rate of 1 mL per 4.5 kg of body weight. All animal research activities were approved by the Mississippi State University IACUC.

Rhesus macaques (n=3) used in this study were housed in the BSL2+ housing of the New Iberia Research Center and maintained in accordance with the regulations of the Guide for the Care and Use of Laboratory Animal, and the studies were reviewed and approved by the University of Louisiana at Lafayette IACUC. The macaques were fed monkey chow (Purina) supplemented daily with fresh fruit or vegetables with water provided ad libitum. Animals were euthanized using 120 mg/Kg Beuthanasia delivered IV to animals pre-sedated with ketamine.

Nebulized mRNA Polyplex Delivery

Mice and hamster deliveries were performed as described previously.¹⁰ Mice were loaded into a custom-built nose-only exposure system constructed of a clear PVC tee and animal restraints (CODA Small Mouse Holder, Kent Scientific). These were connected using a custom 3D-printed nose cone (3D Printing Tech) made of a flexible TPU material. The nebulizer (Aeroneb, Kent Scientific) was then placed on the upward facing port of the tec. Doses were added dropwise to the nebulizer at a rate of 25 μl per mouse per droplet. After each individual droplet was nebulized, the clear tee was inspected until the vaporized dose had cleared (approximately 15s-45 s per drop). Droplets were added until the desired dose per animal was achieved. After the vapor had cleared after the last droplet, the mice were removed from the restraints.

For delivery of mRNA to hamsters, the exposure system was modified with larger animal restraints (CODA Large Mouse Holder, Kent Scientific) and a larger 3D-printed nose cone to fit the larger restraints. To account for the increase in tidal volume in hamsters compared to mice, doses were added dropwise to the nebulizer at a rate of 62.5 μl per hamster per droplet.

For delivery of mRNA to ferrets, a large ferret restraint (Conduct Science) was used alongside a larger 3D-printed nosecone. Ferrets were dosed one at a time, with 100 μL droplets added to the nebulizer at a time to account for the increase in tidal volume of the larger animals.

For delivery of mRNA to the cow, a nose-only exposure system was created using a modified Erlenmeyer flask with a rubber gasket to seal around the snout. The end of the system was covered in several layers of gauze to collect the exhalant. An Acrogen Solo nebulizer was used with the USB power supply powered by a mobile Li-ion battery. The complete bolus of polyplexes was applied to the nebulizer, and the animal was allowed to breathe normally until the chamber was empty.

For delivery of mRNA to the macaques, a pediatric nebulizer mask was used along with a U-shaped nebulizer adapter tube (Acrogen). Macaques were only handled and dosed under sedation with IM injection of Telazol (4-8 mg/kg). The complete bolus of polyplexes was applied to the nebulizer, and the animal was allowed to breathe normally until the chamber was empty. Animals were held upright to mimic human usage of the facemask setup. Blood was taken via venipuncture prior to delivery and immediately before euthanasia.

### Luminescence Imaging

After euthanasia, whole lungs were collected and rinsed with PBS. For mice, hamsters, and ferrets, lungs were then placed into a solution of Nano-Glo substrate (Promega) diluted 50-fold in PBS. Lungs were incubated for 5 min and then placed onto black paper and imaged with an IVIS Spectrum CT (PerkinElmer). For cow and macaques, lungs were sectioned manually using a scalpel to approximately bisect the lungs or lung lobes. Diluted Nano-Glo substrate was then dropped using a micropipette to thoroughly cover the exposed surface before imaging on the IVIS. Cow tissue was imaged on an IVIS lumina XRMS, Series III. Lung luminescence was then quantified by drawing ROIs around the lungs using Living Image software (version 4.7.4, PerkinElmer). In each experiment, luminescence data is presented with a single radiance range, and white lines are present to separate groups from each other to aid visually.

### SARS-COV-2 Challenge Study

Hamster infections with SARS-COV-2 were performed as described previously.¹⁰ Hamsters were anesthetized by intraperitoneal injection of a mixture of 100 mg/kg of ketamine/5 mg/kg of xylazine. After loss of toe pinch reflex, SARS-COV-2 was administered to each hamster via intranasal route in a total volume of 50 μl. Animals were then administered reversal agent (atipamezole, 0.15 mg/kg) and placed on a heating pad until able to right themselves. Body weights and clinical signs were checked and recorded daily. For sample collection, hamsters were anesthetized as described above and administered pentobarbital (100 mg/kg) via intraperitoneal injection. After exsanguination and pneumothorax, tissues were collected aseptically for analyses.

Whole lungs from individual hamsters were placed in 2 ml of DMEM 1% FBS containing antibiotics/antimycotics (D1) in C tubes and homogenized using a GentleMACS machine at ‘lung 2’ setting (Miltenyi). After centrifugation for 10 m at 1,000×g, supernatant was removed, and remaining homogenates were resuspended in Trizol for RNA extraction. Chloroform-based phase separation and RNA precipitation was then performed followed by two ethanol washes.

### PCR

PCR was performed as described previously.¹⁰ After total RNA quantification by Nanodrop, cDNA was prepared using the High-Capacity cDNA reverse transcription kit (The Applied Biosystems™, Thermo Fisher Scientific). qPCR experiments were performed using the FastAdvanced Master Mix (Thermo Fisher Scientific). The anti-viral activity of Cas13a was measured (n=6) by quantifying the fold change of the viral N gene using the CDC-approved NI primer/probe set (2019-nCOV_N1) and a 18S primer/probe as endogenous control.³² Experiments were performed using a QuantStudio7 Flex thermal cycler (Applied Biosystems).

For quantification of mRNA delivery to mouse lungs, lungs were collected, 5 minutes after P76-mediated Cas13-2A-NLuc or aNLuc mRNA delivery (25 μg/mouse, into 2 mL of Trizol in a Miltenyi C tube. Lungs were homogenized using the RNA_02 setting on a GentleMACS device before being aliquoted and stored at −80° C. Chloroform was added for phase separation for 15 minutes at 16,000 xg. The aqueous phase containing RNA was then mixed with an equal volume of 70% ethanol and purified using an RNeasy plus kit (Qiagen) according the manufacturer's instructions. cDNA was prepared using the RT2 First Strand Kit (Qiagen) and PCR was performed using the same FastAdvanced Master Mix (Thermo Fisher Scientific) as above with the QuantStudio7 Flex thermal cycler (Applied Biosystems).

### CryoEM

Three μL samples collected before and after nebulization were placed on 300-mesh, copper grids (carbon substrate with 1.2 μm holes spaced by 1.3 μm. Quantifoil Micro Tools, Germany). Grids were previously glow-discharged (negative charge) for 15 seconds using a GloQube Plus glow discharge system (Quorum Tech, Laughton, UK). Samples were blotted with filter paper for either 2.5 s or 3 s at room temperature and 100% humidity and plunged into liquid ethane using a Vitrobot Mark IV (ThermoFisher Scientific, Hillsboro, OR). Cryo-EM grids were stored in liquid nitrogen until cryo-EM data collection was done.

Cryo-EM images were acquired using a JEOL JEM1400 TEM (JEOL, Japan) operating at 80 keV. Micrographs were collected at a nominal magnification of 20,000×, on a 2,048 by 2,048 charge-couple device (CCD) camera (UltraScan 1000, Gatan Inc, Pleasanton, CA, USA), yielding a pixel size of 5.1 Angstrom. Overview images were collected at 2,000× (52 Angstrom per pixel). Particle diameters were determined using the measuring tool within Gatan's Digital Micrograph software (Gatan Inc, Pleasanton, CA, USA).

### Histology and Pathology Scoring

H&E lung slides were examined by an ACVP board certified veterinary pathologist. For each animal, all lung lobes were used for analysis and affected microscopic fields were scored semi-quantitatively as Grade 0 (None); Garde 1 (Minimal); Grade 2 (Mild); Grade 3 (Moderate) and Grade 4 (Severe). Scoring was performed based on the following criteria: percent lung affected, type 2 pneumocyte hyperplasia, alveolar septal thickening, inflammatory infiltrates, and severity of broncho-interstitial pneumonia. An average and total lung score per group was calculated by combining scores from each criterion. No significant findings were observed across any of the assayed lungs. Digital images in FIG. 4F of H&E-stained slides were captured by the Cancer Tissue Pathology (CTP) core facility at Emory University and analyzed by a board certified-pathologist. Images in FIGS. 5N & 5O were captured by HistoWiz and analyzed by a pathologist and in the same manner as above.

nCounter Analysis

Tissue was collected into tubes and frozen immediately on dry ice. RNA was extracted using Trizol as described above. RNA concentration and integrity was confirmed by spectrophotometer and BioAnalyzer (Agilent), respectively, before being analyzed on a NanoString nCounter using the mouse immunology panel according to manufacturer's instructions. Fold changes and p-values were calculated using the nSolver software (NanoString).

### Gel Electrophoresis Mobility Retardation Assay

P76 was unformulated or was formulated at a 50:1 mass ratio with Cas13 mRNA, crRNA, or a mix of Cas 13a and crRNA. Unformulated RNA and polyplexes were mixed 1:1 with RNA gel loading dye (ThermoFisher R0641). In all cases, 1.8 μg of Cas13a mRNA or 1.2 μg of crRNA was loaded into a 1% agarose gel and ran at 80 V for 1.5 hours. The gel was visualized using an Axygen Gel Documentation System-BL.

### Statistical Analyses

All experiments are represented as a mean of biologically independent replicates or independent samples as indicated in individual figure captions. Power analyses for group sizes were calculated using G*Power (version 3.1, University of Dusseldorf). Data were analyzed and plotted using GraphPad Prism 9. Statistical analyses were performed between groups using either two-tailed t-tests or ordinary one-way or two-way analysis of variance (ANOVA) as specified in individual figure captions.

### Supplementary Methods

### Macaque Serum Cytokine Measurements and Blood Chemistry and Counts

For blood count, 1 mL of whole blood was collected from each macaque into Etheylenediaminetetraacetic acid (EDTA) tubes and transported under ambient conditions to the University of Louisiana Lafayette-NIRC Clinical Pathology Laboratory. A complete blood count with differential and reticulocyte count was performed using the Beckman Coulter DxH600 hematology analyzer.

For serum chemistry, 2 mL whole blood were collected from each macaque into SST vacutainer tubes and transported under ambient conditions to the UL Lafayette-NIRC Clinical Pathology Laboratory for centrifugation and analysis. Samples were allowed to clot prior to centrifugation for serum harvest. A comprehensive serum chemistry panel was performed at UL Lafayette-NIRC on a Siemens Dimension Clinical Chemistry Analyzer.

Cytokines were measured using Magpix system (Bio-Rad) with the standard reagent (STD) for the non-human primate panel of cytokines (Inflammation 23-Panel, Millipore), and in accordance with the manufacturer's protocol. Briefly, after coating the wells of a 96-well plate with Magpix assay buffer, 25 ml plasma sample, 25 ml of one of six 4-fold serial dilutions STD, 25 ml Magpix assay buffer and 25 ml Magpix magnetic beads were added to each well. Magpix scrum matrix was used instead of plasma in background controls. After overnight incubation with constant agitation for 17 hours at 4° C., the plate was washed four times with Magpix washing buffer and Magpix magnetic plate device. Next, 25 ml Magpix detection antibodies were added to each well, and the plate was incubated with agitation at room temperature for 1 h, Then, 25 ml of streptavidin-phycocrythrin solution was added to each well, and the plate incubated with agitation at room temperature for 30 min. After further washing steps, the magnetic beads were resuspended in 150 ml washing buffer, and the plate was analyzed using Magpix plate reader. Cytokine concentrations were calculated using xPONENT 4.2 software and the application of nonlinear regression for standard curves, in which lower and upper limits of quantification were determined.

### In Situ Hybridization

At 4 hours post-delivery, animals were sacrificed, perfused with 1×PBS, and lungs were removed and incubated in 4% paraformaldehyde overnight at 4° C. Paraffin embedded 5 mm sections were prepared by HistoWiz. Delivered mRNA and endogenous mRNA was visualized using RNAscope Multiplex Fluorescent Reagent Kit v2 (Advanced Cell Diagnostics 323136) according to manufacturer's instructions. A custom probe set was designed against the synthetic aNLuc mRNA sequence (ACD 879571), aHCA-NLuc light chain mRNA sequence (ACD 1058321-C1), and Cas13a-NLuc mRNA sequence (ACD 1058341-C1). To distinguish lung airways, probes for secretoglobin (Scgb1a1, mouse: ACD 420351-C3, ferret: ACD 300040, macaque: ACD 1058211-C2) and Forkhead Box JI (Foxj1, ferret: ACD 1058181-C3, and macaque: ACD 1058221-C3) were used. Lung tissue was scanned by the Emory Winship Cancer Tissue and Pathology core on a Perkin Elmer Vectra Polaris slide scanner. Images were processed and quantified using Imaris 9.7.2 software (Bitplane) and figure images were produced using QuPath v0.3.0. For high resolution images, slides were imaged using a PerkinElmer UltraVIEW spinning disk confocal with a Hamamatsu Flash 4 sCMOS camera and Zeiss 63×NA 1.40 plan-apochromat objective lens mounted on a Zeiss Axiovert 200 M body. Images were acquired as a Z-stack using an ASI PZ-2150 stage with 0.2 mm slices using Volocity version 6.3.1 (Perkin Software).

To quantify RNA positive regions in mouse lungs using Imaris (Bitplanc), a region of interest (ROI) was drawn by hand around the lung regions, excluding the trachea, heart and other ancillary tissues. RNA was found via absolute intensity within this ROI and created as a surface. Mean and area measurements were then plotted.

### Molecular Dynamics Simulations

All-atom Molecular Dynamics (MD) was applied to investigate the interactions between the polymer and the mRNA. Only a monomer of the polymer being studied was built using the Molefacture plug-in of Visual Molecular Dynamics (VMD).³² From the crystal structure of RNase (7DIC), the nine-base-pair mRNA strand was selected and used to represent the mRNA utilized in experiments.³³ One monomer was placed 50 Å away from the nine-base-pair mRNA from the crystal structure. The experimental molar ratios of the monomer to the number of base pairs of RNA is reflected in the systems, with one monomer to 11 basepairs. The mRNA and monomer were solvated in a TIP3P water box with 0.10M NaCl.³⁴ The dimensions of the water box were 87 Å×87 Å×87 Å.

The mRNA and monomer were restrained for 1 ns with the water unrestrained to minimize and equilibrate the system. Following this equilibration, the system was released and run for 1000 ns per replica, with three replicas total per system. In total, 6 us of simulation was run between all systems and replicas. Production simulations were run on Georgia Tech's Hive cluster, using NAMD^{34, 35} while initial equilibration were run using NAMD2.³⁶ Each monomer was parameterized using CGenFF v1.0.0 and force field v3.0.1^{37, 38} and the CHARMM36³⁹ force-field was used for water, ions, and RNA. All simulations were run at 310 K with constant pressure at 1 atm, enforced using Langevin dynamics with the damping coefficient at 1/ps and the Langevin piston method⁴⁰, and periodic boundary conditions. A timestep of 2 fs was utilized for the first 1 ns restraint, followed by a timestep of 4 fs with Hydrogen Mass Repartition (HMR)⁴¹ for the full production runs. Van der waals interactions were cutoff at 12 Å with a switching function beginning at 11 Å. Long-range electrostatic interactions were calculated with the particle-mesh Ewald method+2, using a grid spacing of less than 1 Å.

Hydrogen bond analysis between specific selections of the monomer and the RNA was quantified using the Hydrogen Bond plug-in of VMD. Hydrogen bonds were defined using cut-offs 3.5 Å (D-A distance) and 30° (A-D-H angle). Contact frequency was calculated between the RNA and a selection of the monomer over the entire 1000 ns trajectories. The range of interactions was defined as within 4 Å.

### Mouse Serum Blood Chemistry and Anti-P76 Polyplex ELISA

Mice were exsanguinated under isoflurane anesthesia. Blood was collected via the jugular vein into gel serum separator tubes (BD Microtainer SST tubes). On average, 800 mL of whole blood was collected. Serum was separated by centrifugation at 1000×g for 10 minutes. A 50 mL aliquot of serum was reserved for ELISA analysis, and the remainder was used for serum chemistry measurements. Serum chemistry was analyzed by Antech. Samples with insufficient serum volume or excess hemolysis were excluded from analysis. After euthanasia, mice lungs were extracted and either frozen for Nanostring analysis or placed in formalin for H&E processing.

For anti-P76 polyplex ELISA, high-binding 96-well plates (Corning) were coated overnight at 4° C. using 50 mL of a 1:500 dilution of aNLuc mRNA polyplexes diluted in sodium acetate buffer. Plate was washed 3× in 1×PBS with 0.05% Tween-20 (PBST) using a plate washer (Biotek). Wells were blocked using 1% BSA for 1 hr at RT with shaking before another set of 3× washes. Serum samples, diluted 1:100 in 1% BSA, were incubated for 2 hr at RT with shaking before a set of 4× washes. Donkey anti-mouse HRP (Jackson Immunoresearch, 715-035-150, 1:1000) was then added and the plate was incubated for another 2 hr at RT with shaking. After 4× washes, signal was developed using 1-step Ultra TMB (Thermo Fisher Scientific). Reaction was stopped with 2M sulfuric acid (Sigma) and absorbance was read at 450 nm using a Synergy H1 plate reader (Biotek).

### Transfection and Analysis of Human Cell Lines

Expi293F (ThermoFisher A14527) and A549 (ATCC CCL-185) cells were maintained in either Expi293 Expression Medium or Dulbecco's Modified Eagle Medium (DMEM) with 10% FBS (Hyclone) and 1% penicillin-streptomycin (Corning), respectively. GFP mRNA was diluted to 1 mg/mL and P76 was diluted to 50 mg/mL in 25 mM sodium acetate buffer (pH 5.0) and mixed at a volume ratio of 1:1. After a 10 min incubation at room temperature, the mRNA formulation was diluted to 10, 5, 2.5, and 0 mg/mL in Opti-MEM (ThermoFisher). 100 mL of each formulation was treated to Expi293F (1.8×10⁶ cells/well in 900 mL of Expi293 Expression Medium) and A549 (1.8×10⁵ ccells/well in 900 μL of DMEM) cells in 24-well plates. Expi293F cells were incubated at 37° C., 8% CO₂ with an orbital shaker at 230 rpm for 24 h. A549 cells were incubated at 37° C., 5% CO₂ for 24 h and trypsinized at 37° C. for 5 min. The cells were then collected and stained using Live/Dead Fixable Violet Dead Cell Stain Kit (ThermoFisher) at room temperature for 30 min. The stained cells (1×10⁵ cells of Expi293F and 3×10⁴ cells of A549) were analyzed using a Cytek Aurora flow cytometer, and GFP expression was calculated with FlowJo (v10.7.0).

### Chemical Analysis and Performance of the Polymers

All of the polymers, but especially P76 and hDD90-118, exhibited highly positive surface zeta potentials, indicating enhanced binding of the negatively charged mRNA cargo, prevention of aggregation in solution, and promotion of lung delivery (FIG. 15A).⁴³⁻⁴⁵ Next, it was determined whether the rapid shaking of the mesh nebulizer significantly altered the size or shape of polyplexes using the polymers. All lead particles, as well as hDD90-118, had diameters of 180 nm or less when measured by dynamic light scattering (DLS), with only P38 having a significantly smaller diameter after nebulization (FIG. 15B). Conversely, particles formulated with either hDD90-118 or P147 significantly increased in size upon nebulization, with hDD90-118 particles increasing from 140.2 to 148.3 nm in diameter, and P147 particles increasing from 161.1 to 180.3 nm in diameter. Furthermore, when we examined the particle shapes of P76 using cryo-transmission electron microscopy (cryoEM), we observed largely spherical particles, with no significant change in particle size or shape after nebulization (FIG. 15C, FIG. 16). Together, these results demonstrate that P76 potently delivers mRNA to the lungs of mice, due in part to a highly positive surface charge, with minimal structural changes from the nebulization process.

To understand how the P76 PBATE binds RNA and influences nucleic acid nanoparticle delivery, molecular dynamic simulations were performed on a P76 subunit alongside a short single-stranded RNA oligomer. As expected, hydrogen bonding between the oxygen atoms in the P76 backbone and the bases of the mRNA were observed, as well as π-π interactions between the bisphenol A and the RNA bases (FIG. 15D). Interestingly, the sulfur groups interacted with the RNA backbone, likely through hydrophobic interactions, making a more stable bond between the polymer and RNA cargo. aNLuc mRNA was formulated and delivered at 10, 25, 50, 75, and 100:1 (P76: mRNA), and a significant reduction in expression was measured when used at the 10:1 mass ratio compared to all other tested ratios, with an average decrease of 99.5% in luminescence (FIG. 17). No significant differences were measured between any of the other tested ratios. Thus, we elected to keep the 50:1 mass ratio that was used previously with hDD90-118.⁴⁶

### P76 Delivery to Human Cells.

To confirm that P76 delivers mRNA to human cells, we assessed the transfection efficiency and expression of green fluorescent protein (GFP) mRNA in both A549 (human lung epithelial) and Expi293F (human kidney epithelial) cell lines. Cells were transfected with 250, 500, and 1000 ng per well of GFP RNA formulated with P76 or left untreated. Using flow cytometry, a peak of 70.8% and 99.3% transfected A549 or Expi293F cells was observed, respectively, at the 1000 ng per well dose (FIG. 21). Similarly, the mean fluorescence intensity (MFI) was highest at the 1000 ng per well dose for both cell types (FIG. 21). These results support the use of P76 in future human studies.

P76 Efficiently Binds Cas13 mRNA and crRNA

To determine whether or not the increase in Cas13-NLuc expression (FIG. 6C) was due to a difference in binding capacity for the different lengths of the RNA cargos, binding of both hDD90-118 and P76 to Cas13 mRNA and crRNA was assessed, separately and when mixed, via a gel retardation assay (FIG. 23). Both hDD90-118 and P76 significantly retarded the migration of the mRNA and crRNA, regardless of if the RNAs were mixed or separate. This suggests that both hDD90-118 and P76 bind both lengths of RNA with similar efficiency, and that there is another mechanism responsible for the increased protein expression when using P76, likely either improved endosomal escape or polymeric release of RNA into the cytosol.

### Tables

TABLE 1
Polymer formulations and ratios used in screening. All
chiral structures not composed of sterols, sugars,
or amino acids were made from racemic components.

| | | | | | End- |
|---|---|---|---|---|---|
| Polymer ID | Backbone | | Branching | Linker | capping |
| A | 1 | | 43 | 20 | 52 |
| B | 4 | | 21 | 20 | 52 |
| C | 18 | | 21 | 20 | 57 |
| H | 18 | | 21 | 20 | 52 |
| 1 | 4 | (0.5) | 21 | 20 | 52 |
| | 5 | (0.5) | | | |
| 2 | 13 | (0.7) | 21 | 20 | 52 |
| | 5 | (0.3) | | | |
| 3 | 13 | (0.5) | 21 | 20 | 52 |
| | 5 | (0.5) | | | |
| 4 | 13 | | 21 | 20 | 52 |
| 5 | 5 | | 32 | 44 | 53 |
| 6 | 5 | | 41 | 22 | 53 |
| 7 | 9 | | 21 | 20 | 52 |
| 8 | 1 | | 43 | 20 | 52 |
| 9 | 3 | | 21 | 20 | 52 |
| 10 | 1 | | 47 | 20 | 21 |
| 11 | 3 | (0.5) | 32 | 20 | 54 |
| | 5 | (0.5) | | | |
| 12 | 3 | (0.95) | 21 | 20 | 52 |
| | 10 | (0.05) | | | |
| 13 | 3 | | 21 | 20 | 52 |
| 14 | 3 | (0.5) | 32 | 20 | 54 |
| | 5 | (0.5) | | | |
| 15 | 3 | (0.95) | 21 | 20 | 52 |
| | 10 | (0.05) | | | |
| 16 | 3 | | 41 | 22 | 52 |
| 17 | 17 | | 32 | 44 | 52 |
| 18 | 17 | | 46 | 44 | 61 |
| 19 | 1 | (0.7) | 30 | 20 | 54 |
| | 18 | (0.3) | | | |
| 20 | 1 | (0.7) | 21 | 20 | 54 |
| | 17 | (0.3) | | | |
| 21 | 9 | (0.5) | 21 | 20 | 54 |
| | 5 | (0.5) | | | |
| 22 | 9 | (0.5) | 21 | 20 | 52 |
| | 10 | (0.5) | | | |
| 23 | 1 | (0.7) | 30 | 20 | 54 |
| | 10 | (0.3) | | | |
| 24 | 1 | (0.5) | 21 | 20 | 57 |
| | 10 | (0.5) | | | |
| 25 | 13 | (0.7) | 42 | 20 | 52 |
| | 5 | (0.3) | | | |
| 26 | 13 | (0.7) | 30 | 20 | 52 |
| | 5 | (0.3) | | | |
| 27 | 13 | (0.7) | 21 | 20 | 54 |
| | 5 | (0.3) | | | |
| 28 | 13 | (0.7) | 21 | 28 | 52 |
| | 5 | (0.3) | | | |
| 29 | 13 | (0.7) | 42 | 20 | 52 |
| | 10 | (0.3) | | | |
| 30 | 13 | (0.7) | 21 | 20 | 52 |
| | 10 | (0.3) | | | |
| 31 | 13 | (0.7) | 32 | 20 | 52 |
| | 10 | (0.3) | | | |
| 32 | 19 | (0.7) | 21 | 20 | 52 |
| | 10 | (0.3) | | | |
| 33 | 1 | | 21 | 20 | 54 |
| 34 | 1 | | 42 | 20 | 52 |
| 35 | 1 | | 21 | 23 | 52 |
| 36 | 1 | | 40 | 20 | 52 |
| 37 | 1 | | 39 | 20 | 52 |
| 38 | 1 | | — | 20 | 52 |
| 39 | 13 | (0.7) | 21 | 23 | 52 |
| | 5 | (0.3) | | | |
| 40 | 13 | (0.7) | 40 | 20 | 52 |
| | 5 | (0.3) | | | |
| 41 | 13 | (0.7) | 39 | 20 | 52 |
| | 5 | (0.3) | | | |
| 46 | 13 | | Amino acid mixture | | 52 |
| 47 | 3 | | 21 | 20 | 52 |
| 48 | 3 | (0.5) | 21 | 20 | 54 |
| | 5 | (0.5) | | | |
| 49 | 3 | | 42 | 23 | 52 |
| 50 | 3 | | 39 | 28 | 52 |
| 51 | 2 | | 21 | 20 | 52 |
| 52 | 2 | | 34 | 28 | 56 |
| 53 | 2 | (0.5) | 21 | 20 | 52 |
| | 10 | (0.5) | | | |
| 54 | 2 | | 34 | 27 | 60 |
| 55 | 7 | | 21 | 20 | 52 |
| 56 | 7 | | 34 | 27 | 56 |
| 57 | 7 | | 21 | 20 | 52 |
| | 10 | | | | |
| 58 | 7 | | 38 | 27 | 58 |
| 59 | 6 | | 21 | 20 | 52 |
| 60 | 6 | | 35 | 28 | 56 |
| 61 | 6 | (0.8) | 21 | 20 | 52 |
| | 10 | (0.2) | | | |
| 62 | 6 | | 39 | 23 | 28 |
| 63 | 13 | (0.7) | Amino acid mixture | | 56 |
| | 5 | (0.3) | | | |
| 64 | 9 | (0.3) | Amino acid mixture | | 56 |
| | 5 | (0.7) | | | |
| 65 | 9 | (0.3) | Amino acid mixture | 56 | 52 |
| | 10 | (0.7) | | | |
| 66* | 1 | | 21 | 20 | 52 |
| 67 | 13 | (0.7) | Amino acid mixture | 52 | 52 |
| | 5 | (0.3) | | | |
| 68 | 1 | | Amino acid mixture | 52 | 52 |
| 69 | 1 | (0.5) | 21 | 20 | 52 |
| | 10 | (0.5) | | | |
| 70 | 1 | (0.5) | — | 20 | 52 |
| | 10 | (0.5) | | | |
| 71 | 13 | (0.7) | 20 | 52 | 52 |
| | 5 | (0.3) | | | |
| 72 | 1 | (0.95) | 21 | 20 | 52 |
| | 11 | (0.05) | | | |
| 73 | 1 | (0.95) | 21 | 20 | 52 |
| | 15 | (0.05) | | | |
| 74 | 1 | (0.95) | 21 | 20 | 52 |
| | 17 | (0.05) | | | |
| 75 | 1 | | 20 | 52 | 52 |
| 76 | 1 | | 31 (0.1) | 20 | 52 |
| | | | 21 (0.4) | | |
| 77 | 1 | | 21 | 26 (0.1) | 52 |
| | | | | 20 (0.1) | |
| 78 | 1 | | 21 | 29 (0.1) | 52 |
| | | | | 20 (0.1) | |
| 79 | 3 | | — | 29 | 52 |
| 80 | 13 | (0.7) | — | 54 | 56 |
| | 5 | (0.3) | | | |
| 81 | 13 | (0.7) | — | 23 | 54 |
| | 5 | (0.3) | | | |
| 82 | 1 | | — | 28 | 52 |
| 83 | 3 | | — | 20 | 52 |
| 84 | 3 | | — | 26 | 56 |
| 85 | 3 | | — | 29 | 59 |
| 86 | 1 | | — | 24 | 52 |
| 87 | 1 | | 36 | 24 | 52 |
| 88 | 1 | | — | 29 | 60 |
| 89 | 8 | | — | 20 | 54 |
| 90 | 8 | | 36 | 24 | 52 |
| 91 | 8 | | — | 29 | 59 |
| 92 | 1 | (0.7) | — | 20 | 54 |
| | 10 | (0.3) | | | |
| 93 | 1 | | — | 29 | 56 |
| 94 | 1 | | 36 | 29 | 52 |
| 95 | 1 | | — | 25 | 24 |
| 96 | 1 | | — | 29 | 24 |
| 97 | 1 | | — | 20 | 54 |
| 98^{#} | 1 | | 21 | 20 | 54 |
| 99 | 5 | | — | 45 | 54 |
| 100 | 5 | | — | 46 | 54 |
| 101 | 5 | | — | 48 | 54 |
| 102 | 5 | | — | 49 | 54 |
| 103 | 10 | (0.5) | — | 45 | 54 |
| | 1 | (0.5) | | | |
| 104 | 10 | (0.7) | — | 45 | 54 |
| | 1 | (0.3) | | | |
| 105 | 10 | (0.5) | — | 45 | 54 |
| | 1 | (0.7) | | | |
| 106 | 8 | | — | 20 | 54 |
| 107 | 1 | | — | 20 | 54 |
| 108 | 10 | (0.5) | 46 (0.2) | | 54 |
| | 1 | (0.5) | 24 (0.2) | | |
| | | | 36 (0.2) | | |
| 109 | 110 | (0.5) | 46 (0.2) | | 54 |
| | 1 | (0.5) | 24 (0.2) | | |
| | | | 36 (0.2) | | |
| 110 | 5 | | — | 46 (0.8) | — |
| 111 | 1 | | — | 29 | 54 |
| 112 | 1 | | 20 | 55 | 112 |
| 113 | 10 | (0.5) | — | 48 | 54 |
| | 1 | (0.5) | | | |
| 114 | 10 | (0.7) | — | 48 | 54 |
| | 1 | (0.3) | | | |
| 115 | 10 | (0.3) | — | 48 | 54 |
| | 1 | (0.7) | | | |
| 116 | 1 | | — | 36 | 52 |
| 117 | 14 | (0.7) | — | 24 | 52 |
| | 5 | (0.3) | | | |
| 118 | 14 | (0.7) | 36 | 24 | 52 |
| | 5 | (0.3) | | | |
| 119 | 14 | (0.7) | — | 20 | 54 |
| | 5 | (0.3) | | | |
| 120 | 14 | (0.7) | — | 37 | 52 |
| | 5 | (0.3) | | | |
| 121 | 14 | (0.7) | 36 | 24 | 52 |
| | 5 | (0.3) | | | |
| 122 | 14 | (0.7) | — | 24 | 52 |
| | 5 | (0.3) | | | |
| 123 | 14 | (0.7) | 36 | 24 | 52 |
| | 5 | (0.3) | | | |
| 124 | 14 | (0.7) | 20 | 54 | 52 |
| | 5 | (0.3) | | | |
| 126 | 14 | (0.7) | — | 37 | 52 |
| | 5 | (0.3) | | | |
| 127 | 10 | (0.5) | 45 (0.2) | | 54 |
| | 14 | (0.5) | 36 (0.2) | | |
| | | | 24 (0.2) | | |
| 128 | 14 | (0.5) | 46 (0.2) | | 54 |
| | 10 | (0.5) | 36 (0.2) | | |
| | | | 24 (0.2) | | |
| 129 | 5 | | 45 | 29 | — |
| 130 | 5 | | 45 | 28 | — |
| 131 | 5 | | 46 | 29 | — |
| 132 | 5 | | 46 | 28 | — |
| 133 | 5 | | 48 | 29 | — |
| 134 | 5 | | 48 | 28 | — |
| 135 | 5 | | 50 | 29 | — |
| 136 | 5 | | 50 | 54 | — |
| 137 | 5 | | 36 | 48 | 54 |
| 138 | 5 | | — | 46 | 54 |
| 139 | 5 | (0.7) | — | 24 | 52 |
| | 8 | (0.3) | | | |
| 140 | 5 | (0.7) | 36 | 55 | 52 |
| | 8 | (0.3) | | | |
| 142 | 16^{b} | | 21 | 52 | 52 |
| 143 | 1 | (0.95) | 21 | 52 | 52 |
| | 16^{b} | (0.05) | | | |
| 144 | 1 | (0.98) | 20 | 50 | 52 |
| | 16^{b} | (0.02) | | | |
| 145 | 16^{b} | (0.5) | 21 | 45 | 52 |
| | 1 | (0.5) | | | |
| 146 | 16^{a} | (0.2) | 21 | 46 | 52 |
| | 1 | (0.8) | | | |
| 147 | 16^{b} | (0.5) | 21 | 47 | 52 |
| | 1 | (0.5) | | | |
| 148 | 16^{b} | | 46 | — | — |
| 149 | 16^{c} | | 30 | 46 | 52 |
| 150 | 16^{c} | | 48 | 46 | 52 |
| 151 | 1 | (0.2) | 21 | 48 | 54 |
| | 16^{b} | (0.8) | | | |
| 152 | 1 | (0.2) | 21 | 48 | 54 |
| | 16^{a} | (0.8) | | | |
| 153 | 16^{a} | | 31 (0.4) | 48 | — |
| 154 | 16^{b} | | 31 (0.4) | 48 | — |
| 155 | 1 | (0.5) | 31 | 48 | 54 |
| | 16^{b} | (0.5) | | | |
| 156 | 1 | (0.5) | 31 | 48 | 54 |
| | 16^{a} | (0.5) | | | |
| 157 | 16^{b} | | 48 | — | 54 |
| 158 | 16^{a} | | 48 | — | 54 |
| 159 | 1 | (0.5) | 31 | 20 | 54 |
| | 16^{b} | (0.5) | | | |
| 160 | 1 | (0.5) | 31 | 20 | 54 |
| | 16^{a} | (0.5) | | | |

| 161 | 1 | 31 | 20 | 54 |
|---|---|---|---|---|
| 162† | 1 | 21 | 20 | 52 |
| 163 | 1 | 31 | — | 28 |
| 164 | 1 | 31 | 20 | 28 |
| 165 | 1 | 36 (0.4) | 29 | 28 |
| | | 31 (0.1) | | |
| 166 | 1 | — | 29 | 52 |

*Modified workup.
^{#}DMPU as solvent.
†¹³C DMF as solvent.
^{a}Mn 575.
^{b}Mn 700.
^{c}Mn 1000.
The monomer labels present in Table 1 below correspond to FIGS. 7B-7E.

TABLE 2
Table 2. P-value list of all screening assays in FIG. 8
and FIG. 1G. One-way ANOVA with Dunnett's multiple comparisons
on log-transform of each group compared to the control
included in each screening assay. p < 0.05 was considered
significant and given an asterisk based on p-value. p >
0.05 was considered not significant (ns).

| | | |
|---|---|---|
| Dunnett's multiple | | Adjusted |
| comparisons test | Summary | P Value |

Screen 1

| | | |
|---|---|---|
| Control vs. 1 | ns | 0.4592 |
| Control vs. 2 | **** | <0.0001 |
| Control vs. 3 | **** | <0.0001 |
| Control vs. 4 | ** | 0.0098 |
| Control vs. 5 | ns | 0.9997 |
| Control vs. 6 | ns | 0.9999 |
| Control vs. hDD90-118 | **** | <0.0001 |

Screen 2

| | | |
|---|---|---|
| Control vs. 9 | **** | <0.0001 |
| Control vs. 10 | **** | <0.0001 |
| Control vs. 11 | ns | 0.4587 |
| Control vs. 12 | * | 0.0208 |
| Control vs. hDD90-118 | **** | <0.0001 |

Screen 3

| | | |
|---|---|---|
| Control vs. 17 | ns | 0.8536 |
| Control vs. 18 | ns | 0.9225 |
| Control vs. 19 | **** | <0.0001 |
| Control vs. 20 | ns | 0.9997 |
| Control vs. 21 | ns | 0.9708 |
| Control vs. 22 | **** | <0.0001 |
| Control vs. 23 | ns | 0.9926 |
| Control vs. 23* | ns | 0.9977 |
| Control vs. hDD90-118 | **** | <0.0001 |

Screen 4

| | | |
|---|---|---|
| Control vs. 24 | **** | <0.0001 |
| Control vs. 25 | ns | 0.3249 |
| Control vs. 26 | ns | >0.9999 |
| Control vs. 27 | ns | 0.2806 |
| Control vs. 28 | ns | 0.9555 |
| Control vs. 29 | ns | >0.9999 |
| Control vs. 30 | * | 0.0257 |
| Control vs. 31 | ns | 0.1179 |
| Control vs. 32 | ns | 0.0985 |
| Control vs. 4A | ns | >0.9999 |
| Control vs. hDD90-118 | **** | <0.0001 |

Screen 5

| | | |
|---|---|---|
| Control vs. 39 | ns | 0.9425 |
| Control vs. 40 | ns | 0.0964 |
| Control vs. 41 | * | 0.0108 |
| Control vs. 46 | ns | 0.1131 |
| Control vs. 47 | ns | 0.4201 |
| Control vs. 48 | ns | 0.991 |
| Control vs. 33 | ns | 0.595 |
| Control vs. 34 | *** | 0.0006 |
| Control vs. 35 | ns | 0.6613 |
| Control vs. 36 | ns | 0.464 |
| Control vs. 37 | ns | 0.0609 |
| Control vs. 38 | **** | <0.0001 |
| Control vs. 49 | ** | 0.0019 |
| Control vs. 50 | ns | 0.1082 |
| Control vs. hDD90-118 | **** | <0.0001 |

Screen 6

| | | |
|---|---|---|
| Control vs. hDD90-118 | **** | <0.0001 |
| Control vs. 51 | **** | <0.0001 |
| Control vs. 52 | ns | 0.6843 |
| Control vs. 55 | **** | <0.0001 |
| Control vs. 62 | ns | 0.9846 |
| Control vs. 63 | ns | 0.6052 |
| Control vs. 69 | **** | <0.0001 |
| Control vs. 70 | **** | <0.0001 |
| Control vs. 71 | **** | <0.0001 |
| Control vs. 72 | **** | <0.0001 |
| Control vs. 73 | **** | <0.0001 |
| Control vs. 74 | **** | <0.0001 |
| Control vs. 75 | **** | <0.0001 |
| Control vs. 76 | **** | <0.0001 |
| Control vs. 77 | **** | <0.0001 |
| Control vs. 81 | ns | 0.2408 |
| Control vs. 82 | **** | <0.0001 |

Screen 7

| | | |
|---|---|---|
| Control vs. 78 | **** | <0.0001 |
| Control vs. 79 | ns | 0.2972 |
| Control vs. 83 | ** | 0.0021 |
| Control vs. 86 | **** | <0.0001 |
| Control vs. 87 | **** | <0.0001 |
| Control vs. 90 | ns | 0.4248 |
| Control vs. 92 | *** | 0.0002 |
| Control vs. 93 | ns | 0.9842 |
| Control vs. 89 | ns | 0.9996 |
| Control vs. 94 | **** | <0.0001 |
| Control vs. 97 | **** | <0.0001 |
| Control vs. 98 | ** | 0.0013 |
| Control vs. hDD90-118 | **** | <0.0001 |

Screen 8

| | | |
|---|---|---|
| Control vs. 107 | **** | <0.0001 |
| Control vs. 108 | **** | <0.0001 |
| Control vs. 109 | **** | <0.0001 |
| Control vs. 110* | *** | 0.0002 |
| Control vs. 111 | ns | >0.9999 |
| Control vs. 113 | *** | 0.0002 |
| Control vs. 99 | ns | 0.9998 |
| Control vs. 100 | ns | 0.9921 |
| Control vs. 101 | ns | 0.9918 |
| Control vs. 103 | ns | 0.2015 |
| Control vs. 104 | ns | 0.999 |
| Control vs. 105 | ** | 0.0029 |
| Control vs. 114 | * | 0.023 |
| Control vs. 115 | **** | <0.0001 |
| Control vs. 116 | **** | <0.0001 |
| Control vs. hDD90-118 | **** | <0.0001 |

Screen 9

| | | |
|---|---|---|
| Control vs. 117 | * | 0.044 |
| Control vs. 119 | ns | 0.386 |
| Control vs. 126 | ns | 0.8829 |
| Control vs. 131 | ns | 0.7665 |
| Control vs. 120 | ns | 0.0515 |
| Control vs. 125 | * | 0.0273 |
| Control vs. 122 | ns | 0.5164 |
| Control vs. 121 | * | 0.0114 |
| Control vs. 124 | ns | 0.6256 |
| Control vs. 107 | **** | <0.0001 |
| Control vs. 118 | ns | 0.2122 |
| Control vs. hDD90-118 | **** | <0.0001 |

Screen 10

| | | |
|---|---|---|
| Control vs. 120 | ** | 0.003 |
| Control vs. 121 | **** | <0.0001 |
| Control vs. 118 | ns | 0.2043 |
| Control vs. 117 | * | 0.0113 |
| Control vs. 119 | ns | 0.4358 |
| Control vs. 122 | ns | 0.9693 |
| Control vs. 137 | ns | 0.9899 |
| Control vs. 125 | ns | 0.8744 |
| Control vs. 138 | ns | 0.8011 |
| Control vs. 140 | * | 0.011 |
| Control vs. 139 | ns | 0.9996 |
| Control vs. hDD90-118 | **** | <0.0001 |

Screen 11

| | | |
|---|---|---|
| Control vs. A | ns | 0.9914 |
| Control vs. B | * | 0.037 |
| Control vs. C | **** | <0.0001 |
| Control vs. H | *** | 0.0009 |

Screen 12

| | | |
|---|---|---|
| Control vs. 147 | **** | <0.0001 |
| Control vs. 149 | ns | 0.9971 |
| Control vs. 146 | ** | 0.0047 |
| Control vs. 116 | **** | <0.0001 |
| Control vs. 150 | ns | 0.9996 |
| Control vs. 145 | *** | 0.0003 |
| Control vs. 142 | ns | 0.9994 |
| Control vs. 143 | **** | <0.0001 |
| Control vs. hDD90-118 | **** | <0.0001 |

TABLE 3
Table 3. P-value list of statistical comparisons made in FIGS. 1E, 2B, 2D,
2E, 2F, 3B, 3E, 3H, 4D, 6A, 6C, 6F, 6H, and 6I. The specific statistical
test used in each figure part is listed in the table along with the p-value
for each comparison. p < 0.05 was considered significant and given
an asterisk based on p-value. p > 0.05 was considered not significant (ns).

| | | |
|---|---|---|
| Tukey's multiple | | Adjusted |
| comparisons test | Summary | P Value |

FIG. 1E

| | | |
|---|---|---|
| Control vs. 3.3 | ** | 0.0055 |
| Control vs. 6.6 | ** | 0.0024 |
| Control vs. 9.9 | ** | 0.0015 |
| 3.3 vs. 6.6 | ns | 0.3713 |
| 3.3 vs. 9.9 | ns | 0.1227 |
| 6.6 vs. 9.9 | ns | 0.6761 |

FIG. 2B

| | | |
|---|---|---|
| 38 vs. 76 | **** | <0.0001 |
| 38 vs. 94 | ns | 0.5973 |
| 38 vs. 116 | ns | 0.9739 |
| 38 vs. 147 | ** | 0.0019 |
| 38 vs. Control | **** | <0.0001 |
| 76 vs. 94 | **** | <0.0001 |
| 76 vs. 116 | *** | 0.0002 |
| 76 vs. 147 | **** | <0.0001 |
| 76 vs. Control | **** | <0.0001 |
| 94 vs. 116 | ns | 0.2437 |
| 94 vs. 147 | * | 0.0286 |
| 94 vs. Control | **** | <0.0001 |
| 116 vs. 147 | *** | 0.0006 |
| 116 vs. Control | **** | <0.0001 |
| 147 vs. Control | **** | <0.0001 |

FIG. 2D

| | | |
|---|---|---|
| hDD90-118 vs. 38 | ns | 0.9921 |
| hDD90-118 vs. 76 | ns | 0.8074 |
| hDD90-118 vs. 94 | * | 0.0385 |
| hDD90-118 vs. 116 | ** | 0.0033 |
| hDD90-118 vs. 147 | ns | 0.0579 |
| 38 vs. 76 | ns | 0.5397 |
| 38 vs. 94 | ns | 0.068 |
| 38 vs. 116 | ** | 0.0051 |
| 38 vs. 147 | ns | 0.104 |
| 76 vs. 94 | * | 0.0117 |
| 76 vs. 116 | ** | 0.0014 |
| 76 vs. 147 | * | 0.0167 |
| 94 vs. 116 | ns | 0.1931 |
| 94 vs. 147 | ns | 0.9983 |
| 116 vs. 147 | ns | 0.1253 |

| | | |
|---|---|---|
| Šidák's multiple | | |
| comparisons test | | Adjusted |
| DBA/2 - BALB/C | Summary | P Value |

FIG. 3B

| | | |
|---|---|---|
| 0 | ns | >0.9999 |
| 25 | ns | 0.2362 |
| 50 | ns | 0.1999 |
| 100 | * | 0.0284 |

FIG. 3E

| | | |
|---|---|---|
| 0 | ns | >0.9999 |
| 25 | ns | 0.9981 |
| 50 | ns | 0.1199 |
| 100 | ns | 0.6081 |

FIG. 3H

| | | |
|---|---|---|
| 0 | ns | >0.9999 |
| 25 | ns | 0.2531 |
| 50 | ns | 0.1545 |
| 100 | * | 0.0165 |

FIG. 4D

| One-way ANOVA | Summary | P Value |
|---|---|---|
| AST | ns | 0.4664 |
| ALT | ns | 0.3612 |
| Creatinine | ns | 0.3994 |
| Calcium | ns | 0.5102 |

| | | |
|---|---|---|
| Dunnett's multiple | | |
| comparisons test | Summary | Adjusted P value |

Urea-nitrogen

| | | |
|---|---|---|
| Control vs. D1 | * | 0.0394 |
| Control vs. D7 | ns | 0.8509 |
| Control vs. D14 | ns | 0.9997 |
| Control vs. D21 | ns | 0.3286 |

Total protein

| | | |
|---|---|---|
| Control vs. D1 | ** | 0.0036 |
| Control vs. D7 | ns | >0.9999 |
| Control vs. D14 | ns | 0.2998 |
| Control vs. D21 | ns | 0.5044 |

Phosphorus

| | | |
|---|---|---|
| Control vs. D1 | * | 0.0357 |
| Control vs. D7 | ns | 0.0607 |
| Control vs. D14 | ns | 0.9752 |
| Control vs. D21 | ns | 0.9624 |

Triglycerides

| | | |
|---|---|---|
| Control vs. D1 | * | 0.022 |
| Control vs. D7 | ns | 0.8695 |
| Control vs. D14 | ns | 0.9958 |
| Control vs. D21 | ns | 0.5372 |

FIG. 6A

| Tukey's multiple | | Adjusted |
|---|---|---|
| comparisons test | Summary | P Value |
| hDD90-118 vs. 76 | ns | 0.1729 |
| hDD90-118 vs. Control | ** | 0.0019 |
| 76 vs. Control | ** | 0.0012 |

FIG. 6C

| Šidák's multiple | | |
|---|---|---|
| comparisons test | | Adjusted |
| hDD90-118 - PBAE 76 | Summary | P Value |
| 50 | *** | 0.0006 |
| 100 | ns | 0.0837 |
| 200 | *** | 0.0008 |

FIG. 6F

| | | Adjusted |
|---|---|---|
| Holm-Šidák's multiple comparisons test | Summary | P Value |
| Virus only vs. Cas13 + N3.2 P76 50 μg | ** | 0.0036 |
| Virus only vs. Cas13 + N3.2 hDD90-118 50 μg | ns | 0.4053 |
| Cas13 + N3.2 P76 50 μg vs. Cas13 + N3.2 hDD90-118 50 μg | * | 0.0207 |

FIG. 6H

| | | Adjusted |
|---|---|---|
| Dunnett's T3 multiple comparisons test | Summary | P Value |
| Mock Infected vs. Virus Only | ** | 0.0014 |
| Mock Infected vs. Cas13a + N3.2 hDD90-118 200 μg | ns | 0.5143 |
| Mock Infected vs. Cas13a + N3.2 P76 50 μg | ns | 0.1951 |
| Mock Infected vs. COV2-2381 mAb IP 1000 μg | ns | 0.6707 |
| Virus Only vs. Cas13a + N3.2 hDD90-118 200 μg | ** | 0.0082 |
| Virus Only vs. Cas13a + N3.2 P76 50 μg | * | 0.0243 |
| Virus Only vs. COV2-2381 mAb IP 1000 μg | ns | 0.0637 |
| Cas13a + N3.2 hDD90-118 200 μg vs. Cas13a + N3.2 P76 | ns | 0.9687 |
| 50 μg | | |
| Cas13a + N3.2 hDD90-118 200 μg vs. COV2-2381 mAb IP | ns | >0.9999 |
| 1000 μg | | |
| Cas13a + N3.2 P76 50 μg vs. COV2-2381 mAb IP 1000 μg | ns | >0.9999 |

FIG. 6I

| | | Adjusted |
|---|---|---|
| Tukey's multiple comparisons test | Summary | P Value |
| COV2-2381 mAb IP 1000 μg vs. Virus only | *** | 0.0009 |
| COV2-2381 mAb IP 1000 μg vs. Cas13a + N3.2 P76 50 μg | ns | 0.5013 |
| COV2-2381 mAb IP 1000 μg vs. Cas13a + N3.2 hDD90-118 | ns | 0.1332 |
| 200 μg | | |
| Virus only vs. Cas13a + N3.2 P76 50 μg | * | 0.013 |
| Virus only vs. Cas13a + N3.2 hDD90-118 200 μg | ns | 0.067 |
| Cas13a + N3.2 P76 50 μg vs. Cas13a + N3.2 hDD90-118 | ns | 0.8005 |
| 200 μg | | |

TABLE 4
Table 4. P-value list of statistical comparisons made in FIGS. 15A,
15B, 16, and 18B. The specific statistical test used in each figure
part is listed in the table along with the p-value for each comparison.
p < 0.05 was considered significant and given an asterisk based
on p-value. p > 0.05 was considered not significant (ns).
FIG. 15A

| Tukey's multiple | | Adjusted |
|---|---|---|
| comparisons test | Summary | P Value |
| 38 vs. 94 | ns | 0.0659 |
| 38 vs. 76 | ** | 0.001 |
| 38 vs. hDD90-118 | * | 0.0121 |
| 38 vs. 116 | * | 0.0158 |
| 38 vs. 147 | ns | 0.225 |
| 94 vs. 76 | ns | 0.1884 |
| 94 vs. hDD90-118 | ns | 0.9066 |
| 94 vs. 116 | ns | 0.9519 |
| 94 vs. 147 | ** | 0.0012 |
| 76 vs. hDD90-118 | ns | 0.6554 |
| 76 vs. 116 | ns | 0.5638 |
| 76 vs. 147 | **** | <0.0001 |
| hDD90-118 vs. 116 | ns | >0.9999 |
| hDD90-118 vs. 147 | *** | 0.0003 |
| 116 vs. 147 | *** | 0.0003 |

FIG. 15B

| Šidák's multiple | | |
|---|---|---|
| comparisons test | | Adjusted |
| Pre - Post | Summary | P Value |
| hDD90-118 | ** | 0.0035 |
| 38 | * | 0.0206 |
| 76 | ns | 0.388 |
| 94 | ns | >0.9999 |
| 116 | ns | 0.3117 |
| 147 | **** | <0.0001 |

| | | |
|---|---|---|
| Tukey's multiple | | Adjusted |
| comparisons test | Summary | P Value |

FIG. 16

| | | |
|---|---|---|
| 10:1 vs. 25:1 | **** | <0.0001 |
| 10:1 vs. 50:1 | **** | <0.0001 |
| 10:1 vs. 75:1 | **** | <0.0001 |
| 10:1 vs. 100:1 | **** | <0.0001 |
| 25:1 vs. 50:1 | ns | >0.9999 |
| 25:1 vs. 75:1 | ns | 0.1448 |
| 25:1 vs. 100:1 | ns | 0.147 |
| 50:1 vs. 75:1 | ns | 0.1313 |
| 50:1 vs. 100:1 | ns | 0.1333 |
| 75:1 vs. 100:1 | ns | >0.9999 |

FIG. 18B

| | | |
|---|---|---|
| Lungs vs. Kidney | **** | <0.0001 |
| Lungs vs. Heart | **** | <0.0001 |
| Lungs vs. Spleen | **** | <0.0001 |
| Lungs vs. Lymph Node | **** | <0.0001 |
| Lungs vs. Liver | **** | <0.0001 |
| Kidney vs. Heart | ns | 0.7768 |
| Kidney vs. Spleen | ns | 0.1086 |
| Kidney vs. Lymph Node | ns | 0.9356 |
| Kidney vs. Liver | ns | 0.1107 |
| Heart vs. Spleen | ns | 0.6241 |
| Heart vs. Lymph Node | ns | 0.9985 |
| Heart vs. Liver | ns | 0.631 |
| Spleen vs. Lymph Node | ns | 0.4084 |
| Spleen vs. Liver | ns | >0.9999 |
| Lymph Node vs. Liver | ns | 0.4144 |

TABLE 5
NHP serum cytokine concentrations (pg/mL).

| | A2L084 | | A10R078 | | |
|---|---|---|---|---|---|
| Analyte | Predose | 4 hours | Predose | 24 hours | Units |
| G CSF | 0 | 12.4 | 0 | 0 | pg/mL |
| GMCSF | 0 | 0 | 0 | 0 | pg/mL |
| IFNg | 0 | 0 | 0 | 0 | pg/mL |
| IL-1b | 0 | 0 | 0 | 0 | pg/mL |
| IL-1Ra | 7.97 | 13.18 | 24.35 | 18 | pg/mL |
| IL-2 | 9.92 | 7.21 | 11.83 | 8.25 | pg/mL |
| IL-4 | 0 | 0 | 0 | 0 | pg/mL |
| IL-5 | 0 | 0 | 0 | 0 | pg/mL |
| IL-6 | 0 | 0 | 0 | 0 | pg/mL |
| IL-8 | 2221.5 | 3509 | 533.6 | 768.9 | pg/mL |
| IL-10 | 0 | 0 | 0 | 0 | pg/mL |
| IL-12/23 (p40) | 0 | 0 | 0 | 0 | pg/mL |
| IL-13 | 0 | 0 | 0 | 0 | pg/mL |
| IL-15 | 2.3 | 3.37 | 2.29 | 0 | pg/mL |
| IL-17A | 0 | 0 | 0 | 0 | pg/mL |
| IL-18 | 0 | 0 | 0 | 0 | pg/mL |
| MCP-1 | 171 | 229.67 | 149.9 | 112 | pg/mL |
| MIP-1b | 10.4 | 11.01 | 23.3 | 12.67 | pg/mL |
| MIP-1a | 0 | 0 | 0 | 0 | pg/mL |
| sCD40L | 19032 | 3049.2 | 4914.5 | 1310.6 | pg/mL |
| TGFa | 0 | 11.1 | 0 | 0 | pg/mL |
| TNFa | 0 | 0 | 0 | 0 | pg/mL |
| VEGF | 64.76 | 50.4 | 93.7 | 71.63 | pg/mL |

TABLE 6
NHP blood chemistry panel.

| | | | A10R078 | | | |
|---|---|---|---|---|---|---|
| | A2L084 | | | 24 | | |
| Metric | Predose | 4 hours | Predose | hours | Normal | Units |
| Glucose | 63 | 61 | 55 | 68 | 51-83 | mg/dL |
| BUN | 15 | 17 | 17 | 17 | 18-28 | mg/dL |
| Creatinine | 0.7 | 0.7 | 1 | 0.9 | 0.39-1.1 | mg/dL |
| Sodium | 150 | 151 | 150 | 150 | 138-152 | mmol/L |
| Potassium | 4.1 | 4.1 | 3.7 | 3.6 | 3.1-4.5 | mmol/L |
| Chloride | 112 | 113 | 110 | 110 | 99-115 | mmol/L |
| Anion Gap | 16 | 18.6 | 19.7 | 17.9 | 18.7-34.4 | mmol/L |
| CO2 | 26.1 | 23.5 | 24 | 25.7 | 10.8-24.8 | mmol/L |
| Phosphorus | 3.3 | 5 | 5.5 | 4.9 | 2.7-7.8 | mg/dL |
| Calcium | 9.2 | 9.6 | 9.3 | 9.2 | 8.2-10.2 | mg/dL |
| Tot. Protein | 7.6 | 7.7 | 7.8 | 7.8 | 5.4-7.8 | g/dL |
| Albumin | 3.2 | 3.3 | 3.7 | 3.7 | 3.0-4.2 | g/dL |
| Globulin | 4.4 | 4.4 | 4.1 | 4.1 | 1.0-4.8 | g/dL |
| Alb/Glob ratio | 0.7 | 0.8 | 0.9 | 0.9 | 0.9-1.8 | |
| Total Bilirubin | 0.2 | 0.2 | 0.2 | 0.2 | 0.0-0.5 | mg/dL |
| LDH | 202 | 158 | 428 | 429 | 153-764 | U/L |
| GGT | 311 | 311 | 58 | 56 | 28-106 | U/L |
| ALK Phos | 235 | 243 | 159 | 153 | 52-581 | U/L |
| ALT | 112 | 122 | 40 | 74 | 22-71 | U/L |
| AST | 26 | 34 | 24 | 79 | 14-48 | U/L |

TABLE 7
NHP hematology panel.

| | A2L084 | | A10R078 | | | |
|---|---|---|---|---|---|---|
| Metric | Predose | 4 hours | Predose | 24 hours | Normal | Units |
| WBC | 9 | 6.1 | 11.8 | 9.8 | 5.8-13.8 | ×10³/μL |
| RBC | 6.1 | 6.18 | 5.16 | 5.12 | 4.72-5.92 | ×10⁶/μL |
| Hemoglobin | 12.7 | 12.8 | 12.8 | 12.8 | 11.0-14.0 | g/dL |
| Hematocrit | 39.4 | 39.6 | 39.5 | 39 | 35.9-41.9 | % |
| MCV | 64.6 | 64.1 | 76.5 | 76.3 | 69.9-75.9 | fl |
| MCH | 20.8 | 20.8 | 24.8 | 24.9 | 21.9-24.9 | pg |
| MCHC | 32.2 | 32.4 | 32.5 | 32.7 | 31.2-34.4 | g/dL |
| RDW | 15 | 14.8 | 14.8 | 14.6 | | % |
| Platelet Ct. | 379 | 385 | 409 | 416 | 311-511 | ×10³/μL |
| MPV | 9.6 | 9.1 | 9.3 | 8.6 | | fl |
| Neut | 68.5 | 56.9 | 74.8 | 63.7 | 37.3-77.3 | % |
| Lymph | 20.5 | 30.5 | 20.4 | 28.4 | 18.8-54.8 | % |
| Mono | 5.9 | 7.2 | 3.4 | 6.8 | 0.0-6.9 | % |
| Eos | 4.7 | 5.3 | 1.1 | 1 | 0.0-3.0 | % |
| Baso | 0.4 | 0.1 | 0.3 | 0.1 | 0.0-1.0 | % |
| Neut | 6.2 | 3.5 | 8.8 | 6.2 | | ×10³/μL |
| Lymph | 1.8 | 1.8 | 2.4 | 2.8 | | ×10³/μL |
| Mono | 0.5 | 0.4 | 0.4 | 0.7 | | ×10³/μL |
| Eos | 0.4 | 0.3 | 0.1 | 0.1 | | ×10³/μL |
| Baso | 0 | 0 | 0 | 0 | | ×10³/μL |

### REFERENCES

1. Pardi, N. et al. Expression kinetics of nucleoside-modified mRNA delivered in lipid nanoparticles to mice by various routes. J. Control. Release Off. J. Control. Release Soc. 217, 345-351 (2015).
2. Tiwari, P. M. et al. Engineered mRNA-expressed antibodies prevent respiratory syncytial virus infection. Nat. Commun. 9, 3999 (2018).
3. Qiu, Y. et al. Effective mRNA pulmonary delivery by dry powder formulation of PEGylated synthetic KL4 peptide. J. Controlled Release 314, 102-115 (2019).
4. Lokugamage, M. P. et al. Optimization of lipid nanoparticles for the delivery of nebulized therapeutic mRNA to the lungs. Nat. Biomed. Eng. 5, 1059-1068 (2021).
5. Chollet, P., Favrot, M. C., Hurbin, A. & Coll, J.-L. Side-effects of a systemic injection of linear polyethylenimine-DNA complexes. J. Gene Med. 4, 84-91 (2002).
6. Wen, Y. et al. A Biodegradable Low Molecular Weight Polyethylenimine Derivative as Low Toxicity and Efficient Gene Vector. Bioconjug. Chem. 20, 322-332 (2009).
7. Translate Bio Announces Results from Second Interim Data Analysis from Ongoing Phase 1/2 Clinical Trial of MRT5005 in Patients with Cystic Fibrosis (CF). https://investors.translate.bio/news-releases/news-release-details/translate-bio-announces-results-second-interim-data-analysis/(2021).
8. Kaczmarek, J. C. et al. Polymer-Lipid Nanoparticles for Systemic Delivery of mRNA to the Lungs. Angew. Chem. Int. Ed Engl. 55, 13808-13812 (2016).
9. Patel, A. K. et al. Inhaled nanoformulated mRNA polyplexes for protein production in lung epithelium. Adv. Mater. 31, 1805116 (2019).
10. Blanchard, E. L. et al. Treatment of influenza and SARS-COV-2 infections via mRNA-encoded Cas13a in rodents. Nat. Biotechnol. 39, 717-726 (2021).
11. Martini, V. et al. Distribution of Droplets and Immune Responses After Aerosol and Intra-Nasal Delivery of Influenza Virus to the Respiratory Tract of Pigs. Front. Immunol. 11, 594470 (2020).
12. Samuel, J. & Smaldone, G. C. Maximizing Deep Lung Deposition in Healthy and Fibrotic Subjects During Jet Nebulization. J. Aerosol Med. Pulm. Drug Deliv. 33, 108-115 (2020).
13. Suchý, M., Elmehriki, A. A. H. & Hudson, R. H. E. A Remarkably Simple Protocol for the N-Formylation of Amino Acid Esters and Primary Amines. Org. Lett. 13, 3952-3955 (2011).
14. Yang, D.-S. & Jeon, H.-B. Convenient N-Formylation of Amines in Dimethylformamide with Methyl Benzoate under Microwave Irradiation. Bull. Korean Chem. Soc. 31, 1424-1426 (2010).
15. Sonawane, R. B., Rasal, N. K., Bhange, D. S. & Jagtap, S. V. Copper-(II) Catalyzed N-Formylation and N-Acylation of Aromatic, Aliphatic, and Heterocyclic Amines and a Preventive Study in the C—N Cross Coupling of Amines with Aryl Halides. ChemCatChem 10, 3907-3913 (2018).
16. Tan, Z., Li, Z., Ma, Y., Qin, J. & Yu, C. Potassium tert-Butoxide Prompted Highly Efficient Transamidation and Its Coordination Radical Mechanism. Eur. J. Org. Chem. 2019, 4538-4545 (2019).
17. Sakurai, M., Kawakami, R. & Kihara, N. TBSOTf-promoted versatile N-formylation using DMF at room temperature. Tetrahedron Lett. 60, 1291-1294 (2019).
18. Wang, Y. et al. Transformylating amine with DMF to formamide over CeO2 catalyst. Chem. Commun. 50, 2438-2441 (2014).
19. Lindsay, K. E. et al. Aerosol delivery of synthetic mRNA to vaginal mucosa leads to durable expression of broadly neutralizing antibodies against HIV. Mol. Ther. J. Am. Soc. Gene Ther. 28, 805-819 (2020).
20. Li, L. et al. The dose of HBV genome contained plasmid has a great impact on HBV persistence in hydrodynamic injection mouse model. Virol. J. 14, 205 (2017).
21. Tankersley, C. G., Fitzgerald, R. S. & Kleeberger, S. R. Differential control of ventilation among inbred strains of mice. Am. J. Physiol.-Regul. Integr. Comp. Physiol. 267, R1371-R1377 (1994).
22. Kida, K., Fujino, Y. & Thurlbeck, W. M. A Comparison of Lung Structure in Male DBA and C57 Black Mice and Their F1 Offspring. Am. Rev. Respir. Dis. 139, 1238-1243 (1989).
23. Hatit, M. Z. C. et al. Species-dependent in vivo mRNA delivery and cellular responses to nanoparticles. Nat. Nanotechnol. 17, 310-318 (2022).
24. Liu, Z. et al. Systematic comparison of 2A peptides for cloning multi-genes in a polycistronic vector. Sci. Rep. 7, 1-9 (2017).
25. Nelson, J. et al. Impact of mRNA chemistry and manufacturing process on innate immune activation. Sci. Adv. 6, caaz6893 (2020).
26. Altamirano-Lagos, M. J. et al. Current Animal Models for Understanding the Pathology Caused by the Respiratory Syncytial Virus. Front. Microbiol. 10, 873 (2019).
27. Zost, S. J. et al. Potently neutralizing and protective human antibodies against SARS-CoV-2. Nature 584, 443-449 (2020).
28. An, D. et al. Protection of K18-hACE2 mice and ferrets against SARS-COV-2 challenge by a single-dose mucosal immunization with a parainfluenza virus 5-based COVID-19 vaccine. Sci. Adv. 7, eabi5246 (2021).
29. Haque, A. K. M. A. et al. Chemically modified hCFTR mRNAs recuperate lung function in a mouse model of cystic fibrosis. Sci. Rep. 8, 16776 (2018).
30. Tsuji, Y. et al. Human sperm carbohydrate antigens defined by an antisperm human monoclonal antibody derived from an infertile woman bearing antisperm antibodies in her serum. J. Exp. Med. 168, 343-356 (1988).
31. Komori, S., Yamasaki, N., Shigeta, M., Isojima, S. & Watanabe, T. Production of heavy-chain class-switch variants of human monoclonal antibody by recombinant DNA technology. Clin. Exp. Immunol. 71, 508-516 (1988).
32. Rajamaki, K. et al. Cholesterol crystals activate the NLRP3 inflammasome in human macrophages: a novel link between cholesterol metabolism and inflammation. PLOS ONE 5, c11765 (2010).
33. Humphrey, W., Dalke, A. & Schulten, K. VMD: Visual molecular dynamics. J. Mol. Graph. 14, 33-38 (1996). 34. Abula, A. et al. Molecular mechanism of RNase R substrate sensitivity for RNA ribose methylation. Nucleic Acids Res. 49, 4738-4749 (2021).
35. Jorgensen, W. L., Chandrasekhar, J., Madura, J. D., Impey, R. W. & Klein, M. L. Comparison of simple potential functions for simulating liquid water. J. Chem. Phys. 79, 926-935 (1983).
36. Phillips, J. C. et al. Scalable molecular dynamics on CPU and GPU architectures with NAMD. J. Chem. Phys. 153, 044130 (2020).
37. Phillips, J. C. et al. Scalable Molecular Dynamics with NAMD. J. Comput. Chem. 26, 1781-1802 (2005).
38. Vanommeslaeghe, K. et al. CHARMM General Force Field (CGenFF): A force field for drug-like molecules compatible with the CHARMM all-atom additive biological force fields. J. Comput. Chem. 31, 671-690 (2010).
39. Yu, W., He, X., Vanommeslaeghe, K. & MacKerell, A. D. Extension of the CHARMM General Force Field to Sulfonyl-Containing Compounds and Its Utility in Biomolecular Simulations. J. Comput. Chem. 33, 2451-2468 (2012).
40. Denning, E. J., Priyakumar, U. D., Nilsson, L. & MacKerell, A. D. Impact of 2′-hydroxyl sampling on the conformational properties of RNA: Update of the CHARMM all-atom additive force field for RNA. J. Comput. Chem. 32, 1929-1943 (2011).
41. Feller, S. E., Zhang, Y., Pastor, R. W. & Brooks, B. R. Constant pressure molecular dynamics simulation: The Langevin piston method. J. Chem. Phys. 103, 4613-4621 (1995).
42. Balusek, C. et al. Accelerating membrane simulations with Hydrogen Mass Repartitioning. J. Chem. Theory Comput. 15, 4673-4686 (2019).
43. Darden, T., York, D. & Pedersen, L. Particle mesh Ewald: An N.log (N) method for Ewald sums in large systems. J. Chem. Phys. 98, 10089-10092 (1993).
44. Hunter, R. J. Zeta Potential in Colloid Science, Principles and Applications. https://www.elsevier.com/books/zeta-potential-in-colloid-science/hunter/978-0-12-361961-7 (1988).
45. Honary, S. & Zahir, F. Effect of Zeta Potential on the Properties of Nano-Drug Delivery Systems-A Review. Trop. J. Pharm. Res. 12, 255-264 (2013).
46. Schwegmann, H., Feitz, A. J. & Frimmel, F. H. Influence of the zeta potential on the sorption and toxicity of iron oxide nanoparticles on S. cerevisiae and E. coli. J. Colloid Interface Sci. 347, 43-48 (2010).
47. Patel, A. K. et al. Inhaled nanoformulated mRNA polyplexes for protein production in lung epithelium. Adv. Mater. 31, 1805116 (2019).

All references listed in this document are hereby incorporated by reference in their entirety as if fully set forth herein.

What is claimed is:

1. A method of preventing or treating or controlling a respiratory disease in a bovine comprising:
delivering to a lung or a trachea of the bovine a pharmaceutical composition comprising a pharmaceutically acceptable carrier and a polyplex,
wherein the polyplex comprises:
one or more, optionally further modified, mRNA encoded with a molecule selected from the group consisting of glycosylphosphatidylinositol (GPI) complex, antibody immunoglobulin G (IgG), antibody immunoglobulin A (IgA), antibody immunoglobulin M (IgM), antimicrobial peptide Bactenecin 5 (Bac5), antimicrobial peptide Bactenecin 7 (Bac7), bovine myeloid antimicrobial peptide (BMAP-28), lipoxin inducing enzymes, resolvin inducing enzymes, cytokines, CRISPR associated protein 13 (Cas13) enzymes, nanoluciferase (NLuc) proteins, and gene activator, catalytically dead CRISPR-associated protein 9 fused to an activator selected from the group consisting of VP64-p65-Rta transactivation domain (dCas9-VPR), VP64, and VP64-p65-HSF1 on the N terminus and SS18 on the C-terminus,
a polymer prepared by
a. polymerizing components i-ii and optionally iii:
i. a diacrylate monomer;
ii. one or more linker monomers including one or more functional groups independently selected from the group consisting of amino groups, alcohol groups and thiol groups; and
iii. one or more branching monomers including one or more functional groups independently selected from the group consisting of amino groups, alcohol groups and thiol groups; and
b. end-capping the product of step (a) with one or more end capping monomers including one or more functional groups independently selected from the group consisting of amino groups and alcohol groups.

2. The method of claim 1, wherein the pharmaceutical composition further comprises an RNA, an antibody, a cytokine, or combinations thereof;
wherein the RNA is selected from the group consisting of messenger RNA (mRNA), cargo RNA (cRNA), guide RNA (gRNA), transfer RNA (tRNA), ribosomal RNA (rRNA), GPI anchored RNA, and CRISPR RNA (crRNA), optionally, the RNA is selected from the group consisting of mRNA, CRNA, gRNA, anchored RNA, and crRNA;
wherein the antibody is selected from the group consisting of IgG, IgA, and IgM; and
wherein the cytokine is optionally selected from the group consisting of interferon and lambda.

3. The method of claim 1, wherein the pharmaceutical composition comprises two or more mRNA wherein each mRNA is individually encoded with a molecule selected from the group consisting of glycosylphosphatidylinositol (GPI) complex, antibody immunoglobulin G (IgG), antibody immunoglobulin A (IgA), antibody immunoglobulin M (IgM), antimicrobial peptide Bactenecin 5 (Bac5), antimicrobial peptide Bactenecin 7 (Bac7), bovine myeloid antimicrobial peptide (BMAP-28), lipoxin inducing enzymes, resolvin inducing enzymes, cytokines, CRISPR associated protein 13 (Cas13) enzymes, nanoluciferase (NLuc) proteins, and gene activator, catalytically dead CRISPR-associated protein 9 fused to an activator selected from the group consisting of VP64-p65-Rta transactivation domain (dCas9-VPR), VP64, and VP64-p65-HSF1 on the N terminus and SS18 on the C-terminus.

4. The method of claim 1, wherein the pharmaceutical composition comprises three or more mRNA wherein each mRNA is individually encoded with a molecule selected from the group consisting of glycosylphosphatidylinositol (GPI) complex, antibody immunoglobulin G (IgG), antibody immunoglobulin A (IgA), antibody immunoglobulin M (IgM), antimicrobial peptide Bactenecin 5 (Bac5), antimicrobial peptide Bactenecin 7 (Bac7), bovine myeloid antimicrobial peptide (BMAP-28), lipoxin inducing enzymes, resolvin inducing enzymes, cytokines, CRISPR associated protein 13 (Cas13) enzymes, nanoluciferase (NLuc) proteins, and gene activator, catalytically dead CRISPR-associated protein 9 fused to an activator selected from the group consisting of VP64-p65-Rta transactivation domain (dCas9-VPR), VP64, and VP64-p65-HSF1 on the N terminus and SS18 on the C-terminus.

5. The method of claim 1, wherein the pharmaceutical composition further comprises crRNA.

6. The method of claim 1, wherein the pharmaceutical composition is delivered via a nebulizer, vibrating mesh nebulizer, jet nebulizer, intratracheal instillation, intranasal instillation, and aerosol or instillation to oral, nasal, and pharyngeal membranes.

7. The method of claim 6, wherein the pharmaceutical composition is delivered via the nebulizer which produces nebulized droplets having an average diameter of from about 1 μm-10 μm, as measured by dynamic light scattering.

8. The method of claim 1, wherein the bovine is selected from the group consisting of calf, heifer, adult cow, steer, and bull.

9. The method of claim 1, wherein the respiratory disease is selected from the group consisting of pneumonia, pleuritis, pleuropneumonia, bronchitis, respiratory syncytial virus (RSV) infection, influenza, coronavirus, infectious bovine herpes virus, bovine viral diarrhea virus, influenza, bovine rhinovirus, bovine rhinitis virus, Mannheimia haemolytica, Pasteurella multocida, Histophilus somni, and Mycoplasma species.

10. The method of claim 1, wherein the method comprises treating or controlling the respiratory disease wherein the bovine is already infected with the respiratory disease.

11. The method of claim 1, wherein the polymer comprises the one or more branching monomers.

12. The method of claim 11, wherein the polymer has a molar ratio of the diacrylate monomer to the one or more linker monomers to the one or more branching monomers to the one or more end capping monomers is from about 1.0:0.25:0:1.0 to 1.0:1.0:1.0:2.0.

13. The method of claim 1, wherein the one or more diacrylates of the polymer is selected from the group consisting of:

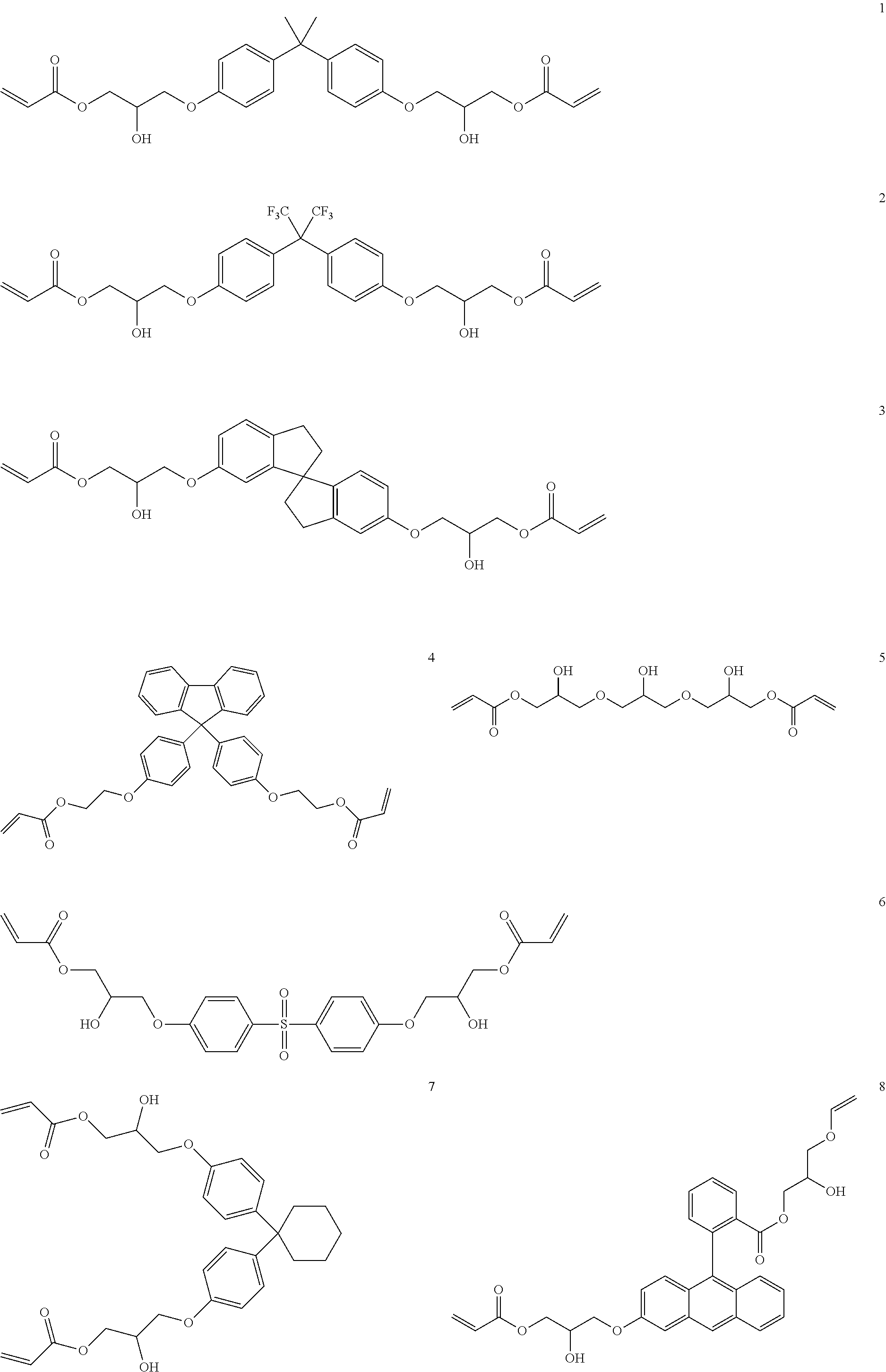

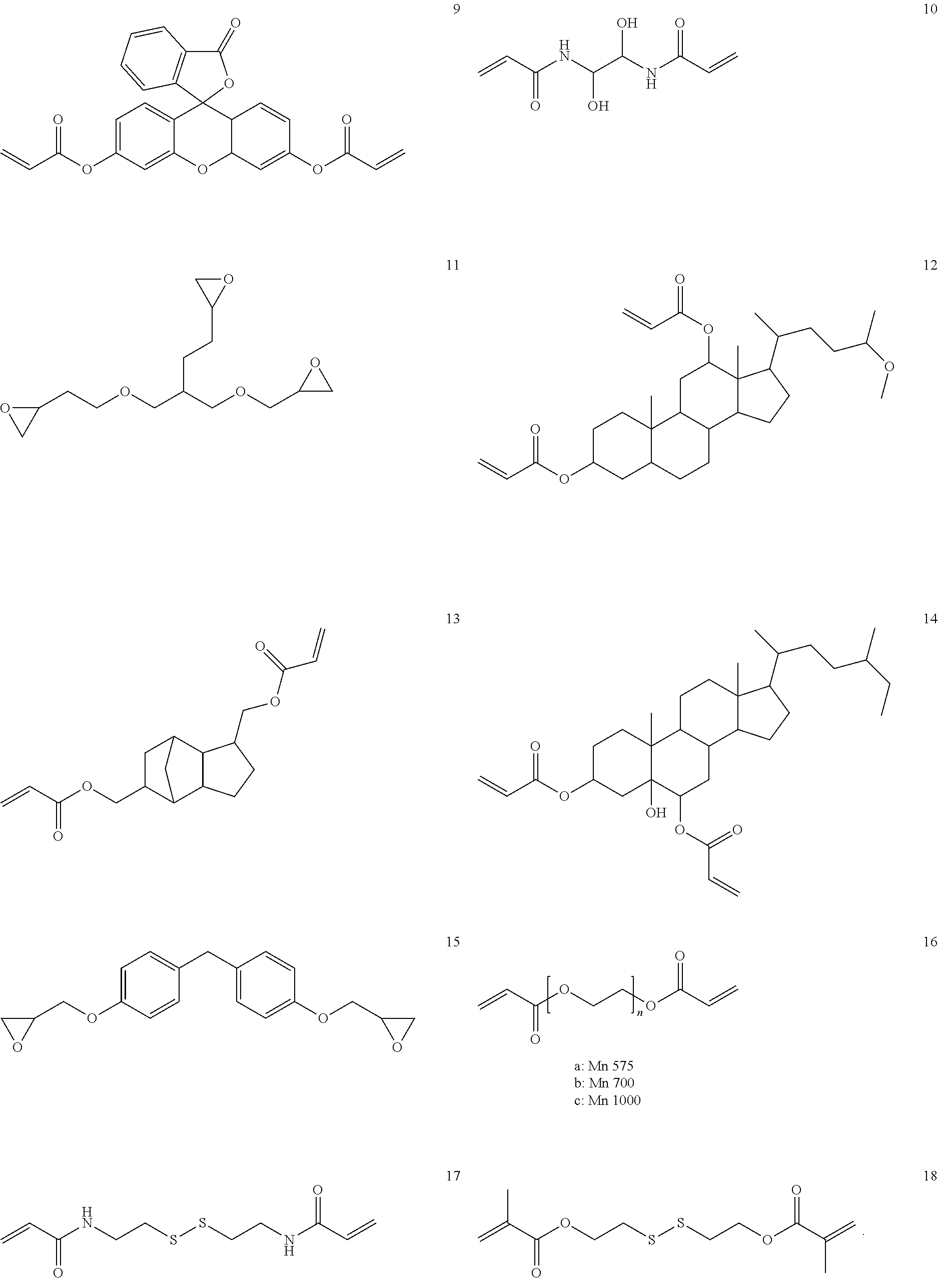

14. The method of claim 1, wherein the one or more linker monomers is selected from the group consisting of:

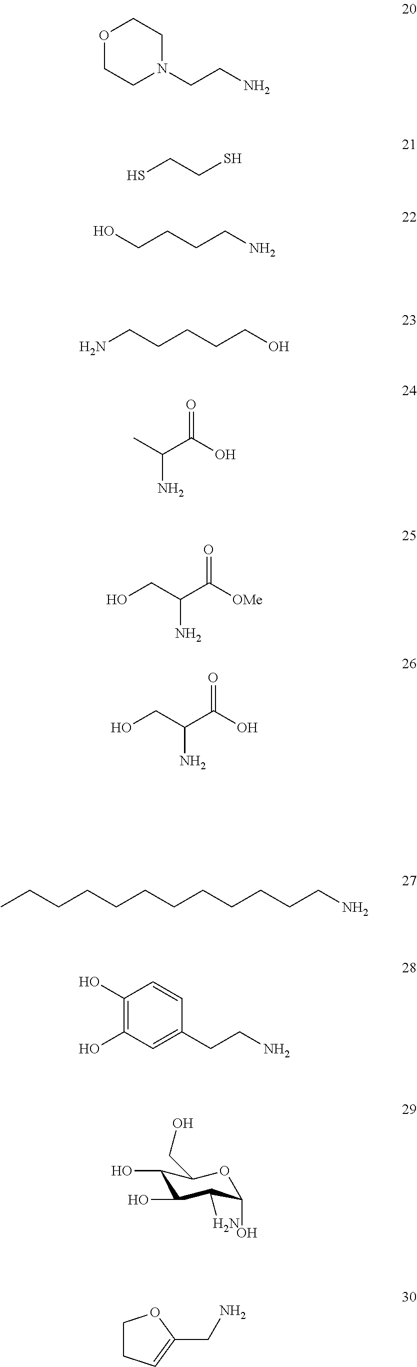

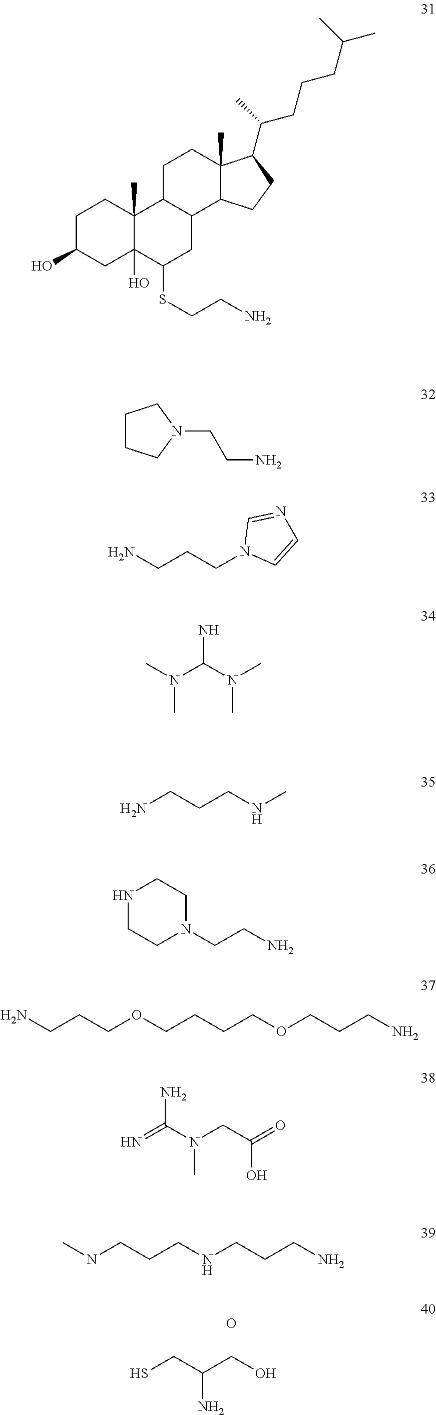

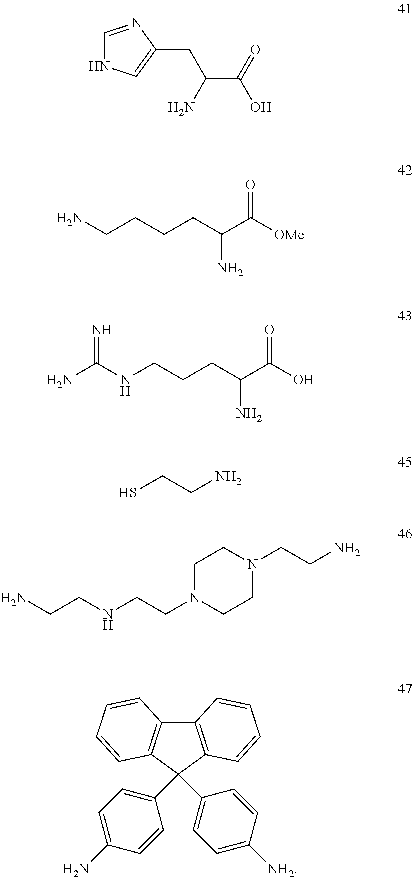

15. The method of claim 1, wherein the one or more branching monomers is selected from the group consisting of:

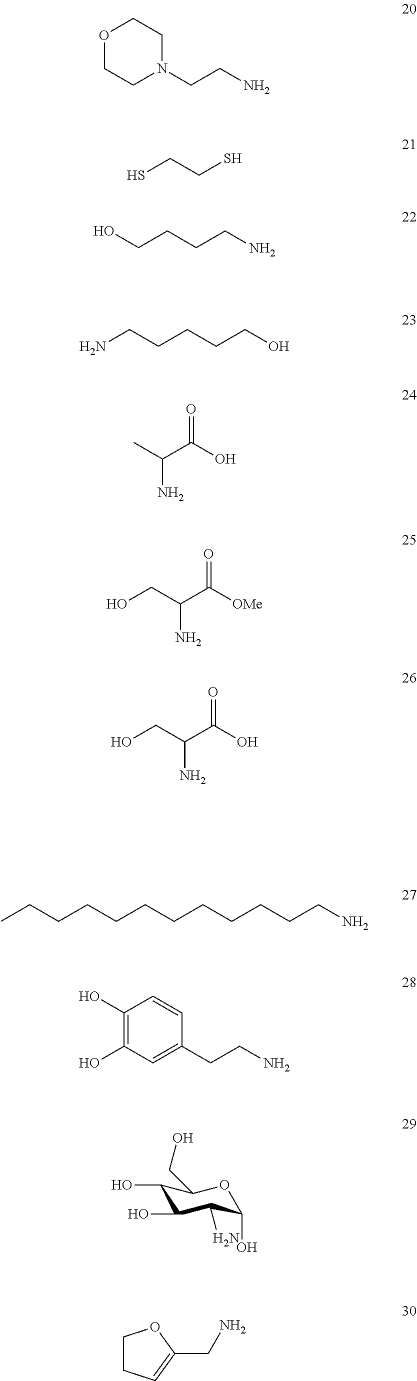

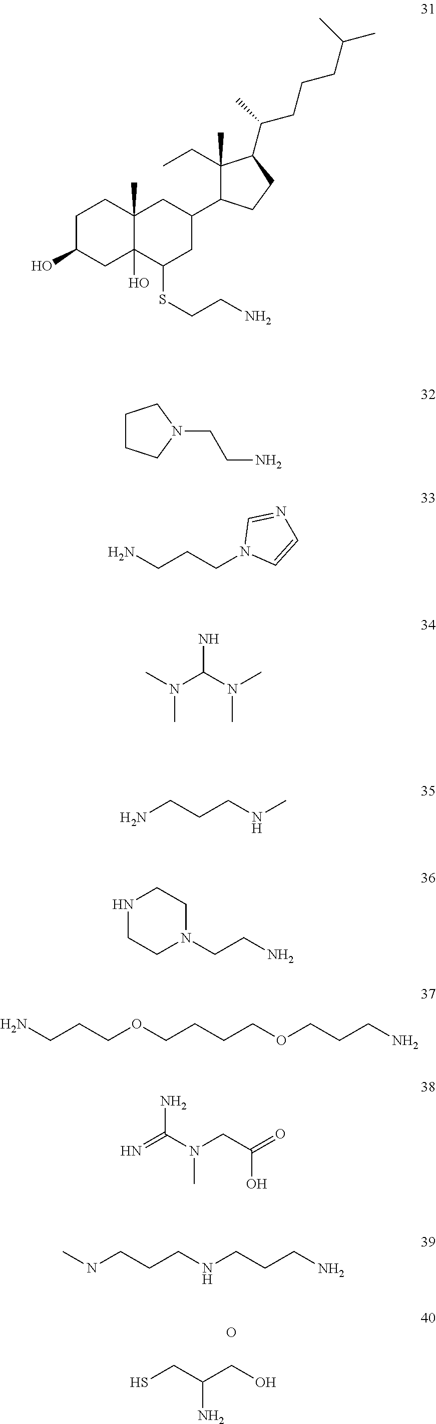

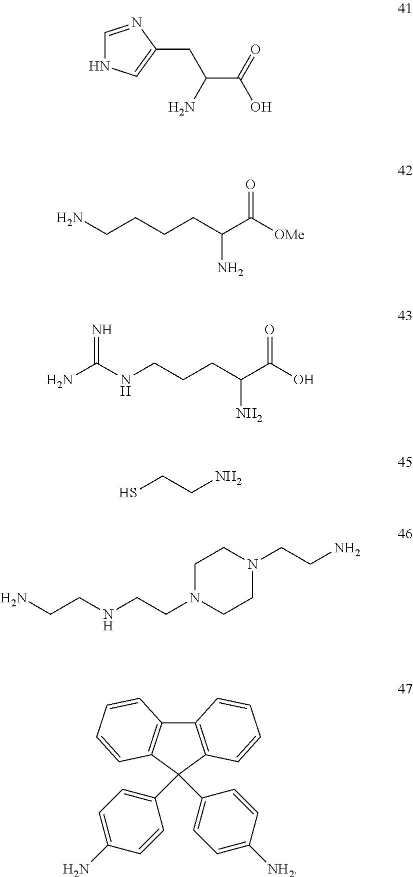

16. The method of claim 1, wherein the one or more end capping monomers is selected from the group consisting of:

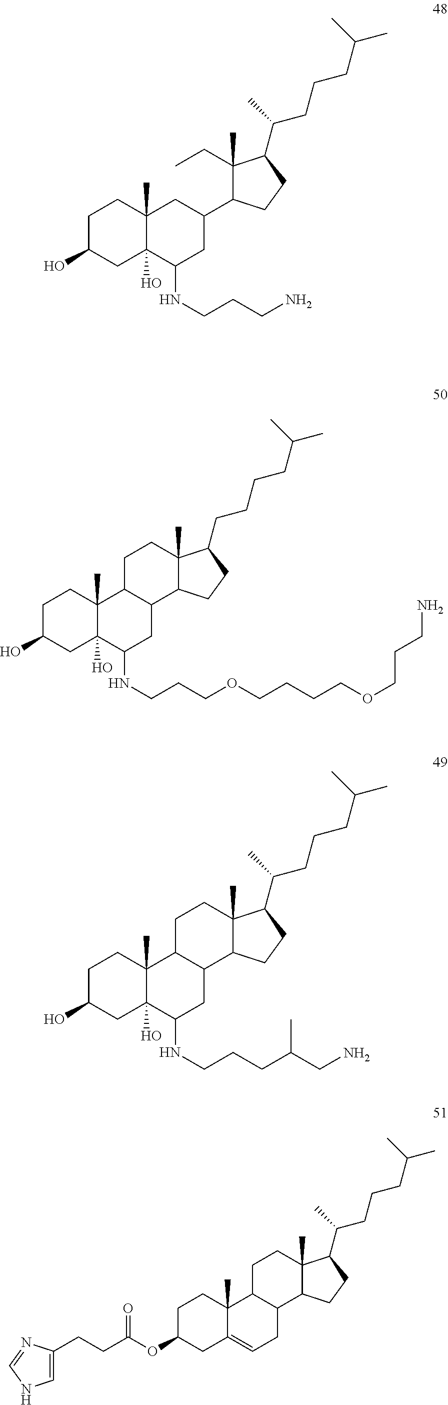

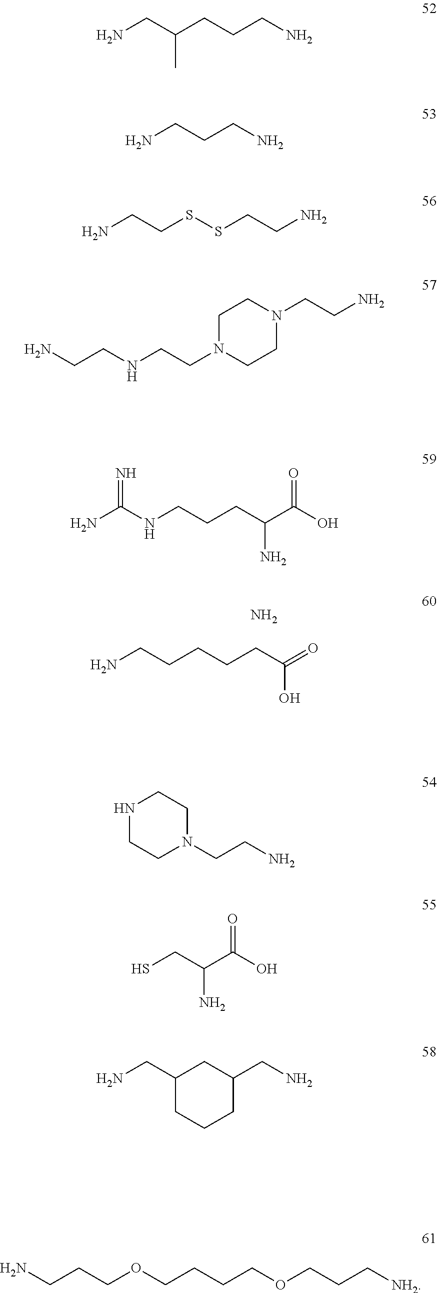

17. The method of claim 1, wherein the polymer is selected from the group consisting of:

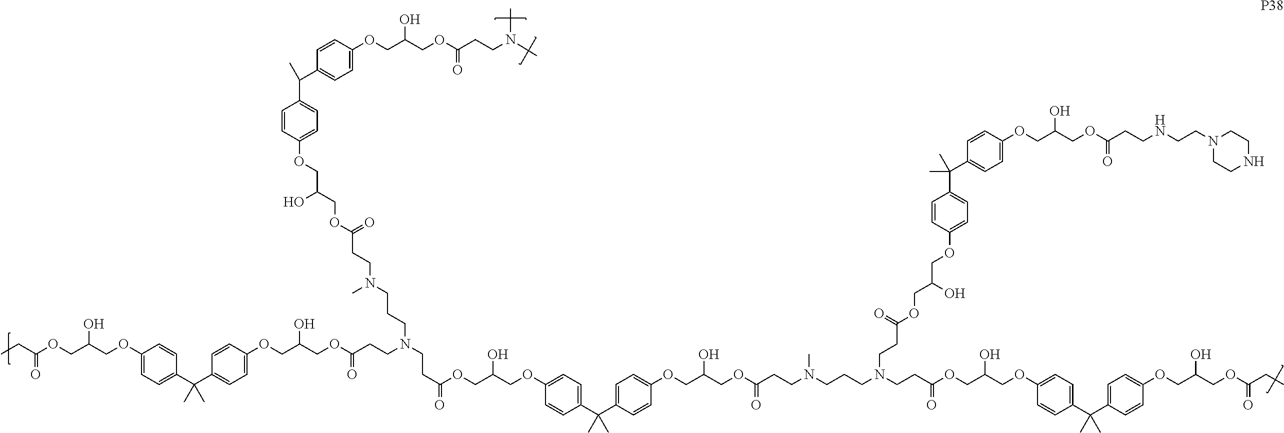

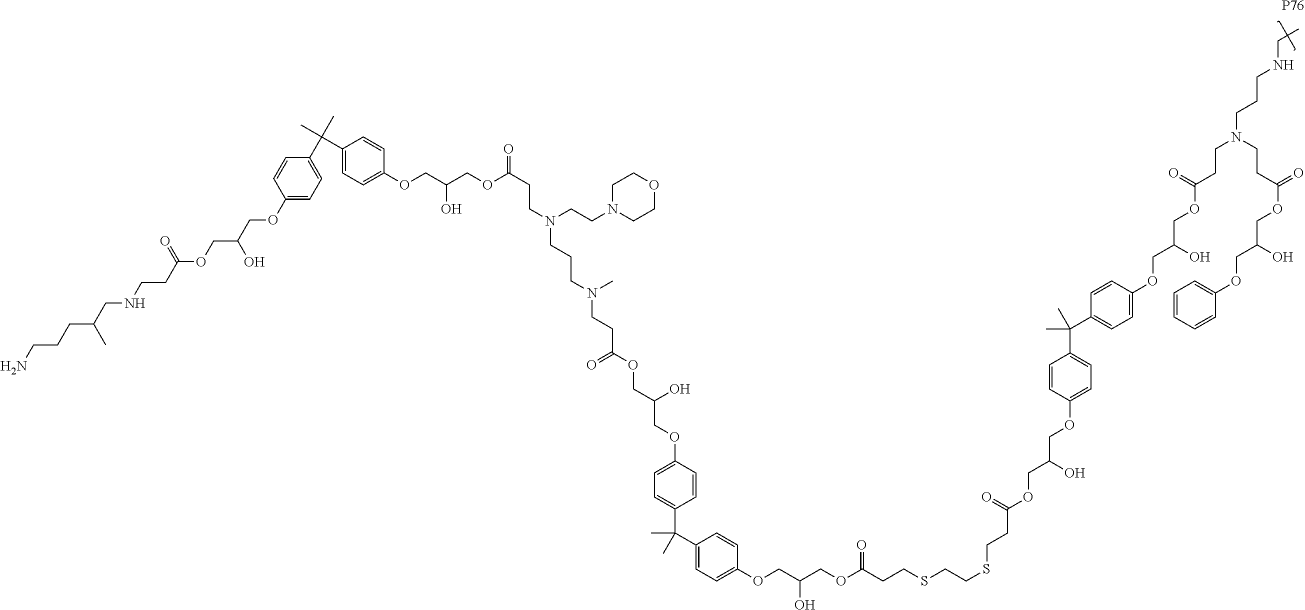 [SMILES: CCNCCCN(CCC(=O)OCC(O)COc1ccccc1)CCC(=O)OCC(O)COc1ccc(C(C)(C)c2ccc(OCC(O)COC(=O)CCSCCSCCC(=O)OCC(O)COc3ccc(C(C)(C)c4ccc(OCC(O)COC(=O)CCN(C)CCCN(CCC(=O)OCC(O)COc5ccc(C(C)(C)c6ccc(OCC(O)COC(=O)CCNCC(C)CCCN)cc6)cc5)CCN5CCOCC5)cc4)cc3)cc2)cc1 ; "P76"]

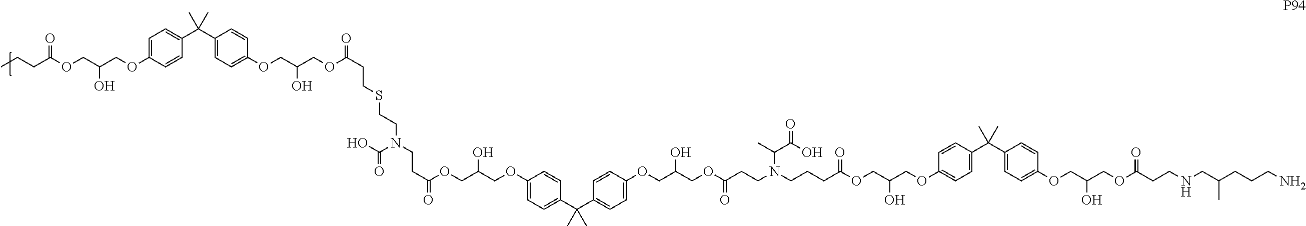 [SMILES: CCCC(=O)OCC(O)COc1ccc(C(C)(C)c2ccc(OCC(O)COC(=O)CCSCCN(CCC(=O)OCC(O)COc3ccc(C(C)(C)c4ccc(OCC(O)COC(=O)CCN(CCCC(=O)OCC(O)COc5ccc(C(C)(C)c6ccc(OCC(O)COC(=O)CCNCC(C)CCCN)cc6)cc5)C(C)C(=O)O)cc4)cc3)C(=O)O)cc2)cc1 ; "P94"]

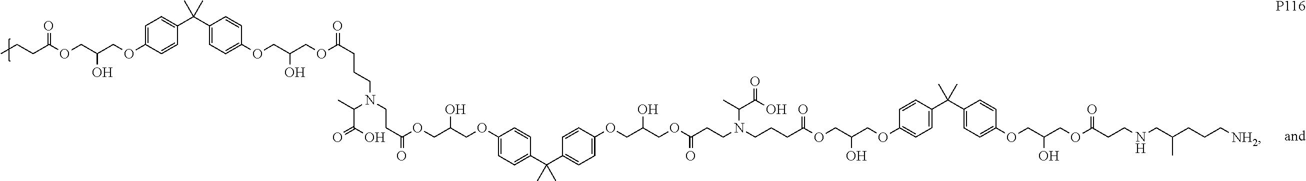 [SMILES: CCCC(=O)OCC(O)COc1ccc(C(C)(C)c2ccc(OCC(O)COC(=O)CCCN(CCC(=O)OCC(O)COc3ccc(C(C)(C)c4ccc(OCC(O)COC(=O)CCN(CCCC(=O)OCC(O)COc5ccc(C(C)(C)c6ccc(OCC(O)COC(=O)CCNCC(C)CCCN)cc6)cc5)C(C)C(=O)O)cc4)cc3)C(C)C(=O)O)cc2)cc1 ; "P116"]

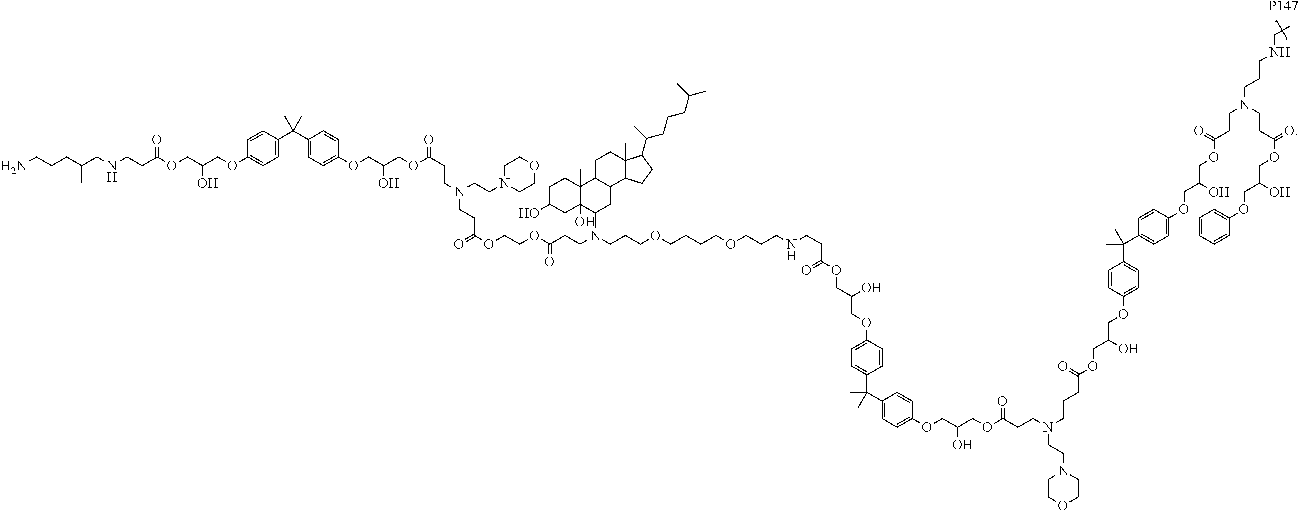 [SMILES: CCNCCCN(CCC(=O)OCC(O)COc1ccccc1)CCC(=O)OCC(O)COc1ccc(C(C)(C)c2ccc(OCC(O)COC(=O)CCCN(CCC(=O)OCC(O)COc3ccc(C(C)(C)c4ccc(OCC(O)COC(=O)CCNCCCOCCCCOCCCN(CCC(=O)OCCOC(=O)CCN(CCC(=O)OCC(O)COc5ccc(C(C)(C)c6ccc(OCC(O)COC(=O)CCNCC(C)CCCN)cc6)cc5)CCN5CCOCC5)C5CC6C7CCC(C(C)CCCC(C)C)C7(C)CCC6C6(C)CCC(O)CC56O)cc4)cc3)CCN3CCOCC3)cc2)cc1 ; "P147"]

18. The method of claim 1, wherein the polymer is selected from the group consisting of a poly-beta-amino-thio-ester-polymer and a poly-beta-amino-ester polymer.

19. The method of claim 1, wherein the one or more mRNA is encoded with a molecule selected from the group consisting of glycosylphosphatidylinositol (GPI) complex, antibody immunoglobulin G (IgG), antibody immunoglobulin A (IgA), antibody immunoglobulin M (IgM), antimicrobial peptide Bactenecin 5 (Bac5), antimicrobial peptide Bactenecin 7 (Bac7), bovine myeloid antimicrobial peptide (BMAP-28), lipoxin inducing enzymes, resolvin inducing enzymes, cytokines, CRISPR associated protein 13 (Cas13) enzymes, nanoluciferase (NLuc) proteins, and gene activator, catalytically dead CRISPR-associated protein 9 fused to an activator selected from the group consisting of VP64-p65-Rta transactivation domain (dCas9-VPR), VP64, and VP64-p65-HSF1 on the N terminus and SS18 on the C-terminus.

20. The method of claim 1, wherein the one or more mRNA comprises the mRNA-expressed clustered regularly interspaced short palindromic repeat associated protein 13 (Cas13a).
